(19) United States
(12) Patent Application Publication
(10) Pub. No.: US 2026/0184726 A1
(43) Pub. Date: Jul. 2, 2026

(54) HETEROCYCLIC COMPOUNDS AS KINASE INHIBITORS, COMPOSITIONS, AND METHODS OF USE THEREOF

(71) Applicant: JS INNOMED HOLDINGS LTD., Grand Cayman (KY)

(72) Inventors: Qun LI, Sunnyvale, CA (US); Jintao ZHANG, Naperville, IL (US); Shanzhong JIAN, Shanghai (CN); Ao LI, Shanghai (CN); Xia YUAN, Shanghai (CN); Wen XU, Shanghai (CN)

(21) Appl. No.: 18/865,314

(22) PCT Filed: May 13, 2022
(86) PCT No.: PCT/CN2022/092713

§ 371 Date: Nov. 12, 2024

(51) Int. Cl. C07D 519/00; A61K 31/444; A61K 31/4748; A61K 31/497; A61K 31/4995; A61K 31/501; A61K 31/506; A61K 45/06; A61P 35/00; C07D 471/04

(52) U.S. Cl. CPC C07D 519/00; A61K 31/444; A61K 31/4748; A61K 31/497; A61K 31/4995; A61K 31/501; A61K 31/506; A61K 45/06; A61P 35/00; C07D 471/04

(57) ABSTRACT

Disclosed herein are compounds of Formula I, and/or stereoisomers, stable isotopes, or pharmaceutically acceptable salts or solvates thereof; and therapeutic uses of these compounds, which are inhibitors of rearranged during transfection (RET) and potentially useful in the treatment of RET-associated diseases, such as RET-associated cancers.

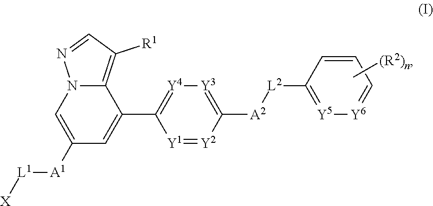

### TECHNICAL FIELD

Disclosed herein are novel heterocyclic compounds that can serve as rearranged during transfection (RET) kinase inhibitors. Further disclosed herein are pharmaceutical compositions, comprising at least one of such compounds, as well as methods of using at least one of such compounds in the treatment of diseases and disorders modulated by RET, such as cancers.

### TECHNICAL BACKGROUND

RET is a transmembrance glycoprotein receptor tyrosine kinase (RTK) that is encoded by RET oncogene (Borrello, M. G., et al., Expert Opin. Ther. Targets. 2013, vol. 17, pp. 403-419). Upon homodimerization mediated by the GFL-GFRα complex, RET is activated via trans-autophosphorylation on the tyrosine residues in the intracellular kinase domain. The phosphotyrosine residues of RET serve as docking sites for the SH2 domain of several signaling adaptors which activate several signal transduction cascades involved in cellular proliferation, including the RAS/MARK/ERK, PI3K/Akt/mTOR, and JAK/STAT pathweays. There are several major genetic aberrations leading to a dysregulated RET activity in many tumors. RET gene fusions and RET point mutations are RET mutations in many tumors, among others. RET gene fusions are found in a variety of cancers, including 1-2% non-small cell lung cancers (NSCLC), 20-30% of papillary thyroid cancers (PTCs), and less than 1% of other cancers such as pancreatic cancers, salivary gland cancers, spitz tumors, colorectal cancers, ovarian cancers and myeloproliferative cancers. So far at least 12 different fusion variants have been identified, with KIF5B-RET being the most common in NSCLCs, and CCDC6 and NCOA4 being most common in PTCs. RET point mutations occur mostly in sporadic medullary thyroid cancers (MTCs, 30-50%) and hereditary MTCs (100%), with RET M918T, G810R, V804L and V804M and being the most common mutations. Moreover, overexpression of wild-type RET, through its physiological neurotrophic functions, may play a role in the pathogenesis of other tumor types, such as pancreatic cancer.

Therefore, RET is a potential therapeutic target in cancer and other diseases with aberrant RET activity (such as a gastrointestinal disorder such as irritable bowel syndrome). A number of multitargeted kinase inhibitors with RET activity, such as cabozantinib, vandetanib, lenvatinib and alectinib, have been already investigated in clinical trials in cancer patients (Drilon, A. et al. Nat. Rev. Clin. Oncol., 2018, vol. 15, pp. 151-167). Depite showing efficacy in certain tumor types, the clinical activity of such multitargeted agents has been limited due to short duration and severe side effects. Such inhibitors, due to their dose-limiting toxicological liabilities caused by the primary and more potent inhibition of non-RET kinases, such as VEGFR2, have not to date allowed unequivocal demonstration of value of RET per se as a clinically relevant therapeutic target. Therefore, there is a need for more potent and more RET selective inhibitor drugs with better drug-like properties like improved DMPK properties.

### SUMMARY OF THE DISCLOSURE

Disclosed herein are novel potent and selective RET kinase inhibitors and methods for their preparation and uses thereof. The compounds disclosed herein can have strong cancer inhibitory effects and can effectively inhibit RET-associated cancers.

Disclosed herein are compounds of Formula I:

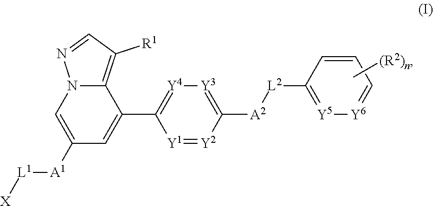

and/or stereoisomers, stable isotopes, or pharmaceutically acceptable salts or solvates thereof, wherein A¹, A², L¹, L², R¹, R², X, Y¹, Y², Y³, Y⁴, Y⁵, Y⁶, and n are defined below.
A¹ is a cyclic group selected from phenyl and 5- to 6-membered heteroaryl, wherein the 5- to 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl;
A² is a group selected from:

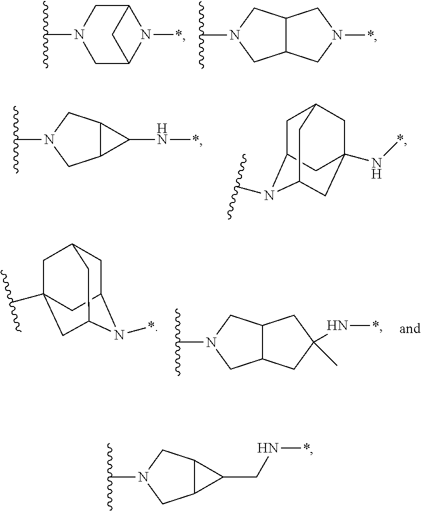

wherein the bond marked with an asterisk (*) represents the bond to L² of Formula I;
L¹ is a group selected from:

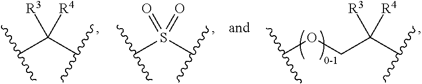

wherein R³ and R⁴ are independently selected from H and C1-C6 alkyl, wherein the C1-C6 alkyl is optionally substituted with 1-3 groups independently selected from CN, halo, OH, and C1-C6 alkoxy; or wherein R³ and R⁴ are taken together to form a group selected from oxo, 3- to 6-membered cycloalkyl, and 5- to 6-membered heterocycles containing 1-2 heteroatoms independently selected from N, O, and S as ring members;
L² is a group selected from CO, SO₁₋₂, C1-C6 alkylenyl, and C1-C6 haloalkylenyl:
R¹ is selected from —CN, ethynyl, halo, —CF₃, —CH₃, —CH₂CH₃, cyclopropyl, —CH₂CN, and —CH(CN)CH₃;
each R² is independently selected from halo, —OR⁵, —N(R⁵)₂, —CN, C1-C6 alkyl, C1-C6 haloalkyl, C3-C6 cycloalkyl, —OC(O)R⁵, —CO₂R⁵, —C(O)N(R⁵)₂, —C(═NR⁶)N(R⁵)₂, —C(O)R⁵, —S(O)₀₋₂R⁷, —S(O)(═NR⁶) R⁷, —S(O)₁₋₂N(R⁵)₂, —N(R⁵)C(O)R⁷, —N(R⁵)C(═NR⁶) R⁷, —N(R⁵)S(O)₁₋₂R⁷, —N(R⁵)C(O)N(R⁵)₂, —N(R⁵)C(═NR⁶)N(R⁵)₂, —N(R⁵)S(O)₁₋₂N(R⁵)₂, and —N(R⁵)CO₂R⁷, wherein:
each R⁵ is independently selected from H, C1-C6 alkyl, C3-C6 cycloalkyl, C1-C6 haloalkyl, aryl, saturated and unsaturated 4-7 membered heterocyclyl containing 1-2 heteroatoms selected from N, O, and S as ring members, and heteroaryl containing 1-4 heteroatoms selected from N, O, and S as ring members;
each R⁶ is independently selected from H, —CN, —OH, C1-C4 alkyl, and C1-C4 alkoxy;
each R⁷ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members;
X is selected from —OH, —NH₂, —CN, —NH(CO)(C1-C4 alkyl), C1-C4 alkyl, C1-C4 haloalkyl, and C1-C4 alkoxy;
Y¹, Y², Y³, Y⁴, Y⁵, and Y⁶ are independently selected from N and —CR⁸, wherein R⁸ is selected from H, F, Cl, CN, CH₃, and CF₃; and
n is an integer selected from 1-3. Also disclosed herein is a pharmaceutical composition, comprising a compound of Formula I and/or a stereoisomer, a stable isotope, or a pharmaceutically acceptable salt or solvate thereof disclosed herein and a pharmaceutically acceptable carrier.

Further disclosed herein is a method of inhibiting the activity of RET comprising contacting the protein RET with an effective amount of a compound of Formula I and/or a stereoisomer, a stable isotope, or a pharmaceutically acceptable salt or solvate thereof disclosed herein.

Further disclosed herein is a method of treating a disease treatable by inhibition of ERT in a patient, comprising administering to the patient in recognized need of such treatment, an effective amount of a compound of Formula I and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein and a pharmaceutically acceptable carrier.

Further disclosed herein is a method of treating a cancer in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein, and a pharmaceutically acceptable carrier. In some embodiments, the cancer is selected from lung cancers, thyroid cancers, pancreatic cancers, salivary gland cancers, spitz tumors, colorectal cancers, ovarian cancers, and myeloproliferative cancers.

Further disclosed herein is a use of a compound of Formula I and/or a stereoisomer, a stable isotope, or a pharmaceutically acceptable salt or solvate thereof in preparation of a medication for treating a disease responsive to inhibition of RET, such as a cancer. In some embodiments, the cancer is selected from lung cancers, thyroid cancers, pancreatic cancers, salivary gland cancers, spitz tumors, colorectal cancers, ovarian cancers, and myeloproliferative cancers.

Further disclosed herein are compounds of Formula I and the subgenera of Formula I disclosed herein, as well as pharmaceutically acceptable salts or solvates of these compounds, and all stereoisomers (including diastereoisomers and enantiomers, and isotopically enriched versions thereof (including deuterium substitutions). These compounds can be used to treat conditions responsive to RET inhibition, such as those disclosed herein, and for use in the preparation of a medicament for treating these disorders. The pharmaceutical compositions and methods disclosed herein can also be used with or formulated with a co-therapeutic agent; for example, compounds of Formula I and sub-formula thereof can be used with or formulated with at least one agent selected from inhibitors of and non-RET kinase and other therapeutic agents.

Further disclosed are methods, as well as key intermediate compounds, useful for making the compounds of Formula I as disclosed herein.

As used herein, the following words, phrases and symbols are generally intended to have the meanings as set forth below, except to the extent that the context in which they are used indicates otherwise. The following abbreviations and terms have the indicated meanings throughout.

### DETAILED DESCRIPTION

The following definitions apply unless otherwise provided or apparent from context:

A dash (“-”) that is not between two letters or symbols is used to indicate a point of attachment for a substituent. For example, —CONRₐR_{b} is attached through the carbon atom.

Unless clearly indicated otherwise, use of the terms “a”, “an” and the like refers to one or more.

The term “halogen” or “halo” herein refers to fluorine (F), chlorine (Cl), bromine (Br) or iodine (I). Halogen-substituted groups and moieties, such as alkyl substituted by halogen (haloalkyl) can be mono-, poly-, or per-halogenated. In some embodiments, chloro and fluoro are examples of halo substituents on alkyl or cycloalkyl groups, unless otherwise specified; fluoro, chloro, and bromo are used, for example, on aryl or heteroaryl groups, unless otherwise specified.

The term “heteroatoms” or “hetero atoms” as used herein refers to nitrogen (N) or oxygen (O) or sulfur(S) atoms, such as nitrogen or oxygen, unless otherwise specified.

The term “optional” or “optionally” used herein means that the subsequently described event or circumstance may or may not occur, and that the description includes instances where the event or circumstance occurs and instances in which it does not. For example, “alkyl optionally substituted with X” encompasses both “alkyl without substitution of X” and “alkyl substituted with X.” It will be understood by those skilled in the art, with respect to any group containing one or more substituents, that such groups are not intended to introduce any substitution or substitution patterns that are sterically impractical, synthetically non-feasible and/or inherently unstable in water at room temperature for at least long enough to be administered as a pharmaceutical agent. When multiple substituents are present, the substituents are selected independently unless otherwise indicated, so where 2 or 3 substituents are present, for example, those substituents may be the same or different.

In some embodiments, “substituted with at least one group” refers to one hydrogen on the designated atom or group being replaced with one selection from the indicated group of substituents. In some embodiments, “substituted with at least one group” refers to two hydrogens on the designated atom or group being independently replaced with two selections from the indicated group of substituents. In some embodiments, “substituted with at least one group” refers to three hydrogens on the designated atom or group being independently replaced with three selections from the indicated group of substituents. In some embodiments, “substituted with at least one group” refers to four hydrogens on the designated atom or group being independently replaced with four selections from the indicated group of substituents.

The term “alkyl” herein refers to a hydrocarbon group chosen from linear and branched saturated hydrocarbon groups having up to 18 carbon atoms, such as from 1 to 12, further such as from 1 to 8, even further such as from 1 to 6, carbon atoms. Representative examples of alkyl include, but not limited to, methyl, ethyl, n-propyl, iso-propyl, n-butyl, sec-butyl, iso-butyl, tert-butyl, n-pentyl, iso-pentyl, neopentyl, n-hexyl, 3-methylhexyl, 2,2-dimethylpentyl, 2,3-dimethylpentyl, n-heptyl, n-octyl, n-nonyl, n-decyl, and the like.

Unless indicated specifically, alkyl group can be optionally substituted by one or more substituents in place of hydrogen atoms of the unsubstituted alkyl, such as one, two or three substituents, or 1-4 substituents, up to the number of hydrogens present on the unsubstituted alkyl group. Suitable substituents for alkyl groups, if not otherwise specified, may be selected from halogen, D, CN, oxo, hydroxyl, substituted or unsubstituted C1-C4 alkoxy, substituted or unsubstituted C3-C6 cycloalkyl, substituted or unsubstituted 3-7 membered heterocycloalkyl containing 1 or 2 heteroatoms selected from N, O and S as ring members, substituted or unsubstituted aryl, substituted or unsubstituted heteroaryl containing 1 to 4 heteroatoms selected from N, O and S as ring members, amino, —NH(C1-C4 alkyl), —N(C1-C4 alkyl)₂, —S(═O)₀₋₂(C1-C4 alkyl), —S(═NR)(═O)(C1-C4 alkyl), —C(═O)(C1-C4 alkyl), —C(═NOH)(C1-C4 alkyl), —CO₂H, —CO₂(C1-C4 alkyl), —S(═O)₁₋₂NH₂, —S(═O)₁₋₂NH(C1-C4 alkyl), —S(═O)₁₋₂N(C1-C4 alkyl)₂, —CONH₂, —C(═O)NH(C1-C4 alkyl), —C(═O)N(C1-C4 alkyl)₂, —C(═NOH)NH(C1-C4 alkyl), —OC(═O)(C1-C4 alkyl), —NHC(═O)(C1-C4 alkyl), —NHC(═NOH)(C1-C4 alkyl), —NH(C═O)NH₂, —NHC(═O)O(C1-C4 alkyl), —NHC(═O)NH(C1-C4 alkyl), NHC(═NOH)NH(C1-C4 alkyl), —NHS(═O)₁₋₂(C1-C4 alkyl), —NHS(═O)₁₋₂NH₂, and —NHS(═O)₁₋₂NH(C1-C4 alkyl); wherein the substituents for substituted C1-C4 alkoxy, substituted C3-C6 cycloalkyl, substituted 3-7 membered heterocycloalkyl, substituted aryl, and substituted heteroaryl are up to three groups independently selected from halogen, D, —CN, C1-C4 alkyl, C1-C4 haloalkyl, oxo, hydroxy, C1-C4 alkoxy, amino, —NH(C1-C4 alkyl), and —N(C1-C4 alkyl)₂. In some embodiments, substituents for alkyl groups, unless otherwise specified, are selected, for example, from halogen, CN, oxo, hydroxy, C1-C4 alkoxy, C3-C6 cycloalkyl, phenyl, amino, —NH(C1-C4 alkyl), —N(C1-C4 alkyl)₂, C1-C4 alkylthio, C1-C4 alkylsulfonyl, —C(═O)(C1-C4 alkyl), —CO₂H, —CO₂ (C1-C4 alkyl), —OC(═O)(C1-C4 alkyl), —NHC(═O)(C1-C4 alkyl), and —NHC(═O)O(C1-C4 alkyl).

The term “alkoxy” herein refers to a straight or branched alkyl group comprising from 1 to 18 carbon atoms attached through an oxygen bridge such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Typically, alkoxy groups comprise from 1 to 6 carbon atoms, such as 1 to 4 carbon atoms, attached through the oxygen bridge.

Unless indicated specifically, alkoxy group can be optionally substituted by one or more substituents in place of hydrogen atoms of the unsubstituted alkyl portion of the alkoxy, such as one, two or three substituents, or 1-4 substituents, up to the number of hydrogens present on the unsubstituted alkoxy group. Unless otherwise specified, suitable substituents are selected, for example, from the substituents listed above for alkyl groups, except that hydroxyl and amino are not normally present on the carbon that is directly attached to the oxygen of the substituted alkyl-O group.

The term “alkenyl” herein refers to a hydrocarbon group selected from linear and branched hydrocarbon groups, comprising at least one C═C double bond and from 2 to 18, such as from 2 to 6, carbon atoms. Examples of the alkenyl group may be selected from ethenyl or vinyl (—CH═CH₂), prop-1-enyl (—CH—CHCH₃), prop-2-enyl (—CH₂CH═CH₂), 2-methylprop-1-enyl, buta-1-enyl, buta-2-enyl, buta-3-enyl, buta-1,3-dienyl, 2-methylbuta-1,3-diene, hex-1-enyl, hex-2-enyl, hex-3-enyl, hex-4-enyl, and hexa-1,3-dienyl groups. The point of attachment can be on the unsaturated carbon or saturated carbon.

Unless indicated specifically, alkenyl group can be optionally substituted by one or more substituents in place of hydrogen atoms of the unsubstituted alkenyl, such as one, two or three substituents, or 1-4 substituents, up to the number of hydrogens present on the unsubstituted alkenyl group. Unless otherwise specified, suitable substituents are selected, for example, from the substituents listed above for alkyl groups.

The term “alkynyl” herein refers to a hydrocarbon group selected from linear and branched hydrocarbon groups, comprising at least one-C═C-triple bond and from 2 to 18, such as from 2 to 6 carbon atoms. Examples of the alkynyl group include ethynyl (—C═CH), 1-propynyl (—C═CCH₃), 2-propynyl (propargyl, —CH₂C≡CH), 1-butynyl, 2-butynyl, and 3-butynyl groups. The point of attachment can be on the unsaturated carbon or saturated carbon.

Unless indicated specifically, alkynyl group can be optionally substituted by one or more substituents in place of hydrogen atoms of the unsubstituted alkynyl, such as one, two or three substituents, or 1-4 substituents, up to the number of hydrogens present on the unsubstituted alkynyl group. Unless otherwise specified, suitable substituents are selected, for example, from the substituents listed above for alkyl groups.

The term “alkylene” refers to a divalent alkyl group comprising from 1 to 10 carbon atoms, and two open valences to attach to other molecular components. The two molecular components attached to an alkylene can be on the same carbon atom or on different carbon atoms; thus for example propylene is a 3-carbon alkylene that can be 1,1-disubstituted, 1,2-disubstituted or 1,3-disubstituted. Unless otherwise specified, alkylene refers to moieties comprising from 1 to 6 carbon atoms, such as from 1 to 4 carbon atoms. Examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents; unless otherwise specified, suitable substituents are selected, for example, from the substituents listed above for alkyl groups.

Unless indicated specifically, alkylenyl group can be optionally substituted by one or more substituents in place of hydrogen atoms of the unsubstituted alkylenyl, such as one, two or three substituents, or 1-4 substituents, up to the number of hydrogens present on the unsubstituted alkylenyl group. Unless otherwise specified, suitable substituents are selected, for example, from the substituents listed above for alkyl groups.

Similarly, “alkenylene” and “alkynylene” refer to alkylene groups comprising a double bond or a triple bond, respectively; they are, for example, 2-6 such as 2-4 carbon atoms in length, and can be substituted as discussed above for alkylene groups.

The term “haloalkyl” refers to an alkyl as defined herein, which is substituted by one or more halo groups as defined herein. Unless otherwise specified, the alkyl portion of the haloalkyl comprises 1˜4 carbon atoms. The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. The polyhaloalkyl comprises, for example, up to 6, or 4, or 3, or 2 halo groups. Examples of haloalkyl include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl. In some embodiments, the haloalkyl groups, unless specified otherwise, include monofluoro-, difluoro- and trifluoro-substituted methyl and ethyl groups, e.g. —CF₃, —CF₂H, —CFH₂ and —CH₂CF₃.

Unless indicated specifically, haloalkyl group can be optionally substituted by one or more substituents in place of hydrogen atoms of the unsubstituted haloalkyl, such as one, two or three substituents, or 1-4 substituents, up to the number of hydrogens present on the unsubstituted haloalkyl group. Unless otherwise specified, suitable substituents are selected, for example, from the substituents listed above for alkyl groups.

As used herein, the term “haloalkoxy” refers to haloalkyl-O—, wherein haloalkyl is defined above. Examples of haloalkoxy include, but are not limited to, fluoromethoxy, difluoromethoxy, trifluoromethoxy, trichloromethoxy, 2-chloroethoxy, 2,2,2-trifluoroethoxy, 1,1,1,3,3,3-hexafluoro-2-propoxy, and the like. In some embodiments, haloalkyloxy groups comprise 1-4 carbon atoms, and up to three halogens, e.g., monofluoro, difluoro and trifluoro substituted methoxy groups and ethoxy groups.

Unless indicated specifically, haloalkoxy group can be optionally substituted by one or more substituents in place of hydrogen atoms of the unsubstituted alkyl portion of the haloalkoxy, such as one, two or three substituents, or 1-4 substituents, up to the number of hydrogens present on the unsubstituted haloalkoxy group. Unless otherwise specified, suitable substituents are selected, for example, from the substituents listed above for alkyl groups, except that hydroxyl and amino are not normally present on the carbon that is directly attached to the oxygen of the substituted haloalkyl-O group.

The term “cycloalkyl” herein refers to a hydrocarbon group selected from saturated and partially unsaturated cyclic hydrocarbon groups comprising from 3 to 20 carbon atoms, such as monocyclic and polycyclic (e.g., bicyclic and tricyclic, admantanyl and spirocycloalkly) groups. Monocycloalkyl groups are cyclic hydrocarbon groups comprising from 3 to 20 carbon atoms, such as from 3 to 8 carbon atoms. Examples of monocyclic cycloalkyl include, but are not limited to, cyclopropyl, cyclobutyl, cyclopentyl, cyclohexyl, cycloheptyl, cyclooctyl, cyclodecanyl, cyclodocecanyl, and cyclohexenyl. Bicycloalkyl groups include bridged bicycloalkyl, fused bicycloalkyl and spirocycloalkyls. Bridged bicycloalkyl contains a monocyclic cycloalkyl ring where two non-adjacent carbon atoms of the monocyclic ring are linked by an alkylene bridge of one to three additional carbon atoms (i.e. a bridging group of the form —(CH₂)ₙ—, wherein n is 1, 2, or 3). Examples of bridged bicycloalkyl include, but are not limited to, bicyclo[2.2.1]heptenes, bicyclo[3.1.1]heptanes, bicyclo[2.2.1]heptanes, bicyclo[2.2.2]octane, bicyclo[3.2.2]nonane, bicyclo[3.3.1]nonane, and bicycle[4.2.1]nonane. Fused bicycloalkyl contains a monocyclic cycloalkyl ring fused to either a phenyl, a monocyclic cycloalkyl, or a monocyclic heteroaryl. Examples of fused bicycloalkyl include, but are not limited to, bicyclo[4.2.0]octa-1,3,5-triene, 2,3-dihydro-1H-indene, 6,7-dihydro-5H-cyclopenta[b]pyridine, 5,6-dihydro-4H-cyclopenta[b]thiophene, and decahydronaphthalene. Spirocycloalkyl contains two monocyclic ring systems that share a carbon atom forming a biclyclic ring system. Examples of spirocycloalkyls include, but are not limited to,

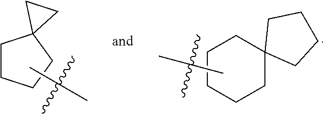

Bicyclic cycloalkyl groups comprise, for example, from 7 to 12 carbon atoms. Monocycloalkyl or bicycloalkyl is attached to the parent molecular moiety through any carbon atom contained within the cycloalkyl ring. Tricycloalkyl groups include bridged tricycloalkyl as used herein referring to 1) a bridged bicycloalkyl ring where two non-adjacent carbon atoms of the bridged bicycloalkyl ring are linked by an alkylene bridge of one to three additional carbon atoms (i.e. a bridging group of the form —(CH₂)ₙ—, wherein n is 1, 2, or 3), or 2) a fused bicycloalkyl ring where two unshared ring atoms on each ring are linked by an alkylene bridge of one to three additional carbon atoms (i.e. a bridging group of the form —(CH₂)ₙ—, wherein n is 1, 2, or 3), wherein “a fused bicycloalkyl ring” refers to a monocycloalkyl ring fused to a monocycloalkyl ring. Examples of bridged tricycloalkyl groups include, but are not limited to, admantanyl

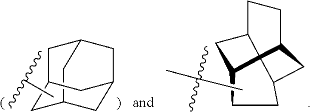

Bridged tricycloalkyl, as used hererin, is appended to the parent molecular moiety through any ring atom. The ring atom disclosed herein refers to the carbon atom on the ring skeleton. The cycloalkyl may be saturated or comprise at least one double bond (i.e., partially unsaturated), but is not fully conjugated, and is not aromatic, as aromatic is defined herein. The cycloalkyl may be substituted with at least one hetero atom selected, for example, from O, S, and N.

Unless indicated specifically, cycloalkyl group can be optionally substituted by one or more substituents in place of hydrogen atoms of the unsubstituted cycloalkyl, such as one, two or three substituents, or 1-4 substituents, up to the number of hydrogens present on the unsubstituted cycloalkyl group. In some embodiments, a substituted cycloalkyl comprises 1-4 such as 1-2 substituents. Unless otherwise specified, suitable substituents are selected, for example, from the substituents listed above for alkyl groups.

The term “cycloalkylidenyl” or “cycloalkylidene ring” disclosed herein refers to a divalent cycloalkane ring attached via the same carbon atom of the cycloalkane ring by removal of two hydrogen atoms from the same carbon atoms. Examples of cycloakylidenyl rings include, but are not limited to, cyclopropylidenyl, cyclobutylidenyl, cyclopentylidenyl, and cyclohexylidenyl. It can be represented in illustrative fashion by the following structure in which n is 1, 2, 3, 4, or 5.

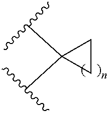

The term “heterocycloalkyl,” “heterocyclyl,” or “heterocyclic” disclosed herein refers to “cycloalkyl” as defined above with at least one ring carbon atom being replaced by a heteroatom independently selected from O, N, and S. Heterocyclyl comprises, for example, 1, 2, 3, or 4 heteroatoms, and the N, C or S can independently be oxidized in the cyclic ring system. The N atom can further be substituted to form tertiary amine or ammonium salts. The point of attachment of heterocyclyl can be on the heteroatom or carbon. “Heterocyclyl” herein also refers to a 5- to 7-membered saturated or partially unsaturated carbocyclic ring comprising at least one heteroatom selected, for example, from N, O, and S (heterocyclic ring) fused with 5-, 6-, and/or 7-membered cycloalkyl, heterocyclic or carbocyclic aromatic ring, provided that the point of attachment is at the heterocyclic ring when the heterocyclic ring is fused with a carbocyclic aromatic ring, and that the point of attachment can be at the cycloalkyl or heterocyclic ring when the heterocyclic ring is fused with cycloalkyl. “Heterocyclyl” herein also refers to an aliphatic spirocyclic ring comprising at least one heteroatom selected, for example, from N, O, and S. The rings may be saturated or have at least one double bond (i.e., partially unsaturated). The heterocyclyl may be substituted with, for example, oxo. The point of the attachment may be carbon or heteroatom. A heterocyclyl is not a heteroaryl as defined herein.

Examples of the heterocycle include, but not limited to, pyrrolidinyl, imidazolidinyl, pyrazolidinyl, piperidinyl, piperidinyl, piperazinyl, pyranyl, morpholinyl, oxiranyl, aziridinyl, thiiranyl, azetidinyl, oxetanyl, thietanyl, dithietanyl, dihydropyridinyl, tetrahydropyridinyl, thiomorpholinyl, thioxanyl, homopiperazinyl, homopiperidinyl, azepanyl, oxepanyl, thiepanyl, oxathianyl, dioxepanyl, oxathiepanyl, oxaazepanyl, dithiepanyl, thiazepanyl and diazepane, dithianyl, azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, indolinyl, dioxanyl, pyrazolinyl, dithianyl, dithiolanyl, pyrazolidinyl, imidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycles also include ring systems substituted with one or more oxo moieties, such as piperidinyl N-oxide, morpholinyl-N-oxide, 1-oxo-1-thiomorpholinyl, 1,1-dioxo-1-thiomorpholinyl,

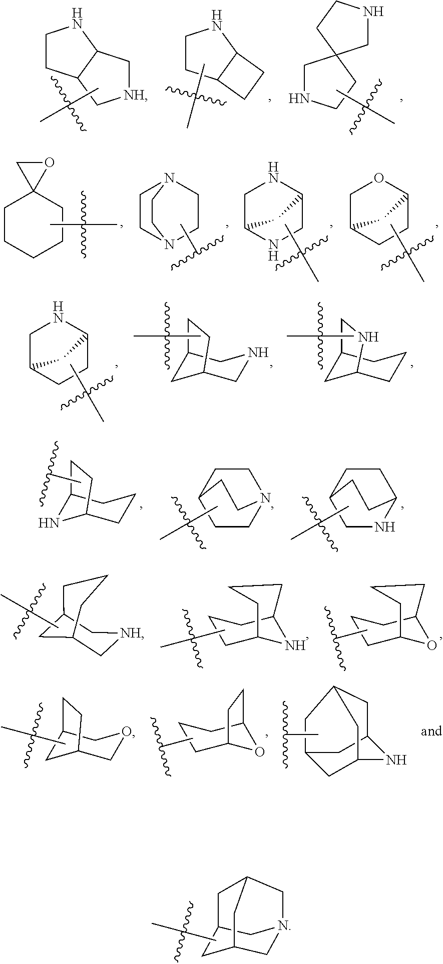

Unless indicated specifically, heterocyclyl group can be optionally substituted by one or more substituents in place of hydrogen atoms of the unsubstituted heterocyclyl, such as one, two or three substituents, or 1-4 substituents, up to the number of hydrogens present on the unsubstituted heterocyclyl group. In some embodiments, a substituted heterocycloalkyl comprises 1˜4 such as 1-2 substituents. Unless otherwise specified, suitable substituents are selected, for example, from the substituents listed above for alkyl groups.

The term “aryl” refers to an aromatic hydrocarbon group comprising 5-15 carbon atoms in the ring portion. In some embodiments, aryl refers to a group selected from 5- and 6-membered carbocyclic aromatic rings, for example, phenyl; bicyclic ring systems such as 7 to 12 membered bicyclic ring systems wherein at least one ring is carbocyclic and aromatic, selected, for example, from naphthalene, indane, and 1,2,3,4-tetrahydroquinoline; and tricyclic ring systems such as 10 to 15 membered tricyclic ring systems, wherein at least one ring is carbocyclic and aromatic, for example, fluorene.

In some embodiments, the aryl group is selected from 5- and 6-membered carbocyclic aromatic rings fused to a 5- to 7-membered cycloalkyl or heterocyclic ring (as defined in “heterocyclyl” or “heterocyclic” below) optionally comprising at least one heteroatom selected, for example, from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring (e.g., a heteroaryl as defined below), the resulting ring system is heteroaryl, not aryl, as defined herein.

Unless indicated specifically, aryl group can be optionally substituted by one or more substituents in place of hydrogen atoms of the unsubstituted aryl, such as one, two or three substituents, or 1-4 substituents, up to the number of hydrogens present on the unsubstituted aryl group. In some embodiments, a substituted aryl group comprises 1-5 substituents. Unless otherwise specified, suitable substituents are selected, for example, from the substituents listed above for alkyl groups.

The term “heteroaryl” herein refers to a group selected from 5- to 7-membered aromatic, monocyclic rings comprising at least one heteroatom, for example, from 1 to 4, or, in some embodiments, from 1 to 3, heteroatoms, selected, for example, from N, O, and S, with the remaining ring atoms being carbon; 8- to 12-membered bicyclic rings comprising at least one heteroatom, for example, from 1 to 4, or, in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in the aromatic ring, and with the point of attachment being on any ring and being on either carbon or the heteroatom; and 11- to 14-membered tricyclic rings comprising at least one heteroatom, for example, from 1 to 4, or in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in an aromatic ring, and with the point of attachment being on any ring.

In some embodiments, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

In some embodiments, the heteroaryl group includes a 5- to 7-membered heterocyclic aromatic ring fused to a 5- to 7-membered aryl ring. For such fused, bicyclic heteroaryl ring systems wherein only one of the rings comprises at least one heteroatom, the point of attachment may be at the heteroaromatic ring or at the aryl ring. Non-limiting examples include quinolinyl and quinazolinyl.

In some embodiments, the heteroaryl group includes a 5- to 7-membered heterocyclic aromatic ring fused to another 5- to 7-membered heterocyclic aromatic ring. Non-limiting examples include 1H-pyrazolo[3,4-b]pyridinyl and 1H-pyrrolo[2,3-b]pyridinyl.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, pyridyl, cinnolinyl, pyrazinyl, pyrimidinyl, imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrimidinyl, pyrrolyl, triazolyl, quinolinyl, isoquinolinyl, pyrazolyl, pyrrolopyridinyl (such as 1H-pyrrolo[2,3-b]pyridin-3-yl), pyrazolopyridinyl (such as 1H-pyrazolo[3,4-b]pyridin-3-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

Unless indicated specifically, heteroaryl group can be optionally substituted by one or more substituents in place of hydrogen atoms of the unsubstituted heteroaryl, such as one, two or three substituents, or 1-4 substituents, up to the number of hydrogens present on the unsubstituted heteroaryl group. In some embodiments, a substituted heteroaryl group comprises 1, 2 or 3 substituents. Unless otherwise specified, suitable substituents are selected, for example, from the substituents listed above for alkyl groups.

Compounds disclosed herein may contain an asymmetric center and may thus exist as enantiomers. Where the compounds disclosed herein possess two or more asymmetric centers, they may additionally exist as diastereomers. Enantiomers and diastereomers fall within the broader class of stereoisomers. It is well-known in the art how to prepare optically active forms, such as by resolution of materials or by asymmetric synthesis. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When the compounds disclosed herein contain olefinic double bonds, unless specified otherwise, such double bonds are meant to include both E and Z geometric isomers. “A pharmaceutically acceptable salt” includes, but is not limited to, salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, aluminum, lithium, and ammonium.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating”, “treat”, “treatment” or “alleviation” refers to administering at least one compound and/or at least one stereoisomer thereof, if any, at least one stable isotope thereof, or at least one pharmaceutically acceptable salt thereof disclosed herein to a subject in recognized need thereof that has, for example, cancer.

The term “effective amount” refers to an amount of at least one compound and/or at least one stereoisomer thereof, if any, at least one stable isotope thereof, or at least one pharmaceutically acceptable salt thereof disclosed herein effective to “treat,” as defined above, a disease or disorder in a subject.

The term “RET-associated disease”, “RET-associated disorder”, “RET-associated cancer”, “diseases and disorders modulated by RET”, or “aberrant RET activity” refers to disease, disorder, or cancer associated with or having a dysregulation of RET gene. The dysregulation of a RET gene is caused by RET gene mutation that consists of, for example, a RET gene translocation resulting in the expression of a fusion protein, a deletion in a RET gene resulting in the expression of a RET protein that includes a deletion of at least one amino acid as compared to the wild-type RET protein, a mutation in a RET gene that results in the expression of a RET protein with one or more mutations, an alternative spliced version of a RET mRNA that results in a RET protein having a deletion of at least one amino acid in the RET protein, or a RET gene amplification that results in overexpression of a RET gene in a cell leading to a pathogenic increase in the activity of a kinase domain of a RET protein or a constitutively active kinase domain of a RET protein in cell. For example, at least 12 different fusion variants have been identified, with KIF5B-RET being the most common in NSCLCs, and CCDC6 and NCOA4 being most common in PTCs Example of RET point mutations are, not limited to, M918T, G810R, V804L and V804M (Drilon, A. et al. Nat. Rev. Clin. Oncol., 2018, 15, 151-167). Examples of RET-associated diseases or disorders include, but are not limited to, cancers and gastrointestinal disorders such as irritable bowel syndrome.

Various embodiments are disclosed herein. It will be recognized that features specified in each embodiment may be combined with other specified features to provide further embodiments of the present disclosure. The following enumerated embodiments are representative of the present disclosure.

Embodiment 1. A compound of Formula I:

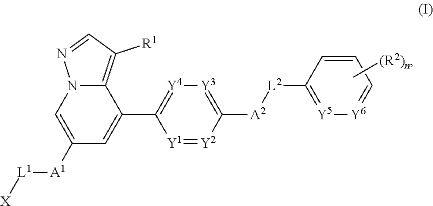

and/or stereoisomers, stable isotopes, or pharmaceutically acceptable salts or solvates thereof, wherein:
A¹ is a cyclic group selected from phenyl and 5- to 6-membered heteroaryl, wherein the 5- to 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl;
A² is a group selected from:

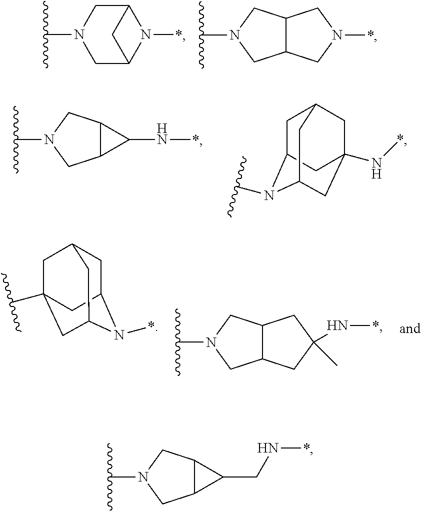

wherein the bond marked with an asterisk (*) represents the bond to L² of Formula I;
L¹ is a group selected from:

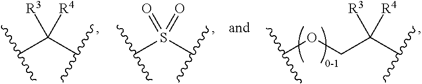

wherein R³ and R⁴ are each independently selected from H and C1-C6 alkyl, wherein the C1-C6 alkyl is optionally substituted with 1-3 groups independently selected from CN, halo, OH, and C1-C6 alkoxy; or wherein R³ and R⁴ are taken together to form a group selected from oxo, 3- to 6-membered cycloalkyl, and 5- to 6-membered heterocycles containing 1-2 heteroatoms independently selected from N, O, and S as ring members;
L² is a group selected from CO, SO₁₋₂, C1-C6 alkylenyl, and C1-C6 haloalkylenyl:
R¹ is selected from —CN, ethynyl, halo, —CF₃, —CH₃, —CH₂CH₃, cyclopropyl, —CH₂CN, and —CH(CN)CH₃;
each R² is independently selected from halo, —OR⁵, —N(R⁵)₂, —CN, C1-C6 alkyl, C1-C6 haloalkyl, C3-C6 cycloalkyl, —OC(O)R⁵, —CO₂R⁵, —C(O)N(R⁵)₂, —C(═NR⁶)N(R⁵)₂, —C(O)R⁵, —S(O)₀₋₂R⁷, —S(O)(═NR⁶) R⁷, —S(O)₁₋₂N(R⁵)₂, —N(R⁵)C(O)R⁷, —N(R⁵)C(═NR⁶) R⁷, —N(R⁵)S(O)₁₋₂R⁷, —N(R⁵)C(O)N(R⁵)₂, —N(R⁵)C(═NR⁶)N(R⁵)₂, —N(R⁵)S(O)₁₋₂N(R⁵)₂, and —N(R⁵)CO₂R⁷, wherein:
each R⁵ is independently selected from H, C1-C6 alkyl, C3-C6 cycloalkyl, C1-C6 haloalkyl, aryl, saturated and unsaturated 4-7 membered heterocyclyl containing 1-2 heteroatoms selected from N, O, and S as ring members, and heteroaryl containing 1-4 heteroatoms selected from N, O, and S as ring members;
R⁶ is independently selected from H, —CN, —OH, C1-C4 alkyl, and C1-C4 alkoxy;
R⁷ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members;
X is selected from —OH, —NH₂, —CN, —NH(CO)(C1-C4 alkyl), C1-C4 alkyl, C1-C4 haloalkyl, and C1-C4 alkoxy;
Y¹, Y², Y³, Y⁴, Y⁵, and Y⁶ are independently selected from N and —CR⁸, wherein R⁸ is selected from H, F, Cl, CN, CH₃, and CF₃; and
n is an integer selected from 1-3.

Embodiment 2. The compound of Embodiment 1, and/or a stereoisomer, a stable isotope, or a pharmaceutically acceptable salt or solvate thereof, wherein A¹ is a cyclic group selected from phenyl, pyridine, pyrimidine, pyrazine, pyridazine, pyrazole, thiophene, thiazole, and oxazole, and wherein the cyclic group is optionally substituted with 1-3 groups independently selected from halogen, CN, C1-C4 alkyl, and C1-C4 haloalkyl.

Embodiment 3. The compound of Embodiment 1 or Embodiment 2, and/or a stereoisomer, a stable isotope, or a pharmaceutically acceptable salt or solvate thereof, wherein A¹ is a cyclic group selected from:

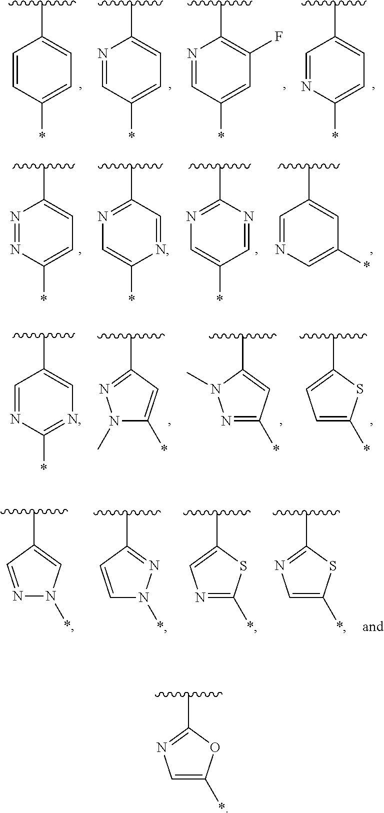

wherein the bond marked with an asterisk (*) represents the bond to L¹ of Formula I.

Embodiment 4. The compound of Embodiment 1, and/or a stereoisomer, a stable isotope, or a pharmaceutically acceptable salt or solvate thereof, wherein A¹ is a cyclic group selected from 5- to 6-membered heteroaryl, wherein the 5- to 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl.

Embodiment 5. The compound of Embodiment 1 or Embodiment 4, and/or a stereoisomer, a stable isotope, or a pharmaceutically acceptable salt or solvate thereof, wherein A¹ is a cyclic group selected from pyridine, pyrimidine, pyrazine, pyridazine, pyrazole, thiophene, thiazole, and oxazole, and wherein the cyclic group is optionally substituted with 1-3 groups independently selected from halogen, CN, C1-C4 alkyl, and C1-C4 haloalkyl.

Embodiment 6. The compound of any one of Embodiments 1-5, and/or a stereoisomer, a stable isotope, or a pharmaceutically acceptable salt or solvate thereof, wherein A¹ is a cyclic group selected from:

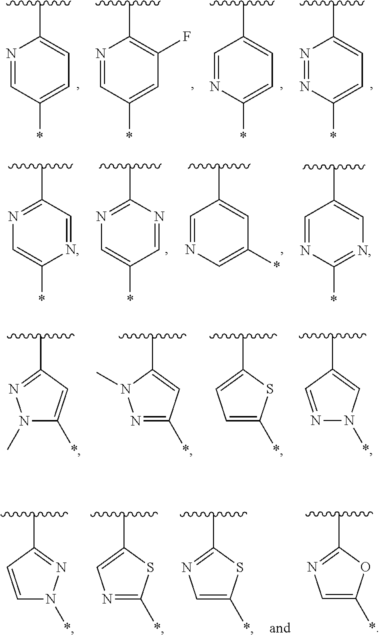

wherein the bond marked with an asterisk (*) represents the bond to L¹ of Formula I.

Embodiment 7. The compound of Embodiment 1, and/or a stereoisomer, a stable isotope, or a pharmaceutically acceptable salt or solvate thereof, wherein A¹ is a cyclic group selected from 6-membered heteroaryl, wherein the 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl.

Embodiment 8. The compound of Embodiment 1 or Embodiment 7, and/or a stereoisomer, a stable isotope, or a pharmaceutically acceptable salt or solvate thereof, wherein A¹ is a cyclic group selected from pyridine, pyrimidine, pyrazine, and pyridazine, and wherein the cyclic group is optionally substituted with 1-3 groups independently selected from halogen, CN, C1-C4 alkyl, and C1-C4 haloalkyl.

Embodiment 9. The compound of any one of Embodiments 1-8, and/or a stereoisomer, a stable isotope, or a pharmaceutically acceptable salt or solvate thereof, wherein A¹ is a cyclic group selected from:

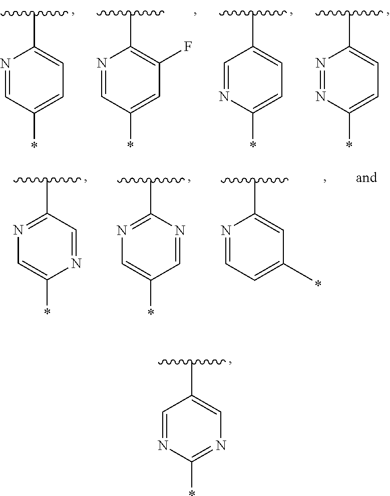

wherein the bond marked with an asterisk (*) represents the bond to L¹ of Formula I.

Embodiment 10. The compound of any one of Embodiments 1-9, and/or a stereoisomer, a stable isotope, or a pharmaceutically acceptable salt or solvate thereof, wherein A² is a group selected from:

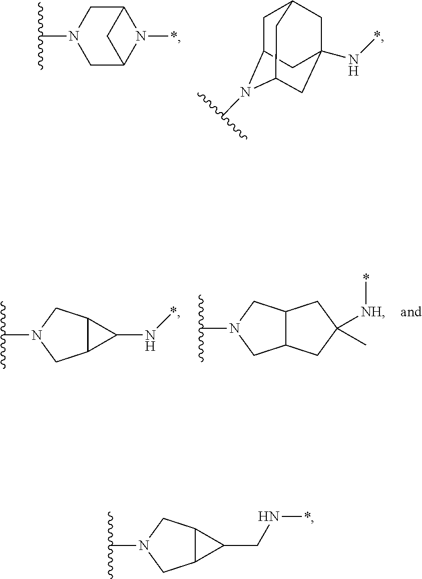

wherein the bond marked with an asterisk (*) represents the bond to L² of Formula I.

Embodiment 11. The compound of any one of Embodiments 1-10, and/or a stereoisomer, a stable isotope, or a pharmaceutically acceptable salt or solvate thereof, wherein A² is

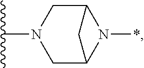

wherein the bond marked with an asterisk (*) represents the bond to L² of Formula I.

Embodiment 12. The compound of any one of Embodiments 1-11, and/or a stereoisomer, a stable isotope, or a pharmaceutically acceptable salt or solvate thereof, wherein L¹ is a group selected from:

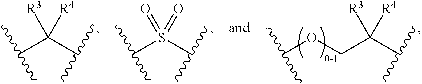

R³ and R⁴ are independently selected from H and CH₃; or R³ and R⁴ are taken together to form a group selected from oxo and 3- to 4-membered cycloalkyl.

Embodiment 13. The compound of any one of Embodiments 1-12, and/or a stereoisomer, a stable isotope, or a pharmaceutically acceptable salt or solvate thereof, wherein L¹ is a group selected from:

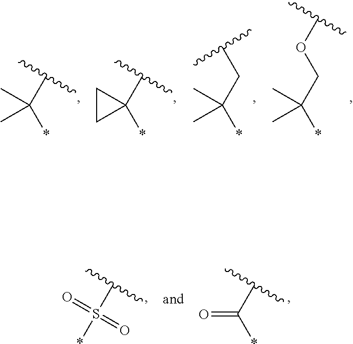

wherein the bond marked with an asterisk (*) represents the bond to X of Formula I.

Embodiment 14. The compound of any one of Embodiments 1-13, and/or a stereoisomer, a stable isotope, or a pharmaceutically acceptable salt or solvate thereof, wherein L¹ is

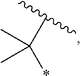 [SMILES: *C(*)(C)C]

wherein the bond marked with an asterisk (*) represents the bond to X of Formula I.

Embodiment 15. The compound of any one of Embodiments 1-14, and/or a stereoisomer, a stable isotope, or a pharmaceutically acceptable salt or solvate thereof, wherein L² is a group selected from CO and C1-C6 alkylenyl.

Embodiment 16. The compound of any one of Embodiments 1-15, and/or a stereoisomer, a stable isotope, or a pharmaceutically acceptable salt or solvate thereof, wherein L² is a group selected from CO and —CH₂—.

Embodiment 17. The compound of any one of Embodiments 1-16, and/or a stereoisomer, a stable isotope, or a pharmaceutically acceptable salt or solvate thereof, wherein L² is —CH₂—.

Embodiment 18. The compound of any one of Embodiments 1-17, and/or a stereoisomer, a stable isotope, or a pharmaceutically acceptable salt or solvate thereof, wherein R¹ is —CN.

Embodiment 19. The compound of any one of Embodiments 1-18, and/or a stereoisomer, a stable isotope, or a pharmaceutically acceptable salt or solvate thereof, wherein each R² is independently selected from halogen, —OH, —CN, C1-C6 alkyl, C1-C6 haloalkyl, and C1-C6 alkoxy.

Embodiment 20. The compound of any one of Embodiments 1-19, and/or a stereoisomer, a stable isotope, or a pharmaceutically acceptable salt or solvate thereof, wherein each R² is independently selected from halogen and C1-C6 alkoxy.

Embodiment 21. The compound of any one of Embodiments 1-20, and/or a stereoisomer, a stable isotope, or a pharmaceutically acceptable salt or solvate thereof, wherein each R² is —OCH₃.

Embodiment 22. The compound of any one of Embodiments 1-21, and/or a stereoisomer, a stable isotope, or a pharmaceutically acceptable salt or solvate thereof, wherein X is selected from —OH, —NH₂, —NH(CO)CH₃, and —CH₂CH₃.

Embodiment 23. The compound of any one of Embodiments 1-22, and/or a stereoisomer, a stable isotope, or a pharmaceutically acceptable salt or solvate thereof, wherein X is selected from —OH and —NH₂.

Embodiment 24. The compound of any one of Embodiments 1-23, and/or a stereoisomer, a stable isotope, or a pharmaceutically acceptable salt or solvate thereof, wherein X is —OH.

Embodiment 25. The compound of any one of Embodiments 1-24, and/or a stereoisomer, a stable isotope, or a pharmaceutically acceptable salt or solvate thereof, wherein Y² is N, and Y¹, Y³, and Y⁴ are independently selected from —CR⁸, wherein R⁸ is selected from H, F, Cl, CN, CH₃, and CF₃.

Embodiment 26. The compound of any one of Embodiments 1-24, and/or a stereoisomer, a stable isotope, or a pharmaceutically acceptable salt or solvate thereof, wherein Y² and Y⁴ are N, and Y¹ and Y³ are independently selected from —CR⁸, wherein R⁸ is selected from H, F, Cl, CN, CH₃, and CF₃.

Embodiment 27. The compound of any one of Embodiments 1-24, and/or a stereoisomer, a stable isotope, or a pharmaceutically acceptable salt or solvate thereof, wherein Y¹ and Y³ are N, and Y² and Y⁴ are independently selected from —CR⁸, wherein R⁸ is selected from H, F, Cl, CN, CH₃, and CF₃.

Embodiment 28. The compound of any one of Embodiments 1-27, and/or a stereoisomer, a stable isotope, or a pharmaceutically acceptable salt or solvate thereof, wherein Y⁵ is N and Y⁶ is selected from —CR⁸, wherein R⁸ is selected from H, F, Cl, CN, CH₃, and CF₃.

Embodiment 29. The compound of any one of Embodiments 1-27, and/or a stereoisomer, a stable isotope, or a pharmaceutically acceptable salt or solvate thereof, wherein Y⁶ is N and Y⁵ is selected from —CR⁸, wherein R⁸ is selected from H, F, Cl, CN, CH₃, and CF₃.

Embodiment 30. The compound of any one of Embodiments 1-29, and/or a stereoisomer, a stable isotope, or a pharmaceutically acceptable salt or solvate thereof, wherein R⁸ is H.

Embodiment 31. The compound of any one of Embodiments 1-30, and/or a stereoisomer, a stable isotope, or a pharmaceutically acceptable salt or solvate thereof, wherein n is 1.

Embodiment 32. The compound of Embodiment 1, wherein the compound is selected from compounds of Formula IA:

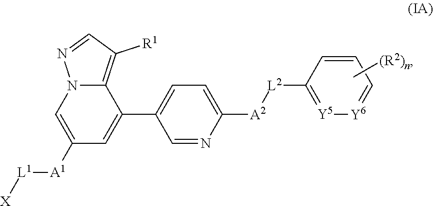

and/or a stereoisomer, a stable isotope, or a pharmaceutically acceptable salt or solvate thereof, wherein A¹, A², L¹, L², R¹, R², X, Y⁵, Y⁶, and n are the same as those defined in Embodiment 1.

Embodiment 33. The compound of Embodiment 1, wherein the compound is selected from compounds of Formula IB:

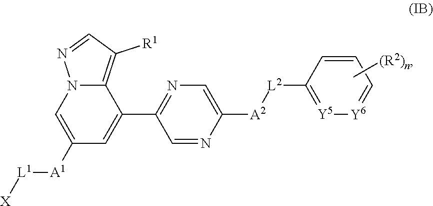

and/or a stereoisomer, a stable isotope, or a pharmaceutically acceptable salt or solvate thereof, wherein A¹, A², L¹, L², R¹, R², X, Y⁵, Y⁶, and n are the same as those defined in Embodiment 1.

Embodiment 34. The compound of Embodiment 1, wherein the compound is selected from compounds of Formula IC:

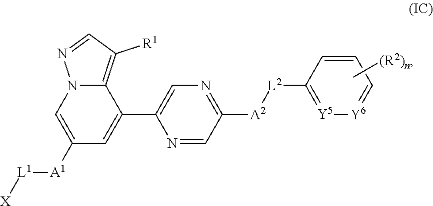

and/or a stereoisomer, a stable isotope, or a pharmaceutically acceptable salt or solvate thereof, wherein A¹, A², L¹, L², R¹, R², X, Y⁵, Y⁶, and n are the same as those defined in Embodiment 1.

Embodiment 35. The compound of Embodiment 1, wherein the compound is selected from compounds of Formula ID:

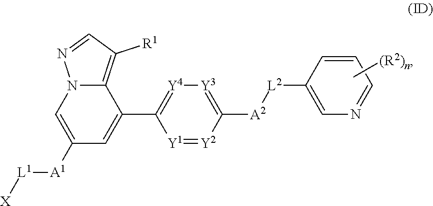

and/or a stereoisomer, a stable isotope, or a pharmaceutically acceptable salt or solvate thereof, wherein A¹, A², L¹, L², R¹, R², X, Y¹, Y², Y³, Y⁴, and n are the same as those defined in Embodiment 1.

Embodiment 36. The compound of Embodiment 1, wherein the compound is selected from compounds of Formula IE:

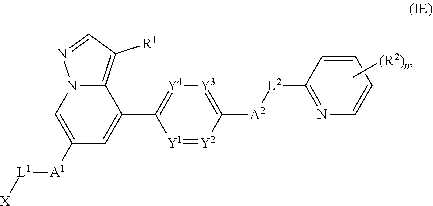

and/or a stereoisomer, a stable isotope, or a pharmaceutically acceptable salt or solvate thereof, wherein A¹, A², L¹, L², R¹, R², X, Y¹, Y², Y³, Y⁴, and n are the same as those defined in Embodiment 1.

Embodiment 37. The compound of Embodiment 1, wherein the compound is selected from compounds of Formula IIA:

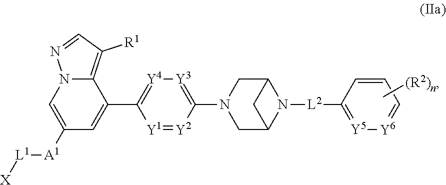

and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in Embodiment 1.

Embodiment 38. The compound of Embodiment 1, wherein the compound is selected from compounds of Formula IIB:

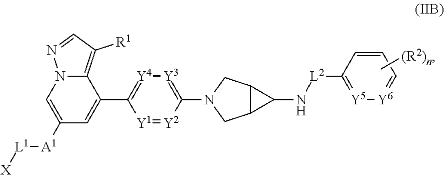

and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in Embodiment 1.

Embodiment 39. The compound of Embodiment 1, wherein the compound is selected from compounds of Formula IIC:

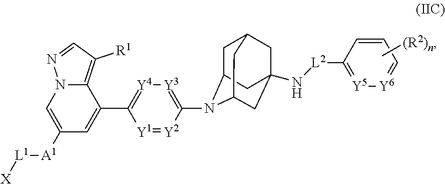

and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in Embodiment 1.

Embodiment 40. The compound of Embodiment 1, wherein the compound is selected from compounds of Formula IID:

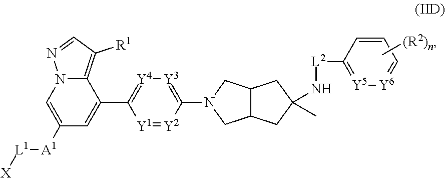

and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in Embodiment 1.

Embodiment 41. The compound of Embodiment 1, wherein the compound is selected from compounds of Formula IIE:

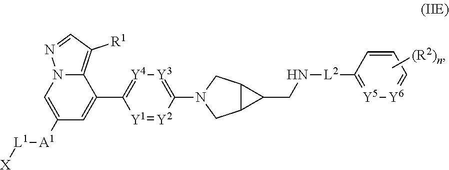

and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in Embodiment 1.

Embodiment 42. A compound selected from the following compounds, and/or a stereoisomer, a stable isotope, or a pharmaceutically acceptable salt or solvate thereof:

| Example # | Structure | Chemical Name |
|---|---|---|
| 1 | 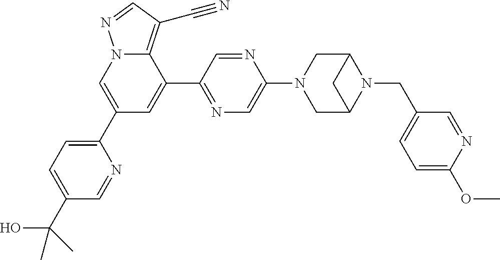 [SMILES: COc1ccc(CN2C3CC2CN(c2cnc(-c4cc(-c5ccc(C(C)(C)O)cn5)cn5ncc(C#N)c45)cn2)C3)cn1] | 6-(5-(2-hydroxypropan-2-yl)pyridin-2-yl)-4- (5-(6-((6-methoxypyridin-3-yl)methyl)-3,6- diazabicyclo[3.1.1]heptan-3-yl)pyrazin-2- yl)pyrazolo[1,5-a]pyridine-3-carbonitrile, |
| 2 | 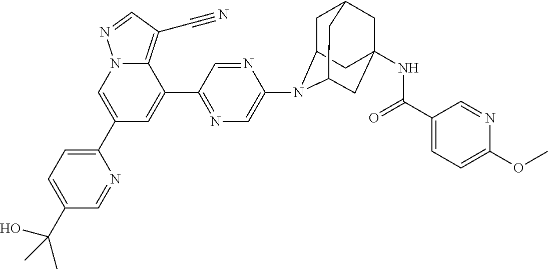 [SMILES: COc1ccc(C(=O)NC23CC4CC(C2)N(c2cnc(-c5cc(-c6ccc(C(C)(C)O)cn6)cn6ncc(C#N)c56)cn2)C(C4)C3)cn1] | N-((1R,3S,5s,7s)-2-(5-(3-cyano-6-(5-(2- hydroxypropan-2-yl)pyridin-2- yl)pyrazolo[1,5-a]pyridin-4-yl)pyrazin-2-yl)- 2-azaadamantan-5-yl)-6- methoxynicotinamide, |
| 3 | 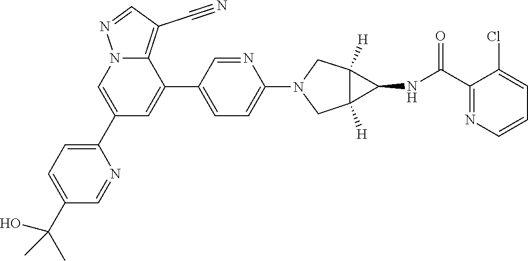 [SMILES: CC(C)(O)c1ccc(-c2cc(-c3ccc(N4C[C@@H]5[C@H](C4)[C@@H]5NC(=O)c4ncccc4Cl)nc3)c3c(C#N)cnn3c2)nc1] | 3-chloro-N-((1R,5S,6r)-3-(5-(3-cyano-6-(5- (2-hydroxypropan-2-yl)pyridin-2- yl)pyrazolo[1,5-a]pyridin-4-yl)pyridin-2-yl)- 3-azabicyclo[3.1.0]hexan-6-yl)picolinamide, |
| 4 | 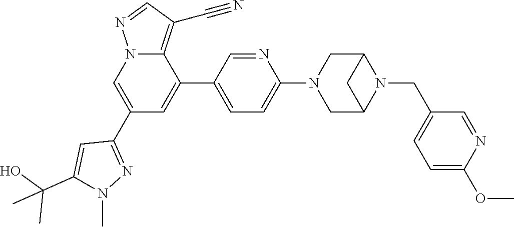 [SMILES: COc1ccc(CN2C3CC2CN(c2ccc(-c4cc(-c5cc(C(C)(C)O)n(C)n5)cn5ncc(C#N)c45)cn2)C3)cn1] | 6-(5-(2-hydroxypropan-2-yl)-1-methyl-1H- pyrazol-3-yl)-4-(6-(6-((6-methoxypyridin-3- yl)methyl)-3,6-diazabicyclo[3.1.1]heptan-3- yl)pyridin-3-yl)pyrazolo[1,5-a]pyridine-3- carbonitrile, |
| 5 | 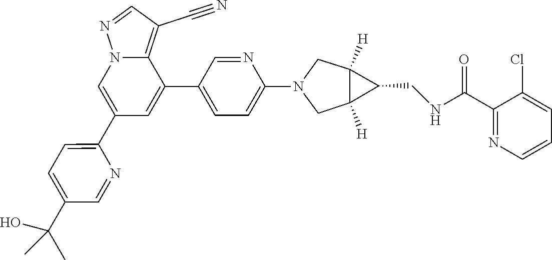 [SMILES: CC(C)(O)c1ccc(-c2cc(-c3ccc(N4C[C@@H]5[C@@H](CNC(=O)c6ncccc6Cl)[C@@H]5C4)nc3)c3c(C#N)cnn3c2)nc1] | 3-chloro-N-(((1R,5S,6s)-3-(5-(3-cyano-6-(5- (2-hydroxypropan-2-yl)pyridin-2- yl)pyrazolo[1,5-a]pyridin-4-yl)pyridin-2-yl)- 3-azabicyclo[3.1.0]hexan-6- yl)methyl)picolinamide, |
| 6 | 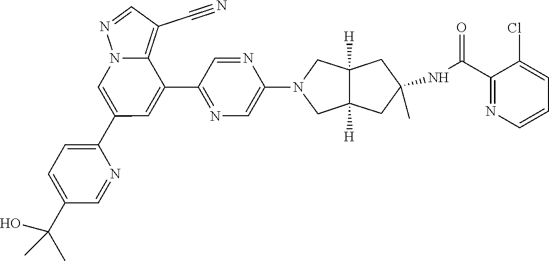 [SMILES: CC(C)(O)c1ccc(-c2cc(-c3cnc(N4C[C@@H]5C[C@@](C)(NC(=O)c6ncccc6Cl)C[C@@H]5C4)cn3)c3c(C#N)cnn3c2)nc1] | 3-chloro-N-((3aR,5s,6aS)-2-(5-(3-cyano-6-(5- (2-hydroxypropan-2-yl)pyridin-2- yl)pyrazolo[1,5-a]pyridin-4-yl)pyrazin-2-yl)- 5-methyloctahydrocyclopenta[c]pyrrol-5- yl)picolinamide, |
| 7 | 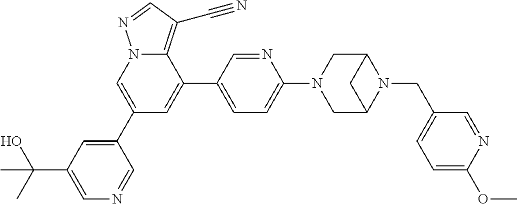 [SMILES: COc1ccc(CN2C3CC2CN(c2ccc(-c4cc(-c5cncc(C(C)(C)O)c5)cn5ncc(C#N)c45)cn2)C3)cn1] | 6-(5-(2-hydroxypropan-2-yl)pyridin-3-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 8 | 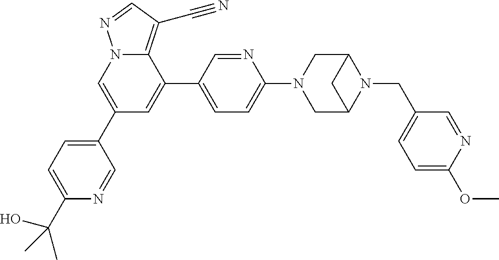 [SMILES: COc1ccc(CN2C3CC2CN(c2ccc(-c4cc(-c5ccc(C(C)(C)O)nc5)cn5ncc(C#N)c45)cn2)C3)cn1] | 6-(6-(2-hydroxypropan-2-yl)pyridin-3-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 9 | 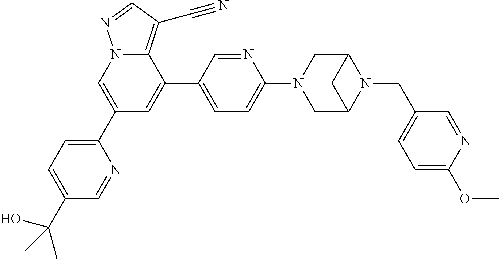 [SMILES: COc1ccc(CN2C3CC2CN(c2ccc(-c4cc(-c5ccc(C(C)(C)O)cn5)cn5ncc(C#N)c45)cn2)C3)cn1] | 6-(5-(2-hydroxypropan-2-yl)pyridin-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 10 | 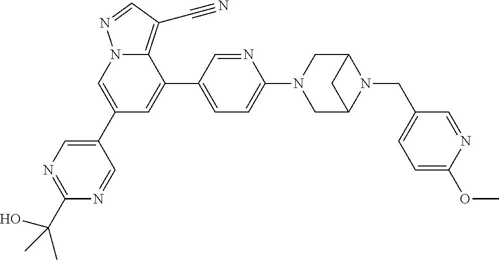 [SMILES: COc1ccc(CN2C3CC2CN(c2ccc(-c4cc(-c5cnc(C(C)(C)O)nc5)cn5ncc(C#N)c45)cn2)C3)cn1] | 6-(2-(2-hydroxypropan-2-yl)pyrimidin-5-yl)- 4-(6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 11 | 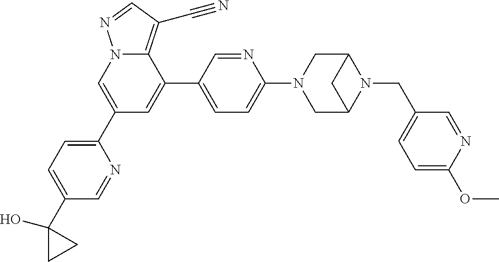 [SMILES: COc1ccc(CN2C3CC2CN(c2ccc(-c4cc(-c5ccc(C6(O)CC6)cn5)cn5ncc(C#N)c45)cn2)C3)cn1] | 6-(5-(1-hydroxycyclopropyl)pyridin-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 12 | 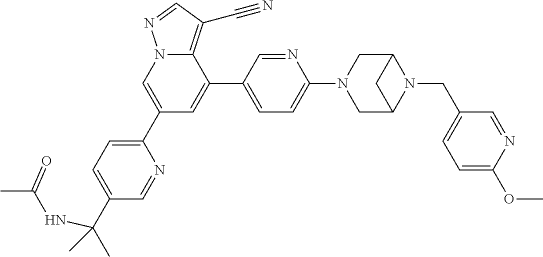 [SMILES: COc1ccc(CN2C3CC2CN(c2ccc(-c4cc(-c5ccc(C(C)(C)NC(C)=O)cn5)cn5ncc(C#N)c45)cn2)C3)cn1] | N-(2-(6-(3-cyano-4-(6-(6-((6- methoxypyridin-3-yl)methyl)-3,6- diazabicyclo[3.1.1]heptan-3-yl)pyridin-3- yl)pyrazolo[1,5-a]pyridin-6-yl)pyridin-3- yl)propan-2-yl)acetamide, |
| 13 | 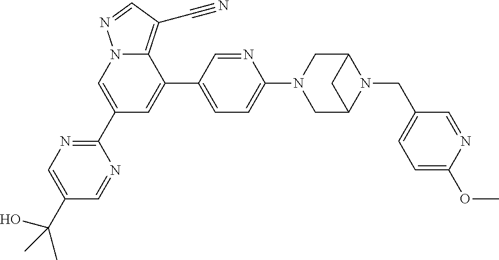 [SMILES: COc1ccc(CN2C3CC2CN(c2ccc(-c4cc(-c5ncc(C(C)(C)O)cn5)cn5ncc(C#N)c45)cn2)C3)cn1] | 6-(5-(2-hydroxypropan-2-yl)pyrimidin-2-yl)- 4-(6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 14 | 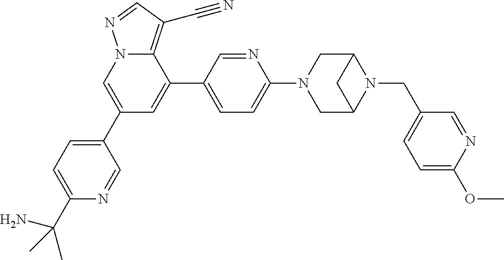 [SMILES: COc1ccc(CN2C3CC2CN(c2ccc(-c4cc(-c5ccc(C(C)(C)N)nc5)cn5ncc(C#N)c45)cn2)C3)cn1] | 6-(6-(2-aminopropan-2-yl)pyridin-3-yl)-4-(6- (6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 15 | 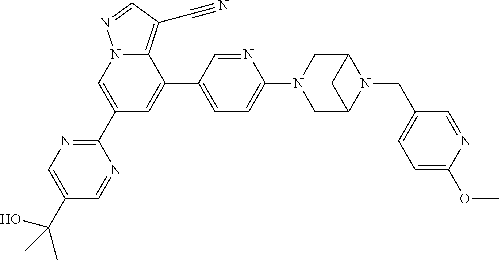 [SMILES: COc1ccc(CN2C3CC2CN(c2ccc(-c4cc(-c5ncc(C(C)(C)O)cn5)cn5ncc(C#N)c45)cn2)C3)cn1] | 6-(5-(2-hydroxypropan-2-yl)pyrazin-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 16 | 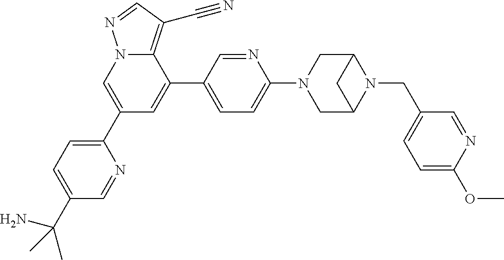 [SMILES: COc1ccc(CN2C3CC2CN(c2ccc(-c4cc(-c5ccc(C(C)(C)N)cn5)cn5ncc(C#N)c45)cn2)C3)cn1] | 6-(5-(2-aminopropan-2-yl)pyridin-2-yl)-4-(6- (6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 17 | 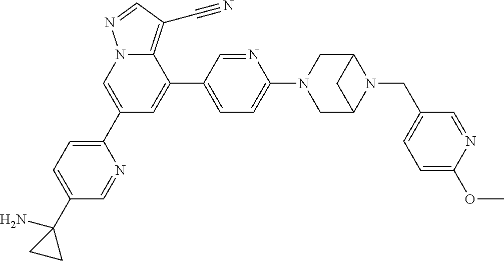 [SMILES: COc1ccc(CN2C3CC2CN(c2ccc(-c4cc(-c5ccc(C6(N)CC6)cn5)cn5ncc(C#N)c45)cn2)C3)cn1] | 6-(5-(1-aminocyclopropyl)pyridin-2-yl)-4-(6- (6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 18 | 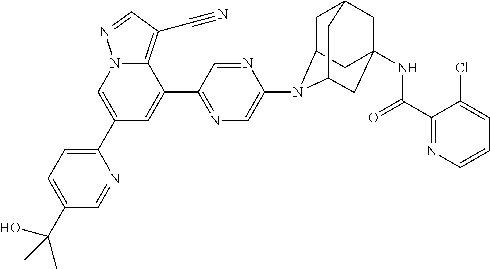 [SMILES: CC(C)(O)c1ccc(-c2cc(-c3cnc(N4C5CC6CC4CC(NC(=O)c4ncccc4Cl)(C6)C5)cn3)c3c(C#N)cnn3c2)nc1] | 3-chloro-N-((1R,3S,5s,7s)-2-(5-(3-cyano-6- (5-(2-hydroxypropan-2-yl)pyridin-2- yl)pyrazolo[1,5-a]pyridin-4-yl)pyrazin-2-yl)- 2-azaadamantan-5-yl)picolinamide, |
| 19 | 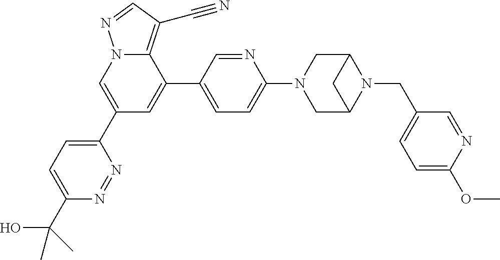 [SMILES: COc1ccc(CN2C3CC2CN(c2ccc(-c4cc(-c5ccc(C(C)(C)O)nn5)cn5ncc(C#N)c45)cn2)C3)cn1] | 6-(6-(2-hydroxypropan-2-yl)pyridazin-3-yl)- 4-(6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 20 | 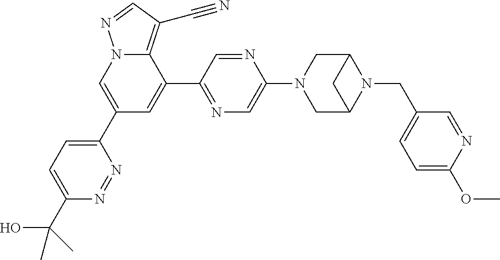 [SMILES: COc1ccc(CN2C3CC2CN(c2cnc(-c4cc(-c5ccc(C(C)(C)O)nn5)cn5ncc(C#N)c45)cn2)C3)cn1] | 6-(6-(2-hydroxypropan-2-yl)pyridazin-3-yl)- 4-(5-(6-((6-methoxypyridin-3-yl)methyl)-3,6- diazabicyclo[3.1.1]heptan-3-yl)pyrazin-2- yl)pyrazolo[1,5-a]pyridine-3-carbonitrile, |
| 21 | 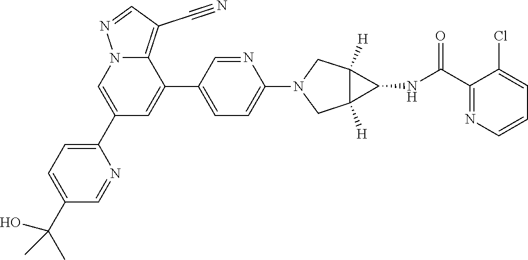 [SMILES: CC(C)(O)c1ccc(-c2cc(-c3ccc(N4C[C@@H]5[C@H](C4)[C@H]5NC(=O)c4ncccc4Cl)nc3)c3c(C#N)cnn3c2)nc1] | 3-chloro-N-((1R,5S,6s)-3-(5-(3-cyano-6-(5- (2-hydroxypropan-2-yl)pyridin-2- yl)pyrazolo[1,5-a]pyridin-4-yl)pyridin-2-yl)- 3-azabicyclo[3.1.0]hexan-6-yl)picolinamide, |
| 22 | 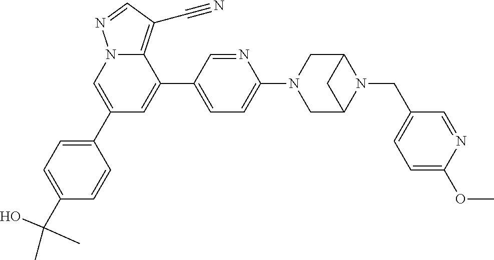 [SMILES: COc1ccc(CN2C3CC2CN(c2ccc(-c4cc(-c5ccc(C(C)(C)O)cc5)cn5ncc(C#N)c45)cn2)C3)cn1] | 6-(4-(2-hydroxypropan-2-yl)phenyl)-4-(6-(6- ((6-methoxypyridin-3-yl)methyl)-3,6- diazabicyclo[3.1.1]heptan-3-yl)pyridin-3- yl)pyrazolo[1,5-a]pyridine-3-carbonitrile, |
| 23 | 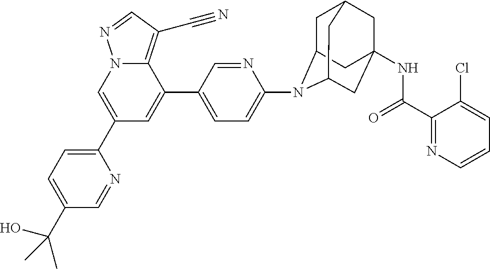 [SMILES: CC(C)(O)c1ccc(-c2cc(-c3ccc(N4C5CC6CC4CC(NC(=O)c4ncccc4Cl)(C6)C5)nc3)c3c(C#N)cnn3c2)nc1] | 3-chloro-N-((1R,3S,5s,7s)-2-(5-(3-cyano-6- (5-(2-hydroxypropan-2-yl)pyridin-2- yl)pyrazolo[1,5-a]pyridin-4-yl)pyridin-2-yl)- 2-azaadamantan-5-yl)picolinamide, |
| 24 | 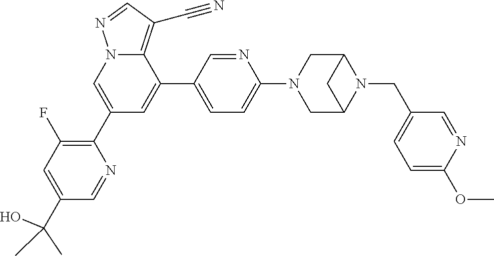 [SMILES: COc1ccc(CN2C3CC2CN(c2ccc(-c4cc(-c5ncc(C(C)(C)O)cc5F)cn5ncc(C#N)c45)cn2)C3)cn1] | 6-(3-fluoro-5-(2-hydroxypropan-2-yl)pyridin- 2-yl)-4-(6-(6-((6-methoxypyridin-3- yl)methyl)-3,6-diazabicyclo[3.1.1]heptan-3- yl)pyridin-3-yl)pyrazolo[1,5-a]pyridine-3- carbonitrile, |
| 25 | 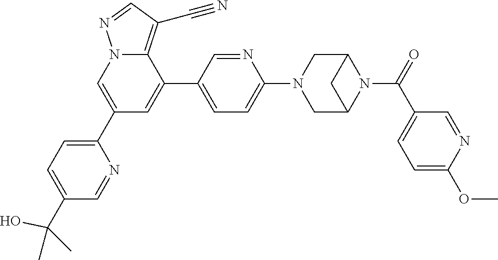 [SMILES: COc1ccc(C(=O)N2C3CC2CN(c2ccc(-c4cc(-c5ccc(C(C)(C)O)cn5)cn5ncc(C#N)c45)cn2)C3)cn1] | 6-(5-(2-hydroxypropan-2-yl)pyridin-2-yl)-4- (6-(6-(6-methoxynicotinoyl)-3,6- diazabicyclo[3.1.1]heptan-3-yl)pyridin-3- yl)pyrazolo[1,5-a]pyridine-3-carbonitrile, |
| 26 | 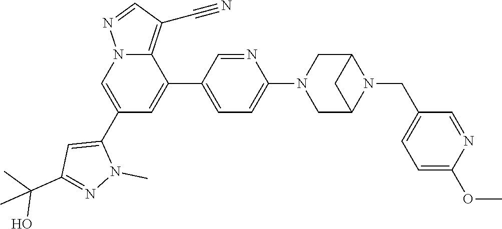 [SMILES: COc1ccc(CN2C3CC2CN(c2ccc(-c4cc(-c5cc(C(C)(C)O)nn5C)cn5ncc(C#N)c45)cn2)C3)cn1] | 6-(3-(2-hydroxypropan-2-yl)-1-methyl-1H- pyrazol-5-yl)-4-(6-(6-((6-methoxypyridin-3- yl)methyl)-3,6-diazabicyclo[3.1.1]heptan-3- yl)pyridin-3-yl)pyrazolo[1,5-a]pyridine-3- carbonitrile, |
| 27 | 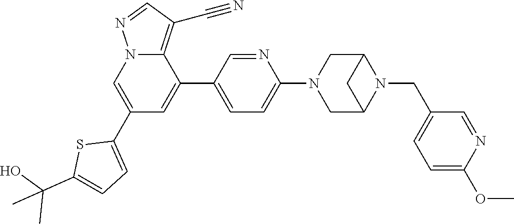 [SMILES: COc1ccc(CN2C3CC2CN(c2ccc(-c4cc(-c5ccc(C(C)(C)O)s5)cn5ncc(C#N)c45)cn2)C3)cn1] | 6-(5-(2-hydroxypropan-2-yl)thiophen-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 28 | 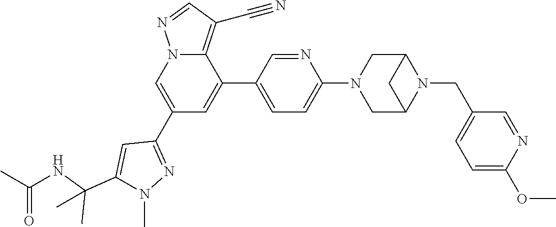 [SMILES: COc1ccc(CN2C3CC2CN(c2ccc(-c4cc(-c5cc(C(C)(C)NC(C)=O)n(C)n5)cn5ncc(C#N)c45)cn2)C3)cn1] | N-(2-(3-(3-cyano-4-(6-(6-((6- methoxypyridin-3-yl)methyl)-3,6- diazabicyclo[3.1.1]heptan-3-yl)pyridin-3- yl)pyrazolo[1,5-a]pyridin-6-yl)-1-methyl-1H- pyrazol-5-yl)propan-2-yl)acetamide, |
| 29 | 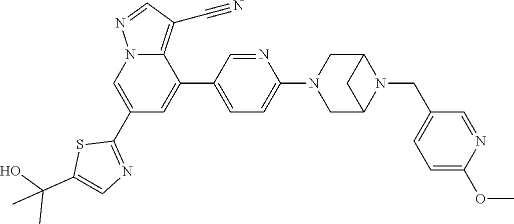 [SMILES: COc1ccc(CN2C3CC2CN(c2ccc(-c4cc(-c5ncc(C(C)(C)O)s5)cn5ncc(C#N)c45)cn2)C3)cn1] | 6-(5-(2-hydroxypropan-2-yl)thiazol-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 30 | 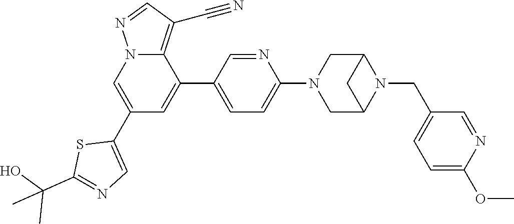 [SMILES: COc1ccc(CN2C3CC2CN(c2ccc(-c4cc(-c5cnc(C(C)(C)O)s5)cn5ncc(C#N)c45)cn2)C3)cn1] | 6-(2-(2-hydroxypropan-2-yl)thiazol-5-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 31 | 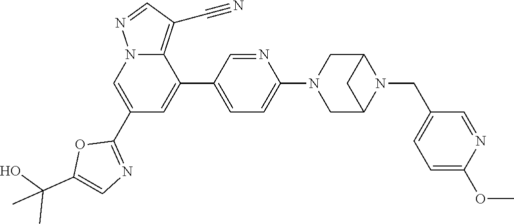 [SMILES: COc1ccc(CN2C3CC2CN(c2ccc(-c4cc(-c5ncc(C(C)(C)O)o5)cn5ncc(C#N)c45)cn2)C3)cn1] | 6-(5-(2-hydroxypropan-2-yl)oxazol-2-yl)-4- (6-(6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 32 | 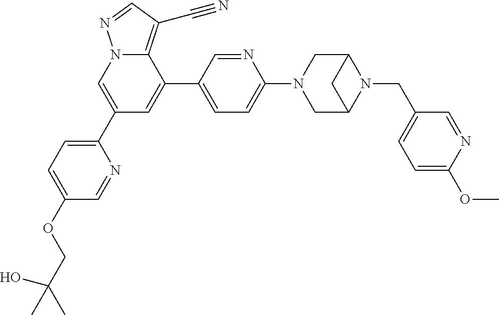 [SMILES: COc1ccc(CN2C3CC2CN(c2ccc(-c4cc(-c5ccc(OCC(C)(C)O)cn5)cn5ncc(C#N)c45)cn2)C3)cn1] | 6-(5-(2-hydroxy-2-methylpropoxy)pyridin-2- yl)-4-(6-(6-((6-methoxypyridin-3-yl)methyl)- 3,6-diazabicyclo[3.1.1]heptan-3-yl)pyridin-3- yl)pyrazolo[1,5-a]pyridine-3-carbonitrile, |
| 33 | 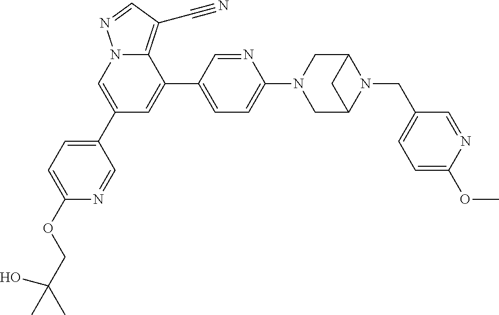 [SMILES: COc1ccc(CN2C3CC2CN(c2ccc(-c4cc(-c5ccc(OCC(C)(C)O)nc5)cn5ncc(C#N)c45)cn2)C3)cn1] | 6-(6-(2-hydroxy-2-methylpropoxy)pyridin-3- yl)-4-(6-(6-((6-methoxypyridin-3-yl)methyl)- 3,6-diazabicyclo[3.1.1]heptan-3-yl)pyridin-3- yl)pyrazolo[1,5-a]pyridine-3-carbonitrile, |
| 34 | 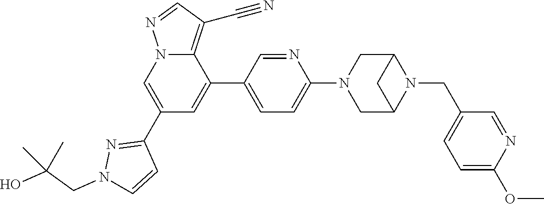 [SMILES: COc1ccc(CN2C3CC2CN(c2ccc(-c4cc(-c5ccn(CC(C)(C)O)n5)cn5ncc(C#N)c45)cn2)C3)cn1] | 6-(1-(2-hydroxy-2-methylpropyl)-1H- pyrazol-3-yl)-4-(6-(6-((6-methoxypyridin-3- yl)methyl)-3,6-diazabicyclo[3.1.1]heptan-3- yl)pyridin-3-yl)pyrazolo[1,5-a]pyridine-3- carbonitrile, |
| 35 | 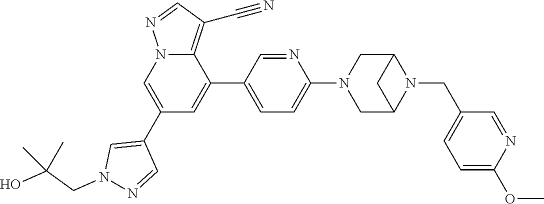 [SMILES: COc1ccc(CN2C3CC2CN(c2ccc(-c4cc(-c5cnn(CC(C)(C)O)c5)cn5ncc(C#N)c45)cn2)C3)cn1] | 6-(1-(2-hydroxy-2-methylpropyl)-1H- pyrazol-4-yl)-4-(6-(6-((6-methoxypyridin-3- yl)methyl)-3,6-diazabicyclo[3.1.1]heptan-3- yl)pyridin-3-yl)pyrazolo[1,5-a]pyridine-3- carbonitrile, |
| 36 | 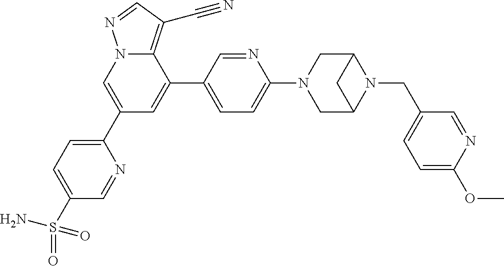 [SMILES: COc1ccc(CN2C3CC2CN(c2ccc(-c4cc(-c5ccc(S(N)(=O)=O)cn5)cn5ncc(C#N)c45)cn2)C3)cn1] | 6-(3-cyano-4-(6-(6-((6-methoxypyridin-3- yl)methyl)-3,6-diazabicyclo[3.1.1]heptan-3- yl)pyridin-3-yl)pyrazolo[1,5-a]pyridin-6- yl)pyridine-3-sulfonamide, and |
| 37 | 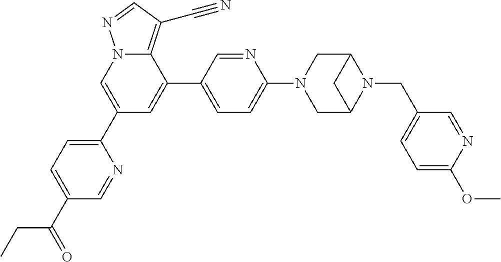 [SMILES: CCC(=O)c1ccc(-c2cc(-c3ccc(N4CC5CC(C4)N5Cc4ccc(OC)nc4)nc3)c3c(C#N)cnn3c2)nc1] | 4-(6-(6-((6-methoxypyridin-3-yl)methyl)-3,6- diazabicyclo[3.1.1]heptan-3-yl)pyridin-3-yl)- 6-(5-propionylpyridin-2-yl)pyrazolo[1,5- a]pyridine-3-carbonitrile. |

Embodiment 43. A pharmaceutical composition comprising a compound of any one of Embodiments 1-42, and/or a stereoisomer, a stable isotope, or a pharmaceutically acceptable salt or solvate thereof, admixed with at least one pharmaceutically acceptable carrier.

Embodiment 44. The pharmaceutical composition of Embodiment 43, further comprising at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, another RET kinase inhibitor, and kinase inhibitors.

Embodiment 45. The pharmaceutical composition of Embodiment 44, wherein the at least one therapeutic co-agent or co-treatment is combined with the compound in a single dosage form, or the at least one therapeutic co-agent is administered simultaneously or sequentially as separate dosage forms.

Embodiment 46. A method to treat a RET-associated disease in a patient in need thereof, comprising administering to the subject in need of such treatment a therapeutically effective amount of a compound of any one of Embodiments 1-42, and/or a stereoisomer, a stable isotope, or a pharmaceutically acceptable salt or solvate thereof, or a pharmaceutical composition of any one of Embodiments 43-45.

Embodiment 47. The method of Embodiment 46, wherein the method comprises determining if the disease in the patient is a RET-associated disease, and administering to a subject in need of such treatment a therapeutically effective amount of a compound of any one of Embodiments 1-42, and/or a stereoisomer, a stable isotope, or a pharmaceutically acceptable salt or solvate thereof, or a pharmaceutical composition of any one of Embodiments 43-45.

Embodiment 48. The method of Embodiment 46 or Embodiment 47, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 49. The method of Embodiment 46 or Embodiment 47, wherein the RET-associated disease is irritable bowel syndrome or other gastrointestinal disorder having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 50. The method of Embodiment 48, wherein the treatment comprises administering at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

Embodiment 51. The method of Embodiment 50, wherein the administering the compound is conducted simultaneously or serially with the administering of the therapeutic co-agent.

Embodiment 52. The method of Embodiment 51, wherein the administering the therapeutic co-agent comprises another RET inhibitor, an immunotherapy, or combination thereof.

Embodiment 53. The method of Embodiment 48, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 54. The method of any of one of Embodiments 46-53, wherein the compound of any one of Embodiments 1-42, and/or a stereoisomer, a stable isotope, or a pharmaceutically acceptable salt or solvate thereof, or a pharmaceutical composition of any one of Embodiments 43-45, is orally administered.

Embodiment 55. A use of a compound of any one of Embodiments 1-42, and/or a stereoisomer, a stable isotope, or a pharmaceutically acceptable salt or solvate thereof, or a pharmaceutical composition according to any one of Embodiments 42-45, as a medicament, in the manufacture of a medicament, or in medicine for treatment of a RET-associated disease.

Embodiment 56. The use of Embodiment 55, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 57. The use of Embodiment 56, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 58. The use of Embodiment 56 or Embodiment 57, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 59. The use of any of one of Embodiments 55-58, wherein the medicament is formulated for oral administration.

Embodiment 60. A compound of any one of Embodiments 1-42, and/or a stereoisomer, a stable isotope, or a pharmaceutically acceptable salt or solvate thereof, or a pharmaceutical composition of Embodiments 43-45, for use in treating a RET-associated disease.

Embodiment 61. The compound or pharmaceutical composition for use of Embodiment 60, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 62. The compound or pharmaceutical composition for use of Embodiment 60, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

Embodiment 63. The compound or pharmaceutical composition for use of Embodiment 60 or Embodiment 61, wherein the RET-associated disease is a RET-associated cancer, and the use comprises determining if the cancer in a patient is RET-associated cancer, and administering to the patient in need of such treatment a therapeutically effective amount of the compound or pharmaceutical composition.

Embodiment 64. The compound or pharmaceutical composition for use of Embodiment 61 or Embodiment 63, wherein the RET-associated cancer is selected from lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, ovarian cancer, and myeloproliferative cancer.

Embodiment 65. A method of inhibiting RET kinase activity in vitro or in vivo for a RET-associated cancer cell having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein, with a compound of any one of Embodiments 1-42, and/or a stereoisomer, a stable isotope, or a pharmaceutically acceptable salt or solvate thereof.

Embodiment 66. A method of treating RET-associated cancer in a patient who has developed resistance to a RET inhibitor, comprising administering to a subject in need of such treatment a therapeutically effective amount of a compound of any one of Embodiments 1-42, and/or a stereoisomer, a stable isotope, or a pharmaceutically acceptable salt or solvate thereof, or a pharmaceutical composition of any one of Embodiments 43-45.

Embodiment 67. The method of Embodiment 51, wherein the method comprises (a) determining the RET-mutations of a cancer cell in a sample from a patient who developed resistance to prior treatment of a RET inhibitor; and (b) administering a compound of any one of Embodiments 1-42, and/or a stereoisomer, a stable isotope, or a pharmaceutically acceptable salt or solvate thereof, or a pharmaceutical composition of any one of Embodiments 43-45.

Embodiment 68. The method of Embodiment 66 or Embodiment 67, wherein the treatment comprises administering at least one therapeutic co-agent or co-treatment selected from chemotherapeutics or other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

Embodiment 69. The method of Embodiment 68, wherein administering the therapeutic co-agent comprises another RET inhibitor, an immunotherapy, or combination thereof.

Embodiment 70. A kit comprising a compound of any of Embodiments 1-42 or a pharmaceutically acceptable salt thereof, or a pharmaceutical composition according to any of Embodiments 43-45, and a therapeutic co-agent.

In some embodiments, the compound of Formula I (such as a compound selected from the compounds of Formulae IA, IB, IC, ID, IE, IIA, IIB, IIC, IID, and IIE), has the chiral configuration shown in excess over its enantiomer, so the compound is optically active. For example, such compounds disclosed herein are substantially free of the opposite enantiomer, i.e., at least 95% of the compound has the chirality shown above.

Also disclosed herein is a pharmaceutical composition comprising a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solate thereof, and a pharmaceutically acceptable carrier.

Further disclosed herein is a method of inhibiting the activity of RET comprising contacting the protein RET with an effective amount of a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof disclosed herein.

Further disclosed herein is a method of treating a disease treatable by inhibition of RET in a patient, comprising administering to the patient in recognized need of such treatment, an effective amount of a pharmaceutical composition comprising a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof disclosed herein and a pharmaceutically acceptable carrier.

Further disclosed herein is a method of treating a cancer in a patient, comprising administering to the patient in recognized need of such treatment, an effective amount of a pharmaceutical composition comprising a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof disclosed herein and a pharmaceutically acceptable carrier. In some embodiments, the cancer is colon cancer, gastric cancer, leukemia, lymphoma, melanoma, or pancreatic cancer.

Further disclosed herein is a method of treating an inflammatory disease in a patient, comprising administering to the patient in recognized need of such treatment, an effective amount of a pharmaceutical composition comprising a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof disclosed herein and a pharmaceutically acceptable carrier. In some embodiments, the inflammatory disease is rheumatoid arthritis, psoriasis, or eczema.

Further disclosed herein is a use of a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof in preparation of a medication for treating a disease responsive to inhibition of RET, such as a cancer. In some embodiments, the cancer is lung cancers, thyroid cancers, pancreatic cancers, salivary gland cancers, spitz tumors, colorectal cancers, ovarian cancers, or myeloproliferative cancers.

The pharmaceutical composition comprising a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof, and a pharmaceutically acceptable carrier, can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be administered orally in solid dosage forms, such as capsules, tablets, troches, dragées, granules and powders, or in liquid dosage forms, such as elixirs, syrups, emulsions, dispersions, and suspensions. The compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can also be administered parenterally, in sterile liquid dosage forms, such as dispersions, suspensions or solutions. Other dosages forms that can also be used to administer the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof include ointment, cream, drops, transdermal patch or powder for topical administration, an ophthalmic solution or suspension formation, i.e., eye drops, for ocular administration, an aerosol spray or powder composition for inhalation or intranasal administration, or a cream, ointment, spray or suppository for rectal or vaginal administration.

Gelatin capsules containing the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof and at least one powdered carrier selected, for example, from lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound disclosed herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the pharmaceutical composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are disclosed in Remington's Pharmaceutical Sciences, A. Osol, a standard reference text in the art.

The compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be examined for efficacy in treating cancer by in vivo assays. For example, the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be administered to an animal (e.g., a mouse model) having cancer and its therapeutic effects can be accessed. Positive results in one or more of such tests are sufficient to increase the scientific storehouse of knowledge and hence sufficient to demonstrate practical utility of the compounds and/or salts tested. Based on the results, an appropriate dosage range and administration route for animals, such as humans, can also be determined.

For administration by inhalation, the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof may be conveniently delivered in the form of an aerosol spray presentation from pressurized packs or nebulisers. The compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof may also be delivered as powders, which may be formulated and the powder composition may be inhaled with the aid of an insufflation powder inhaler device. One exemplary delivery system for inhalation can be a metered dose inhalation (MDI) aerosol, which may be formulated as a suspension or solution of a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof in at least one suitable propellant, selected, for example, from fluorocarbons and hydrocarbons.

For ocular administration, an ophthalmic preparation may be formulated with an appropriate weight percentage of a solution or suspension of the compound of Formula I (such as a compound selected from the compounds of Formulae IA, IB, IC, ID, IE, IIA, IIB, IIC, IID, and IIE) and/or a stereoisomer, a stable isotope, or a pharmaceutically acceptable salt or solvate thereof in an appropriate ophthalmic vehicle, such that the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof is maintained in contact with the ocular surface for a sufficient time period to allow the compound to penetrate the corneal and internal regions of the eye.

Useful pharmaceutical dosage-forms for administration of the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be present in an amount of 1, 5, 10, 15, 20, 25, 50, 75, 80, 85, 90, 95, 100, 125, 150, 200, 250, 300, 400 and 500 mg in a capsule.

In some embodiments, a large number of unit capsules can be prepared by filling standard two-piece hard gelatin capsules each with, for example, 100 milligrams of the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof in powder, 150 milligrams of lactose, 50 milligrams of cellulose, and 6 milligrams magnesium stearate.

In some embodiments, a mixture of the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof and a digestible oil such as soybean oil, cottonseed oil or olive oil can be prepared and injected by means of a positive displacement pump into gelatin to form soft gelatin capsules containing 75 or 100 milligrams of the active ingredient. The capsules are washed and dried.

In some embodiments, the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be present in an amount of 1, 5, 10, 15, 20, 25, 50, 75, 80, 85, 90, 95, 100, 125, 150, 200, 250, 300, 400 and 500 mg in a tablet.

In some embodiments, a large number of tablets can be prepared by conventional procedures so that the dosage unit comprises, for example, 100 milligrams of the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may, for example, be applied to increase palatability or delay absorption.

In some embodiments, a parenteral composition suitable for administration by injection can be prepared by stirring 1.5% by weight of a compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof are administered stepwise or in conjunction with at least one other therapeutic agent. When drugs are administered in physical combination, the dosage form and administration route should be selected depending on the compatibility of the combined drugs. Thus, the term “co-administration” is understood to include the administration of at least two agents concomitantly or sequentially, or alternatively as a fixed dose combination of the at least two active components.

The compound of Formula I (such as a compound selected from the compounds of Formulae IA, IB, IC, ID, IE, IIA, IIB, IIC, IID, and IIE), and/or a stereoisomer, a stable isotope, or a pharmaceutically acceptable salt or solvate thereof can be administered as the sole active ingredient or in combination with at least one second active ingredient, selected, for example, from other active ingredients known to be useful for treating the target disease, such as cancers including, for example, colon cancer, gastric cancer, leukemia, lymphoma, melanoma, and pancreate cancer in a patient.

As used herein, the term “optical isomer” or “stereoisomer” refers to any of the various stereo isomeric configurations which may exist for a given compound of the present disclosure and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. The present disclosure includes enantiomers, diastereomers or racemates of the compounds. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog IR-SJ system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and synthesis procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present disclosure includes all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration unless specified. If the compound contains a di-substituted cycloalkyl, the cycloalkyl substituent may have a cis- or trans-configuration, unless otherwise specified.

In many cases, the compounds of the present disclosure are capable of forming acid and/or base salts by virtue of the presence of amino and/or carboxyl groups or groups similar thereto. As used herein, the terms “salt” or “salts” refers to an acid addition or base addition salt of a compound of the disclosure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable.

Pharmaceutically acceptable acid addition salts can be formed with inorganic acids and organic acids, e.g., acetate, adipate, aluminum, ascorbate, aspartate, benzoate, besylate, bromide/hydrobromide, bicarbonate/carbonate, bisulfate/sulfate, camphorsulfonate, caproate, chloride/hydrochloride, chloroprocaine, chlortheophyllonate, citrate, edetate, calcium edetate, ethandisulfonate, ethylsulfonate, ethylene diamine, fumarate, galactarate (mucate), gluceptate, gluconate, glucuronate, glutamate, glycolate, hexyl resorcinate, hippurate, hydroiodide/iodide, hydroxynapthoate (xinafoate), isethionate, lactate, lactobionate, laurylsulfate, lithium, malate, maleate, malonate, mandelate, mesylate, methylsulphate, naphthoate, napsylate, nicotinate, nitrate, octadecanoate, oleate, oxalate, palmitate, pamoate, pantothenate, phosphate/hydrogen phosphate/dihydrogen phosphate, polygalacturonate, procaine, propionate, salicylate, sebacate, stearate, subacetate, succinate, sulfate, sulfosalicylate, tannate, tartrate, bitartrate, tosylate, triphenylacetate, and trifluoroacetate salts. Lists of additional suitable salts can be found, e.g., in REMINGTON'S PHARMACEUTICAL SCIENCES, 20th ed., Mack Publishing Company, Easton, Pa., (1985); and in HANDBOOK OF PHARMACEUTICAL SALTS: PROPERTIES, SELECTION, AND USE, by Stahl and Wermuth (Wiley-VCH, Weinheim, Germany, 2002). Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic, sulfosalicylic acid, and the like.

Pharmaceutically acceptable base addition salts can be formed with inorganic or organic bases and can have inorganic or organic counterions.

Inorganic counterions for such base salts include, for example, ammonium salts and metals from columns I to XII of the periodic table. In certain embodiments, the counterion is selected from sodium, potassium, ammonium, alkylammonium having one to four C1-C4 alkyl groups, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Suitable organic amines include isopropylamine, benzathine, cholinate, diethanolamine, diethylamine, lysine, meglumine, piperazine and tromethamine.

The pharmaceutically acceptable salts of the present disclosure can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, tetrahydrofuran, toluene, chloroform, dichloromethane, methanol, ethanol, isopropanol, or acetonitrile is desirable, where practicable.

Any formula given herein is intended to represent unlabeled forms (i.e., compounds wherein all atoms are present at natural isotopic abundances and not isotopically enriched) as well as isotopically enriched or labeled forms of the compounds. Isotopically enriched or labeled compounds have structures depicted by the formulas given herein except that at least one atom of the compound is replaced by an atom of the same element but having an atomic mass or mass number different from the atomic mass or the atomic mass distribution that occurs naturally. Examples of isotopes that can be incorporated into enriched or labeled compounds of the disclosure include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, and ¹²⁵I respectively. The present disclosure includes various isotopically labeled compounds as defined herein, for example those in which radioactive isotopes, such as ³H and ¹⁴C, or those in which non-radioactive isotopes, such as ²H and ¹³C, are present at levels significantly above the natural abundance for these isotopes. These isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of Formula I can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the Formula I if it is incorporated at substantially above the level of natural isotopic abundance. The present disclosure includes isotopically enriched versions of the compounds, e.g., deuterated versions as well as non-deuterated versions. Deuterated versions may be deuterated at a single site, or at multiple sites.

The degree of incorporation of such an isotope in an isotopically-enriched compound, particularly deuterium, may be defined by the isotopic enrichment factor. The term “isotopic enrichment factor” as used herein means the ratio between the isotopic abundance of a specified isotope in a sample, and the natural abundance of the isotope in a non-enriched sample. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the present disclosure include those wherein the solvent of crystallization may be isotopically substituted, e.g., D₂O, d₆-acetone, d₆-DMSO, as well as solvates with non-enriched solvents.

Compounds of the disclosure, e.g., compounds of Formula I (such as a compound selected from the compounds of Formulae IA, IB, IC, ID, IE, IIA, IIB, IIC, IID, and IIE), that contain groups capable of acting as donors and/or acceptors for hydrogen bonds, may be capable of forming co-crystals with suitable co-crystal formers. These co-crystals may be prepared from compounds of Formula I (such as a compound selected from the compounds of Formulae IA, IB, IC, ID, IE, IIA, IIB, IIC, IID, and IIE), by known co-crystal forming procedures. Such procedures include grinding, heating, co-subliming, co-melting, or contacting in solution compounds of Formula I (such as a compound selected from the compounds of Formulae IA, IB, IC, ID, IE, IIA, IIB, IIC, IID, and IIE), with the co-crystal former under crystallization conditions and isolating co-crystals thereby formed. Suitable co-crystal formers include those described in WO2004078163. Hence the present disclosure further provides co-crystals comprising a compound of Formula I (such as a compound selected from the compounds of Formulae IA, IB, IC, ID, IE, IIA, IIB, IIC, IID, and IIE).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, REMINGTON'S PHARMACEUTICAL SCIENCES, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “therapeutically effective amount” refers to the amount of the compound of the present disclosure that, when administered to a subject, is effective to (1) at least partially alleviate, inhibit, prevent and/or ameliorate a condition, or a disorder or a disease (i) mediated by a kinase such as RET or (ii) associated with activity of a kinase such as RET, or (iii) characterized by activity (normal or abnormal) of RET; or (2) reduce or inhibit the activity of RET or (3) reduce or inhibit the expression of RET.

In another non-limiting embodiment, the term “a therapeutically effective amount” refers to the amount of the compound of the present disclosure that, when administered to a cell, or a tissue, or a non-cellular biological material, or a medium, is effective to at least partially reduce or inhibit the activity of RET, or at least partially reduce or inhibit the expression of RET.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or inhibiting” refers to the reduction or suppression of a given condition, activity, effect, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “Treat”, “treating” or “treatment” refers to alleviating or ameliorating at least one physical parameter including those which may not be discernible by the patient. In yet another embodiment, “Treat”, “treating” or “treatment” refers to modulating the disease or disorder, either physically, (e.g., stabilization of a discernible symptom), physiologically, (e.g., stabilization of a physical parameter), or both. In yet another embodiment, “Treat”, “treating” or “treatment” refers to delaying the development or progression of the disease or disorder.

As used herein, a subject is “in need of” a treatment if such subject would be expected to benefit biologically, medically or in quality of life from such treatment.

As used herein, the term “a” “an” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosed otherwise claimed.

Any asymmetric atom (e.g., carbon or the like) of the compound(s) of the present disclosure can be present in racemic or enantiomerically enriched, for example, the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the (R)- or (S)-configuration; i.e., for optically active compounds, it is often, for example, to use one enantiomer to the substantial exclusion of the other enantiomer. Substituents at atoms with carbon-carbon double bonds may, where possible, be present in cis-(Z)- or trans-(E)-form, and both are included in the present disclosure unless otherwise indicated.

Accordingly, as used herein a compound of the present disclosure can be in the form of one of the possible isomers, rotamers, atropisomers, or as a mixture thereof, for example, as substantially pure geometric (cis or trans) isomers, diastereomers, optical isomers (antipodes), racemates or mixtures thereof. ‘Substantially pure” or “substantially free of other isomers” as used herein means the product contains less than 5%, and, such as, less than 2%, of other isomers relative to the amount of the preferred isomer, by weight.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present disclosure, including their salts, can also be obtained in the form of their hydrates, or include other solvents used for their crystallization. The compounds of the present disclosure may inherently or by design form solvates with pharmaceutically acceptable solvents (including water); therefore, it is intended that the present disclosure embraces both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Schemes 1-2 show general methods for preparing the compounds of the present disclosure as well as intermediates. The detailed description and syntheses are disclosed in the Examples below. Those skilled in the art will be able to find other synthetic methods or modify the methods described below using conventional chemistry for preparing suitable compounds encompassed by Formula I. So these methods are equally applicable to preparation of compounds with other embodiments. Although specific starting materials and reagents are depicted in the Schemes and discussed below, other starting materials and reagents can be easily substituted to provide a variety of compounds and/or reaction conditions.

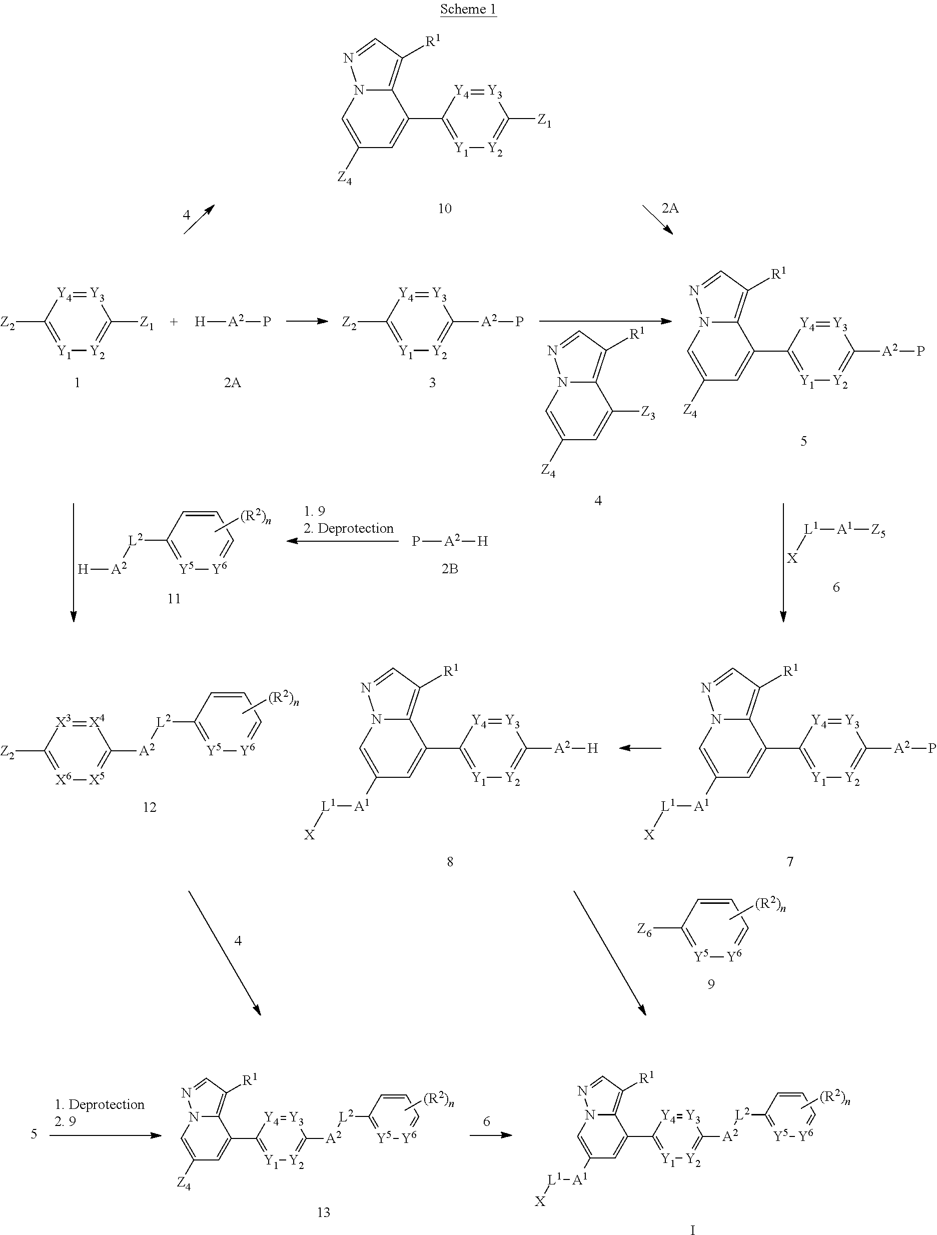

Compounds of Formula I can be made by general synthetic methods as illustrated in Scheme 1, wherein R¹, R², A¹-A², X, Y¹-Y⁶, and n are the same as those defined in Embodiment 1 above. Compounds 1, 2A, 2B, 4, 6 and 9 can be made by many methods known to the skilled person or are commercially available. Compound 1 (Z₁ and Z₂ are independently Cl, Br, I, or OTf) can react with compound 2A (P is a protecting group such as Boc, Cbz or benzyl) under Buchwald reaction conditions of palladium chemistry or nucleophilic displacement of Z₁ of compound 1 by compound 2A to give compound 3. The reactive selectivity between Z₁ and Z₂ can be controlled by placing different groups at Z₁ and Z₂, or Z₁ and Z₂ can be the same when the compound 1 is symmetrical. For example, one can start with compound 1 wherein Z₁ is Br and Z₂ is Cl or F. Compound 3 can be converted to the boronic acid or pinacol boron ester with bis(pinacolato)diboron using palladium catalyzed chemistry, which then undergoes Suzuki reaction with pyrazolo[1,5-a]pyridine 4 (Z₃ and Z₄ are independently Cl, Br, I, or OTf) using palladium catalyzed chemistry to give compound 5. The reactive selectivity between Z₃ and Z₄ can be controlled by placing different groups at Z₃ and Z₄. For example, one can start with compound 4 wherein Z₃ is OTf and Z₄ is Br. Another method is to have Z₃ be halogen and Z₄ be OP (P is a protecting group); the latter can be deprotected and converted to triflate in the next reaction. Suzuki coupling of compound 5 with compound 6 (Z₅ is boronic acid or pinacol boron ester) can be carried out to give compound 7. Alternatively, compound 5 can be converted to the boronic acid or pinacol boron ester with bis(pinacolato)diboron using palladium catalyzed chemistry, which then undergoes Suzuki reaction with compound 6 (Z₅ is Cl, Br, I, or OTf) using palladium catalyzed chemistry to give compound 7. Compound 7 is deprotected under appropriate conditions depending on the type of protective groups to give compound 8, such as hydrochloric acid or TFA for Boc, hydrogenolysis for benzyl or CBZ. Coupling reaction of compound 8 with compound 9 (Z₆ is —W—CHO, —CO₂H, —COCl, or —SO₂Cl, wherein W is C1-C5 alkylenyl, or C1-C5 haloalkylenyl) can be carried out under appropriate reaction conditions to give compounds of Formula I. For example, the coupling condition is reductive amination for the compound 8 in which Z₆ is the aldehyde group or under basic condition for the compound 8 in which Z₆ is —COCl or —SO₂Cl. Alternatively, compound 5 can be made by reaction of compound 1 with compound 4 to give compound 10 under similar Suzuki reaction conditions and then reaction of compound 10 with compound 2A under Buchwald, or nucleophilic displacement reaction conditions as described above.

There are other alternative methods that can be used to make compounds of Formula I. Reaction of compound 1 with compound 11 under Buchwald reaction or nucleophilic displacement reaction conditions as described above gives compound 12. Compound 11 can be made from reaction of compound 2B and compound 9 under Buchwald reaction conditions of palladium chemistry or nucleophilic displacement reaction conditions, and subsequent deprotection under the conditions as described above. Suzuki coupling of compound 12 with compound 4 using palladium catalyzed chemistry can be carried out as described above to give compound 13. Conversion of compound 13 to compounds of Formula I can be accomplished by Suzuki reaction with compound 6 using palladium catalyzed chemistry as described above. Alternatively, compound 13 can be made from compound 5 by a two-step process, wherein deprotection of compound 5 and then coupling with compound 9 is carried out using the conditions for from the conversion of compound 7 to compounds of Formula I as described above.

The Scheme 2 illustrate preparations of compounds of Formula IA and Formula IB, wherein R¹, R², A¹-A², X, Y¹, Y⁵, and Y⁶, and n are the same as those defined in Embodiment 1 above. Compounds 2A, 4, 6, 9 and 14 can be made by many methods known to the skilled person or are commercially available. The methods are readily apparent to the skilled person in view of the many methods known for making the requisite intermediates, so these methods are equally applicable to preparation of compounds with other embodiments. All palladium catalyzed reaction conditions, nucleophilic displacement reactions, deprotections, reduction aminations, amide or sulfonamide formation reactions, selectivity methods and approaches are the same as described for Scheme 1.

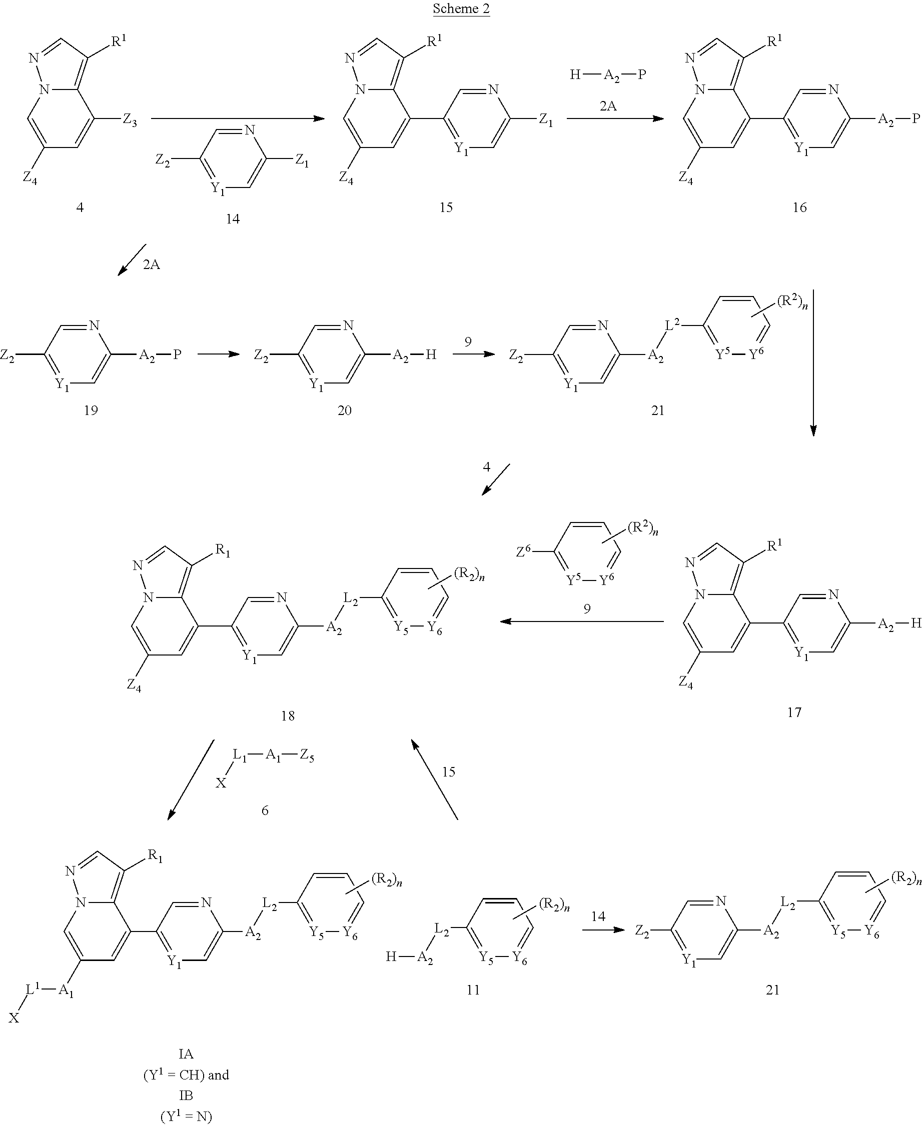

Thus, reaction of compound 4 with compound 14 undergoes Suzuki reaction using similar reaction conditions of palladium chemistry to provide compound 15. Conversion of compound 15 to compound 16 can be accomplished under Buchwald reaction conditions of palladium chemistry or nucleophilic displacement of Z₁ of compound 15 by compound 2A. Deprotection of compound 16 gives compound 17. Coupling of compound 17 with compound 9 under the similar conditions as described above provides compound 18. Reaction of compound 18 under Suzuki reaction conditions with compound 6 provides compounds of Formula IA and Formula IB. Compound 18 can be made alternatively from compound 14. Thus, conversion of compound 14 to compound 19 is accomplished under Buchwald reaction conditions of palladium chemistry or nucleophilic displacement reaction conditions by compound 2A. Compound 19 undergoes deprotection to provide compound 20, which undergoes coupling reaction conditions with compound 9 to give compound 21. Suzuki reaction of compound 21 with compound 4 provides compound 18. Alternatively, compound 18 can be made from reaction of compound 11 and compound 15 from Scheme 1 above under Buchwald reaction conditions of palladium chemistry or nucleophilic displacement reaction conditions as described above. Compound 21 can also be alternatively made from reaction of compound 11 and compound 14 under Buchwald reaction conditions of palladium chemistry or nucleophilic displacement reaction conditions as described above.

### EXAMPLES

The following examples illustrate certain embodiments of the present disclosure and how to make and use them. They are not intended to limit the scope of the invention. Those of skill in the art will readily recognize a variety of noncritical parameters and conditions which can be changed or modified to yield essentially the same results. The example compounds below were found to be inhibitors of RET according to one or more of the assays described herein.

In the following examples, the abbreviations below are used:
BINAP 2,2′-Bis(diphenylphosphino)-1,1′-binaphthyl
BOC tert-Butyloxycarbonyl
B₂(Pin)₂ Bis(pinacolato)diboron
BTEAC Benzyltriethylammonium chloride
CDI Carbonyldiimidazole
dba dibenzylideneacetone
DCE 1,2-Dichloroethene
DCM Dichloromethane
DHP Dihydropyran
DIAD Diisopropyl azodicarboxylate
DIPEA di-isopropylethylamine
DMA Dimethylacetamide
DMAP 4-Dimethylaminopyridine
DMF Dimethylformamide
DMSO Dimethylsulfoxide
dppf 1,1′Bis(diphenylphosphino) ferrocene
EDTA Ethylenediaminetetraacetic acid
EtOAc Ethyl acetate
EtOH Ethanol
HATU 1-[Bis(dimethylamino)methylene]-1H-1,2,3-triazolo[4,5-b]pyridinium 3-oxid hexafluorophosphate
KHMDS Potassium hexamethyldisilazane
LiHMDS Lithium hexamethyldisilazane
LG Leaving group
MeOH Methanol
MsCl Methanesulfonyl chloride
MTBE Methyl tert-butyl ether
Pd₂dba₃ Tris(dibenzylidenacetone) palladium
Pd(dppf)Cl₂ [1,1′Bis(diphenylphosphino) ferrocene]dichloropalladium (II)
PE Petroleum ether
PG Protecting group
PPTS Pyridinium p-toluenesulfonate
Prep-TLC Preparative Thin layer chromatography
PTSA p-toluenesulfonic acid
TBAF tetra-n-butylammonium fluoride
TBDMSCl t-Butyldimethylsilyl chloride
TEA Triethylamine
TES Triethylsilyl
TFA Trifluoacetic acid
Tf Triflyl
Tf₂O Trifluoromethanesulfonic anhydride
TLC Thin layer chromatography
THF Tetrahydrofuran
THP tetrahydropyran
TMS Trimethylsilyl
TosMIC Toluenesulfonylmethyl isocyanide
Xantphos 4,5-Bis(diphenylphosphino)-9,9-dimethylxanthene
XPhos 2-Dicyclohexylphosphino-2′,4′,6′-triisopropylbiphenyl

### Intermediate 1

### Preparation of 6-bromo-4-(6-(6-((6-methoxypyridin-3-yl)methyl)-3,6-diazabicyclo[3.1.1]heptan-3-yl)pyridin-3-yl) pyrazolo[1,5-a]pyridine-3-carbonitrile

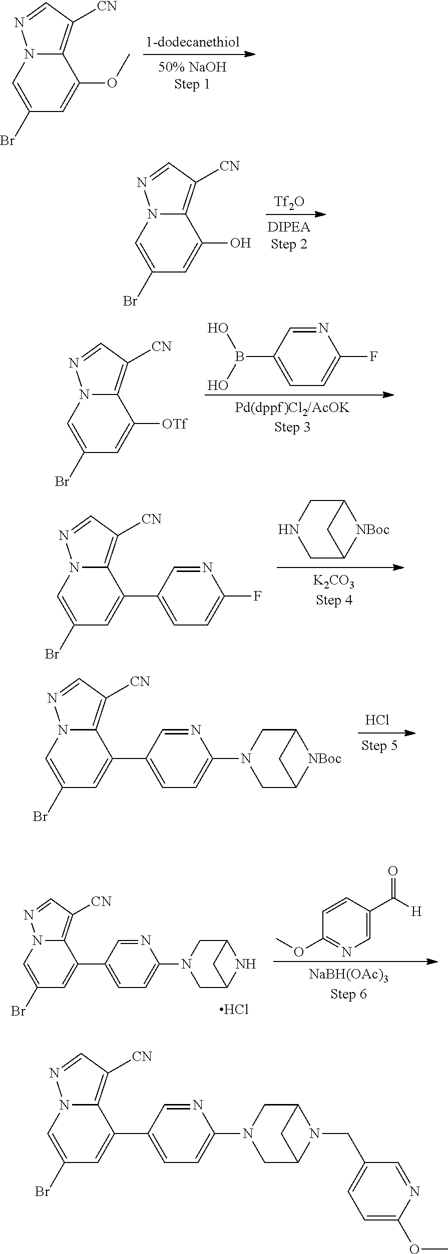

### Step 1. 6-bromo-4-hydroxypyrazolo[1,5-a]pyridine-3-carbonitrile

To a solution of 6-bromo-4-methoxypyrazolo[1,5-a]pyridine-3-carbonitrile (50 g, 198.4 mmol) in DMA (1 L) was added 50% NaOH (aqueous, 23.8 g, 297.6 mmol) and 1-dodecanethiol (60 g, 297.6 mmol). The mixture was stirred at 40° C. for 3 h, diluted with ice-water (4 L), acidified to pH 5˜6 by 10% AcOH (aqueous), and extracted with EtOAc (2 L×2). The combined extracts were washed with water (500 mL×2) and brine (500 mL), dried over Na₂SO₄, filtered off, and concentrated to give the title compound (44 g, yield: 93%).

### Step 2. 6-bromo-3-cyanopyrazolo[1,5-a]pyridin-4-yl trifluoromethanesulfonate

To a solution of the product of Step 1 above (44 g, 184.8 mmol) in DMA (500 mL) was added DIPEA (48 g, 369.7 mmol). The mixture was cooled in ice-water bath and a solution of N-phenyl-bis(trifluoromethanesulfonimide) (73 g, 203.3 mmol) was added slowly. After the addition was completed, the mixture was stirred at rt for 3 h, diluted with ice-water (3.5 L), and stirred for 0.5 h. The precipitate formed was collected by filtration, which was dissolved in EtOAc (1 L), washed with water (500 mL×3) and brine (500 mL), dried over Na₂SO₄, filtered off, and concentrated to give the title compound (67.9 g, yield: 99%).

### Step 3. 6-bromo-4-(6-fluoropyridin-3-yl) pyrazolo[1,5-a]pyridine-3-carbonitrile

To a solution of the product of Step 2 above (30 g, 81.1 mmol), (6-fluoropyridin-3-yl) boronic acid (11.42 g, 81.1 mmol) and AcOK (15.9 g, 162.1 mmol) in dioxane (300 mL/60 mL) was added Pd(dppf)Cl₂·DCM (1.32 g, 1.62 g) under N₂. The mixture was stirred at 35° C. overnight, cooled to rt, diluted with water (1000 mL). The reaction mixture was stirred for 30 min, and the precipitate was collected by filtration. The filter cake was dissolved in DCM/MeOH (10/1, 1000 mL), washed with water (500 mL) and brine (500 mL), dried over Na₂SO₄, filtered off, and concentrated. The residue was purified by flash column chromatography on silica gel (DCM/MeOH=10/1 to 100/1) to give the title compound (14.35 g, yield: 52%).

### Step 4. tert-butyl 3-(5-(6-bromo-3-cyanopyrazolo[1,5-a]pyridin-4-yl)pyridin-2-yl)-3,6-diazabicyclo[3.1.1]heptane-6-carboxylate

A mixture of the product of Step 3 above (7.62 g, 24.02 mmol), tert-butyl 3,6-diazabicyclo[3.1.1]heptane-6-carboxylate (5.71 g, 28.8 mmol) and K₂CO₃ (6.64 g, 48.04 mmol) in DMF (100 mL) was stirred at 110° C. overnight. The mixture was cooled to rt and concentrated. The residue was dissolved in EtOAc (500 mL), washed water (150 mL×2) and brine (150 mL), dried over Na₂SO₄, filtered off, and concentrated. The residue was purified by flash column chromatography on silica gel (PE/EtOAc=10/1 to 2/1) to give the title compound (7.0 g, yield: 59%).

### Step 5. 4-(6-(3,6-diazabicyclo[3.1.1]heptan-3-yl)pyridin-3-yl)-6-bromopyrazolo[1,5-a]pyridine-3-carbonitrile hydrochloride

To an ice-water cooled solution of the product of Step 4 above (7.0 g, 14.13 mmol) in DCM/MeOH (4/1, 110 mL) was added 4N HCl/dioxane (28 mL). The mixture was stirred at rt overnight before being concentrated to dryness to give the crude title compound (7.6, crude yield: 126%).

### Step 6. 6-bromo-4-(6-(6-((6-methoxypyridin-3-yl)methyl)-3,6-diazabicyclo[3.1.1]heptan-3-yl)pyridin-3-yl) pyrazolo[1,5-a]pyridine-3-carbonitrile

To a solution of the crude product of Step 5 above (1.12 g, 1.62 mmol) and 6-methoxynicotinaldehyde (335 mg, 2.44 mmol) in DCM (20 mL) was added NaBH(OAc)₃ (690 mg, 3.25 mmol). The reaction mixture was stirred at rt for 30 min. To the reaction mixture was added TEA (492 mg, 4.86 mmol). The mixture was stirred at rt overnight, quenched with saturated aqueous NaHCO₃ (20 mL). The mixture was extracted with DCM (100 mL). The extract was washed with water (50 mL) and brine (50 mL), dried over anhydrous Na₂SO₄, filtered off, and concentrated. The residue was purified by flash column chromatography on silica gel (MeOH/DCM=0 to 1/30) to give the title compound (650 mg, yield: 77%).

### Intermediate 2

### Preparation of 6-bromo-4-(5-(6-((6-methoxypyridin-3-yl)methyl)-3,6-diazabicyclo[3.1.1]heptan-3-yl) pyrazin-2-yl) pyrazolo[1,5-a]pyridine-3-carbonitrile

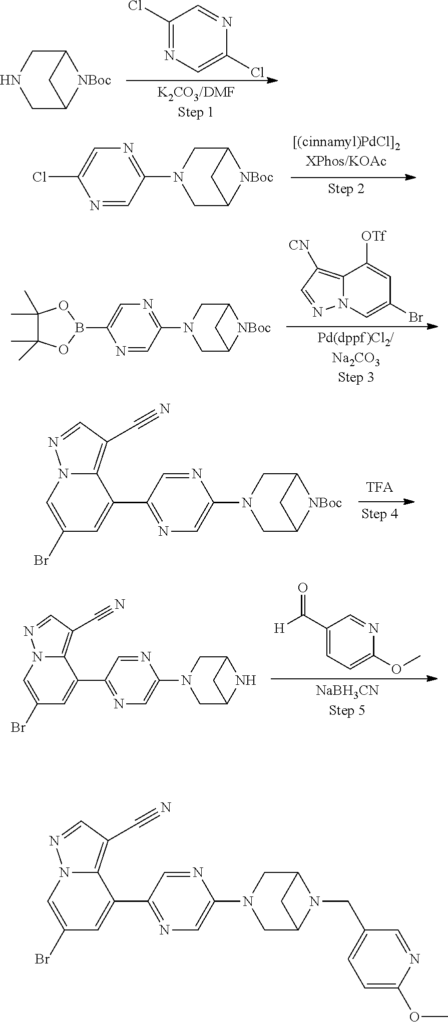

### Step 1. tert-butyl 3-(5-chloropyrazin-2-yl)-3,6-diazabicyclo[3.1.1]heptane-6-carboxylate

To a solution of tert-butyl 3,6-diazabicyclo[3.1.1]heptane-6-carboxylate (1.5 g, 10.1 mmol) in DMF (30 mL) was added K₂CO₃ (2.78 g, 20.1 mmol) and 2,5-dichloropyrazine (2.0 g, 10.1 mmol) successively. The mixture was stirred at 110° C. overnight, cooled to rt, and concentrated to dryness. The residue was taken up in EtOAc (300 mL), washed with water (100 mL) and brine (100 mL), dried over Na₂SO₄, filtered off, and concentrated. The residue was purified by flash column chromatography on silica gel (PE/EtOAc=10/1 to 5/1) to give the title compound (2.84 g, yield: 91%).

### Step 2. tert-butyl 3-(5-(4,4,5,5-tetramethyl-1,3,2-dioxaborolan-2-yl) pyrazin-2-yl)-3,6-diazabicyclo[3.1.1]heptane-6-carboxylate

To a solution of the product of Step 1 above (2.4 g, 7.72 mmol) in dry toluene (10 mL) was added B₂pin₂ (2.05 g, 8.11 mmol), XPhos (368 mg, 0.772 mmol), [(cinnamyl) PdCl]₂ (100 mg, 0.193 mmol) and KOAc (2.27 g, 23 mmol) successively. The mixture was stirred at 100° C. under nitrogen for 5 h, cooled to rt, and concentrated. The residue was used in the next step without any further purification.

### Step 3. tert-butyl 3-(5-(6-bromo-3-cyanopyrazolo[1,5-a]pyridin-4-yl) pyrazin-2-yl)-3,6-diazabicyclo[3.1.1]heptane-6-carboxylate

To a solution of the crude product of Step 2 above (7.72 mmol) in dioxane (20 mL) and water (4 mL) was added the product of Step 2 in the synthesis of Intermediate 1 (2.85 g, 7.72 mmol), Na₂CO₃ (1.63 g, 15.3 mmol), and Pd(dppf)Cl₂·DCM (630 mg, 0.772 mmol) successively. The mixture was stirred at 40° C. under nitrogen overnight and filtered off. The filtrate was diluted with DCM/MeOH (200 mL), washed with water (100 mL) and brine (100 mL), dried over Na₂SO₄, filtered off, and concentrated. The residue was purified by flash column chromatography on silica gel (PE/EtOAc=6/1 to 2/1) to give the title compound (729 mg, yield: 19%).

### Step 4. 4-(5-(3,6-diazabicyclo[3.1.1]heptan-3-yl) pyrazin-2-yl)-6-bromopyrazolo[1,5-a]pyridine-3-carbonitrile

To a solution of the product of Step 3 above (729 mg, 1.47 mmol) in DCM (18 mL) was added TFA at 0° C. The mixture was stirred at rt for 1 h before being concentrated to dryness. The residue was neutralized with saturated aqueous Na₂CO₃ and extracted with DCM/i-PrOH (3/1, 60 mL×3). The combined extracts were washed with water (50 mL) and brine (50 mL), dried over Na₂SO₄, filtered off, and concentrated to give the title compound (634 mg, crude, quan.), which was used in the next step without any further purification.

### Step 4. 6-bromo-4-(5-(6-((6-methoxypyridin-3-yl)methyl)-3,6-diazabicyclo[3.1.1]heptan-3-yl) pyrazin-2-yl) pyrazolo[1,5-a]pyridine-3-carbonitrile

To a solution of the crude product of Step 3 above (300 mg, 0.76 mmol) in MeOH (20 mL) was added 6-methoxynicotinaldehyde (155 mg, 1.14 mmol). The mixture was stirred at rt for 30 min before adding NaBH₃CN (96 mg, 1.52 mmol) and AcOH (0.2 mL, 3.5 mmol). The mixture was stirred at 50° C. overnight. The reaction mixture was concentrated and the residue was diluted with water (50 mL), neutralized with saturated aqueous NaHCO₃, extracted with DCM/MeOH (10/1, 100 mL), washed with water (50 mL) and brine (50 mL), dried over Na₂SO₄, filtered off, and concentrated. The residue was purified by flash column chromatography on silica gel (DCM/MeOH=100/1 to 30/1) to give the title compound (283 mg, yield: 72%).

### Example 1

### Preparation of 6-(5-(2-hydroxypropan-2-yl)pyridin-2-yl)-4-(5-(6-((6-methoxypyridin-3-yl)methyl)-3,6-diazabicyclo[3.1.1]heptan-3-yl) pyrazin-2-yl) pyrazolo[1,5-a]pyridine-3-carbonitrile

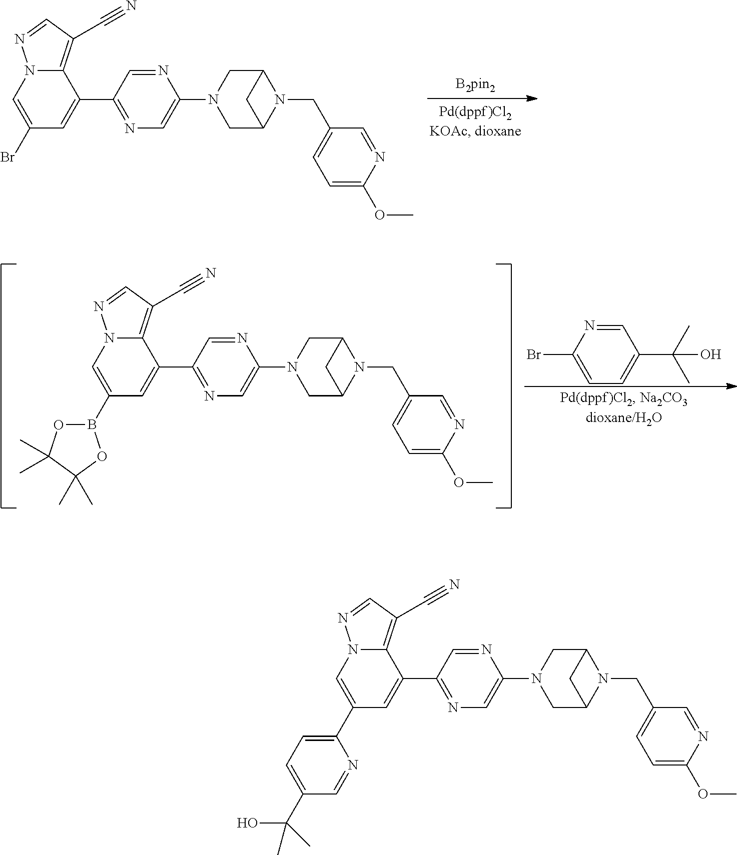

A mixture of Intermediate 2 (70 mg, 0.135 mmol), B₂Pin₂ (36 mg, 0.142 mmol), Pd(dppf)Cl₂·DCM (11 mg, 0.0135 mmol), and KOAc (26 mg, 0.27 mmol) in dioxane (1 mL) was stirred at 100° C. for 4 h under nitrogen. The mixture was cooled to rt, to which was added 2-(6-bromopyridin-3-yl) propan-2-ol (29 mg, 0.135 mmol), Na₂CO₃ (29 mg, 0.27 mmol), Pd(dppf)Cl₂·DCM (11 mg, 0.0125 mmol), dioxane (1 mL) and water (0.4 mL). The reaction mixture was stirred at 110° C. for 4 h under nitrogen, cooled to rt and concentrated. The residue was taken up in DCM/MeOH (10/1, 60 mL), washed with water (20 mL) and brine (20 mL), dried over anhydrous Na₂SO₄, filtered off, and concentrated. The residue was purified by reverse phase flash column chromatography (MeOH/H₂O=20% to 95%) to give the title compound (24 mg, yield: 32%). MS (ESI) m/z: 574.4 [M+1]+; ¹H NMR (400 MHz, DMSO-d6) δ 9.67 (s, 1H), 8.80 (s, 1H), 8.50 (d, J=8.7 Hz, 2H), 8.27 (s, 1H), 8.07 (s, 1H), 8.02 (d, J=9.0 Hz, 1H), 7.93 (d, J=8.8 Hz, 1H), 7.67 (d, J=8.1 Hz, 1H), 6.83 (d, J=8.7 Hz, 1H), 6.76 (d, J=8.3 Hz, 1H), 5.57 (s, 1H), 3.81 (s, 3H), 3.75 (d, J=11.7 Hz, 2H), 3.67 (d, J=5.4 Hz, 2H), 3.56 (d, J=9.0 Hz, 2H), 3.51 (s, 2H), 2.50 (s, 1H), 1.60 (s, 1H), 1.58 (s, 6H).

### Example 2

### Preparation of N-((1R,3S,5s,7s)-2-(5-(3-cyano-6-(5-(2-hydroxypropan-2-yl)pyridin-2-yl) pyrazolo[1,5-a]pyridin-4-yl) pyrazin-2-yl)-2-azaadamantan-5-yl)-6-methoxynicotinamide

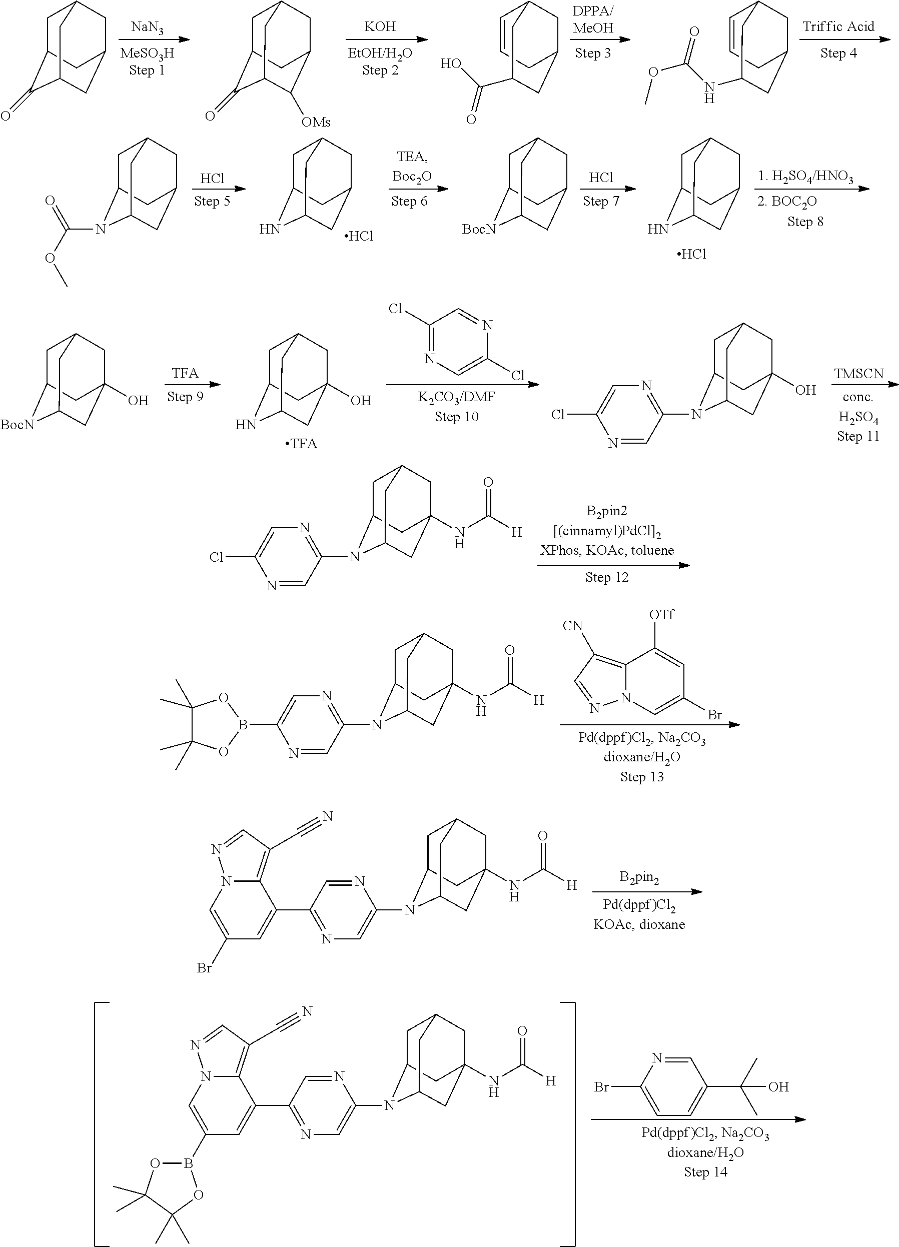

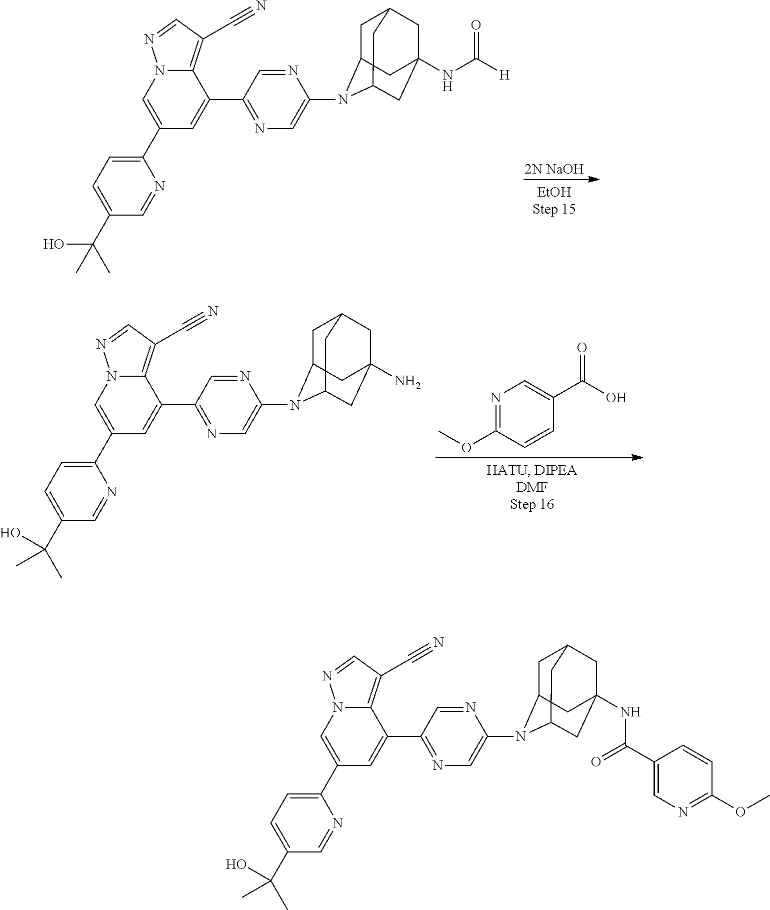 [SMILES: CC(C)(O)c1ccc(-c2cc(-c3cnc(N4C5CC6CC4CC(NC=O)(C6)C5)cn3)c3c(C#N)cnn3c2)nc1 ; "2N NaOH" ; CC(C)(O)c1ccc(-c2cc(-c3cnc(N4C5CC6CC4CC(N)(C6)C5)cn3)c3c(C#N)cnn3c2)nc1 ; COc1ccc(C(=O)O)cn1 ; COc1ccc(C(=O)NC23CC4CC(C2)N(c2cnc(-c5cc(-c6ccc(C(C)(C)O)cn6)cn6ncc(C#N)c56)cn2)C(C4)C3)cn1]

### Step 1. (1R,2S,3R,5S,7S)-4-oxoadamantan-2-yl methanesulfonate

To a solution of (1r,3r,5r,7r)-adamantan-2-one (50 g, 333 mmol) in MeSO₃H (416 g, 4329 mmol) was added portionwise NaN₃ (23 g, 351 mmol) over a period of 2 hours at 0° C. The reaction was stirred at rt for 3 days. The mixture was quenched with ice-water (2 L), and extracted with DCM/isopropanol (3/1, 2×3 L). The combined organic layers were washed with brine (1.5 L), dried over anhydrous Na₂SO₄, filtered off, and concentrated in vacuo to give the title compound (62 g, 62% yield).

### Step 2. bicyclo[3.3.1]non-6-ene-3-carboxylic acid

To a solution of the product of Step 1 above (62 g, 254 mmol) in EtOH (600 mL) and water (600 mL) was added KOH (43 g, 762 mmol). The mixture was heated to 110° C. overnight. After cooling to rt, the mixture was acidified with 1N HCl to pH 2. After removing the most ethanol in vacuo, the mixture was extracted with EtOAc (2×2 L). The combined organic layers were washed with brine (500 mL), dried over anhydrous Na₂SO₄, filtered off, and concentrated in vacuo to give the title compound (42 g, 99% yield).

### Step 3. methyl bicyclo[3.3.1]non-6-en-3-ylcarbamate

To a solution of the product of Step 2 above (42 g, 253 mmol) in toluene (400 mL) were added DPPA (76.5 g, 278 mmol) and TEA (38.3 g, 380 mmol). The mixture was stirred at 90° C. for 2 h under nitrogen atmosphere. After cooled to 0° C., to the mixture was added methanol (400 mL). The resulting mixture was heated to 100° C. overnight. The mixture was concentrated in vacuo and the residue was taken in EtOAc (2 L), washed with 1N HCl (500 mL), saturated aqueous NaHCO₃ (500 mL) and brine (500 mL), dried over anhydrous Na₂SO₄, filtered off, and concentrated in vacuo to give the title compound (20 g, 41% yield).

### Step 4. (1r,3r,5r,7r)-methyl 2-azaadamantane-2-carboxylate

To a solution of the product of Step 3 above (20 g, 102.5 mmol) in DCM (200 mL) was added triflic acid (77 g, 512 mmol) at 0° C. The mixture was stirred at rt overnight, quenched with ice-water (300 mL), extracted with DCM (2×500 mL). The combined organic layers were washed with saturated aqueous NaHCO₃ (200 mL) and brine (200 mL), dried over anhydrous Na₂SO₄, filtered off, and concentrated in vacuo to give the title compound (20 g, 100% yield).

### Step 5. (1r,3r,5r,7r)-2-azaadamantane hydrochloride

The product of Step 4 above (20 g, 102.5 mmol) was added to 4N HCl/dioxane (200 mL) and concentrated hydrochloric acid (200 mL) at 0° C. The mixture was stirred at 90° C. overnight and concentrated in vacuo to give the title compound (18 g, 100% yield).

### Step 6. (1r,3r,5r,7r)-tert-butyl 2-azaadamantane-2-carboxylate

To a solution of the product of Step 5 above (18 g, 103 mmol) in DCM (200 mL) was added TEA (31 g, 309 mmol) and Boc₂O (29 g, 134 mmol) at 0° C. The mixture was stirred at 0˜rt overnight. The mixture was diluted with DCM (300 mL), which was washed with water (100 mL) and brine (100 mL), dried over anhydrous Na₂SO₄, filtered off, and concentrated in vacuo. The residue was purified by flash column chromatography on silica gel (PE:EtOAc=50:1 to 20:1) to give the title compound (10 g, 41% yield).

### Step 7. (1r,3r,5r,7r)-2-azaadamantane hydrochloride

The product of Step 6 above (10 g, 102.5 mmol) was added to 4N HCl/dioxane (100 mL) at 0° C. The mixture was stirred at rt for 2 h. The mixture was concentrated in vacuo and the residue was triturated with hexane:ether (1:1, 50 mL×2) to give the title compound (4.8 g, 65% yield). LC-MS (m/z): 138.1

### Step 8. (1R,3S,5s,7s)-tert-butyl 5-hydroxy-2-azaadamantane-2-carboxylate

The product of Step 7 above (4.3 g, 24.7 mmol) was added to concentrated nitric acid (43 mL) and H₂SO₄ (7.2 mL) at 0° C. The mixture was stirred at 80° C. overnight. After cooling to rt, the mixture was quenched with ice-water (200 mL), and basified with solid Na₂CO₃. The aqueous layer was washed with DCM. The aqueous layer was diluted with THF (200 mL), cooled to 0° C., and treated with TEA (5 g, 49.4 mmol) and Boc₂O (7 g, 32.1 mmol). The resulting mixture was stirred at 0˜rt overnight and extracted with EtOAc (300 mL×2). The combined organic layers were washed with water (100 mL) and brine (100 mL), dried over anhydrous Na₂SO₄, filtered off, and concentrated in vacuo. The residue was purified by flash column chromatography on silica gel (PE:EtOAc=8:1 to 2:1) to give the title compound (2.47 g, 40% yield) as a colorless oil. ¹H NMR (400 MHz, CDCl₃) δ 4.46 (s, 2H), 2.29 (s, 1H), 1.79 (s, 2H), 1.73 (t, J=14.2 Hz, 4H), 1.67 (s, 1H), 1.64 (s, 1H), 1.61 (s, 2H), 1.53 (d, J=12.2 Hz, 2H), 1.48-1.40 (m, 9H).

### Step 9. (1R,3S,5s,7s)-2-azaadamantan-5-ol TFA salt

To a solution of the product of Step 8 above (2.47 g, 9.76 mmol) in DCM (30 mL) was added TFA (6 mL) at 0° C. The reaction was stirred at 0° C.˜rt for 4 h. The mixture was concentrated in vacuo and the residue was triturated with hexane:ether (1:1, 20 mL×2) to give the title compound (2.5 g, 100% yield).

### Step 10. (1R,3S,5s, 7s)-2-(5-chloropyrazin-2-yl)-2-azaadamantan-5-ol

To a solution of the product of Step 9 above (5.0 g, 20 mmol) in DMF (50 mL) was added K₂CO₃ (8.3 g, 60 mmol) and 2,5-dichloropyrazine (3.6 g, 24 mmol) successively. The mixture was stirred at 130° C. overnight, cooled to rt, and concentrated. The residue was taken up in EtOAc (600 mL), washed with water (200 mL) and brine (180 mL), dried over Na₂SO₄, filtered off, and concentrated. The residue was purified by flash column chromatography on silica gel (PE/EtOAc=2/1 to 1/1) to give the title compound (3.83 g, yield: 73%).

### Step 11. N-((1R,3S,5s, 7s)-2-(5-chloropyrazin-2-yl)-2-azaadamantan-5-yl) formamide

To a solution of the product of Step 10 above (3.6 g, 13.5 mmol) in concentrated H₂SO₄ (40 mL) was added TMSCN (9.1 g, 91.7 mmol) at 0° C. The mixture was stirred at 60° C. overnight, cooled to rt, poured slowly to crushed ice (500 g), basified with 5N NaOH to pH 10, and extracted with EtOAc (1 L×2). The combined extracts were washed with brine (500 mL), dried over Na₂SO₄, filtered off, and concentrated. The residue was purified by flash column chromatography on silica gel (PE/EtOAc=3/1 to 1/1) to give the title compound (2.62 g, yield: 57%).

### Step 12. N-((1R,3S,5s,7s)-2-(5-(4,4,5,5-tetramethyl-1,3,2-dioxaborolan-2-yl) pyrazin-2-yl)-2-azaadamantan-5-yl) formamide

To a solution of the product of Step 11 above (584 mg, 2.0 mmol) in dried toluene (10 mL) was added B₂pin₂ (533 mg, 2.1 mmol), XPhos (95 mg, 0.2 mmol), [(cinnamyl) PdCl]₂ (26 mg, 0.05 mmol) and KOAc (588 mg, 6.0 mmol) successively. The mixture was stirred at 100° C. under Nitrogen for 4 h. The mixture was cooled to rt, and concentrated. The residue was used in the next step without any further purification.

### Step 13. N-((1R,3S,5s,7s)-2-(5-(6-bromo-3-cyanopyrazolo[1,5-a]pyridin-4-yl) pyrazin-2-yl)-2-azaadamantan-5-yl) formamide

To a solution of the product of Step 12 above (2.0 mmol) in dioxane (20 mL) and water (2 mL) was added the product of Step 2 in Intermediate 1 (740 mg, 2.0 mmol), Na₂CO₃ (424 mg, 4.0 mmol), and Pd(dppf)Cl₂·DCM (163 mg, 0.2 mmol) successively. The mixture was stirred at 40° C. under nitrogen overnight. The reaction mixture was filtered and the filtrate was diluted with EtOAc (120 mL), washed with water (30 mL) and brine (30 mL), dried over Na₂SO₄, filtered off, and concentrated. The residue was purified by flash column chromatography on silica gel (PE/EtOAc=2/1 to DCM/EtOAc=1/1) to give the title compound (540 mg, yield: 47%).

### Step 14. N-((1R,3S,5s, 7s)-2-(5-(3-cyano-6-(5-(2-hydroxypropan-2-yl)pyridin-2-yl) pyrazolo[1,5-a]pyridin-4-yl) pyrazin-2-yl)-2-azaadamantan-5-yl) formamide

A mixture of the product of Step 13 above (90 mg, 0.189 mmol), B₂pin₂ (50 mg, 0.198 mmol), KOAc (37 mg, 0.378 mmol), and Pd(dppf)Cl₂·DCM (15 mg, 0.0189 mmol) in dioxane (1 mL) was stirred at 100° C. under nitrogen for 4 h. The mixture was cooled to rt, to which was added 2-(6-bromopyridin-3-yl) propan-2-ol (41 mg, 0.189 mmol), Pd(dppf)Cl₂·DCM (15 mg, 0.0189 mmol), Na₂CO₃ (40 mg, 0.378 mmol) and doxiane/H₂O (1 mL/0.4 mL). The mixture was stirred at 110° C. under nitrogen for 4 h. After cooling to rt, the mixture was diluted with DCM/MeOH (10/1, 60 mL), washed with water (20 mL) and brine (20 mL), dried over Na₂SO₄, filtered off, and concentrated. The residue was purified by prep-TLC (DCM/MeOH=15/1) to give the title compound (82 mg, yield: 81%).

### Step 15. 4-(5-((1R,3S,5s,7s)-5-amino-2-azaadamantan-2-yl) pyrazin-2-yl)-6-(5-(2-hydroxypropan-2-yl)pyridin-2-yl) pyrazolo[1,5-a]pyridine-3-carbonitrile

To a solution of the product of Step 14 above (82 mg, 0.153 mmol) in EtOH (3 mL) was added 2N NaOH (3 mL). The mixture was stirred at 60° C. for 3 h. After cooling to rt, the mixture was diluted with DCM (80 mL), washed with water (20 mL) and brine (20 mL), dried over Na₂SO₄, filtered off, and concentrated to give the title compound (73 mg, yield: 93%), which was used in the next step without any further purification.

### Step 16. N-((1R,3S,5s,7s)-2-(5-(3-cyano-6-(5-(2-hydroxypropan-2-yl)pyridin-2-yl) pyrazolo[1,5-a]pyridin-4-yl) pyrazin-2-yl)-2-azaadamantan-5-yl)-6-methoxynicotinamide

To a solution of the product of Step 15 above (73 mg, 0.144 mmol) in DMF (1 mL) was added 6-methoxynicotinic acid (22 mg, 0.144 mmol), HATU (82 mg, 0.216 mmol) and DIPEA (56 mg, 0.432 mmol) successively. The mixture was stirred at rt for 3 h, diluted with EtOAc (60 mL), washed with water (20 mL) and brine (20 mL), dried over Na₂SO₄, filtered off, and concentrated. The residue was purified by prep-TLC (DCM/MeOH=15/1) to give the title compound (40 mg, yield: 44%). MS (ESI) m/z: 642.4 [M+1]+; ¹H NMR (400 MHz, DMSO-d6) δ 9.55 (s, 1H), 8.82 (s, 1H), 8.75 (s, 1H), 8.65 (s, 1H), 8.59 (s, 1H), 8.46 (d, J=8.8 Hz, 2H), 8.19 (d, J=8.2 Hz, 1H), 8.06 (d, J=8.7 Hz, 1H), 7.98 (d, J=8.2 Hz, 1H), 7.86 (s, 1H), 6.84 (d, J=8.7 Hz, 1H), 5.29 (s, 1H), 4.93 (s, 2H), 3.88 (s, 3H), 2.35-2.24 (m, 4H), 2.21 (s, 1H), 2.15 (m, 2H), 1.84-1.75 (m, 4H), 1.50 (s, 6H).

### Example 3

### Preparation of 3-chloro-N-((1R,5S,6r)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl) picolinamide

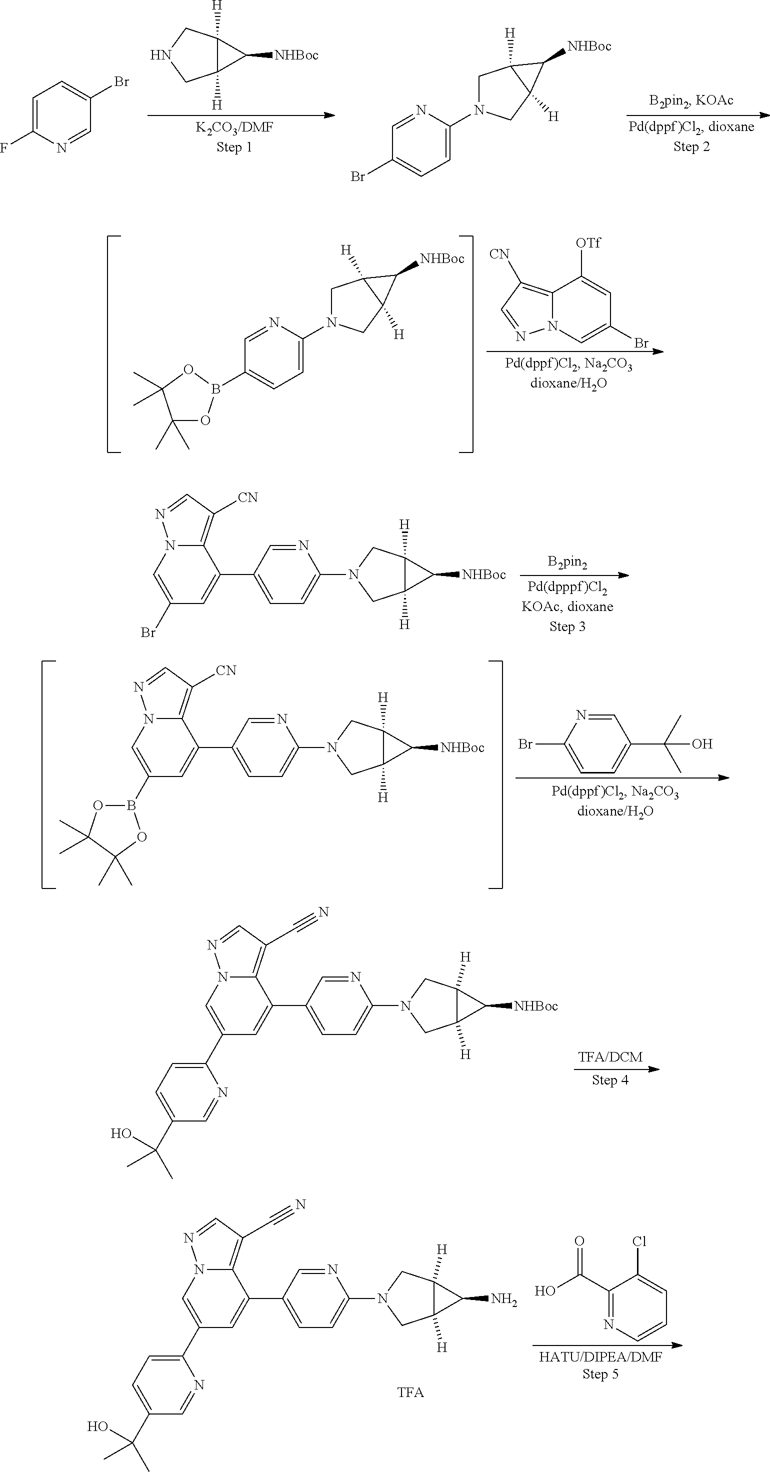

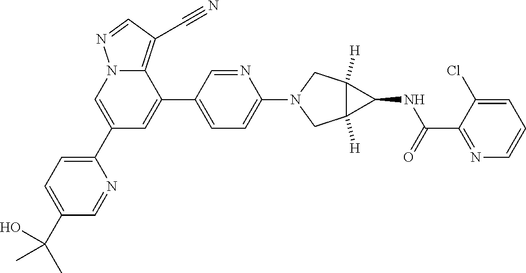 [SMILES: CC(C)(O)c1ccc(-c2cc(-c3ccc(N4C[C@@H]5[C@H](C4)[C@@H]5NC(=O)c4ncccc4Cl)nc3)c3c(C#N)cnn3c2)nc1]

### Step 1. tert-butyl ((1R,5S,6r)-3-(5-bromopyridin-2-yl)-3-azabicyclo[3.1.0]hexan-6-yl) carbamate

To a solution of 5-bromo-2-fluoropyridine (185 mg, 0.90 mmol) in DMF (5 mL) was added tert-butyl (1R,5S,6r)-3-azabicyclo[3.1.0]hexan-6-ylcarbamate (178 mg, 0.9 mmol), and K₂CO₃ (249 mg, 1.8 mmol). The reaction mixture was stirred at 110° C. overnight, cooled to rt, and concentrated. The residue was dissolved in EtOAc (100 mL), washed with H₂O (50 mL×2) and brine (50 mL), dried over anhydrous Na₂SO₄, filtered off, and concentrated in vacuo. The residue was purified by flash column chromatography on silica gel (PE/EtOAc=20/1 to 4/1) to give the title compound (108 mg, yield: 29%).

### Step 2. tert-butyl ((1R,5S,6r)-3-(5-(6-bromo-3-cyanopyrazolo[1,5-a]pyridin-4-yl)pyridin-2-yl)-3-azabicyclo[3.1.0]hexan-6-yl) carbamate

To a solution of the product of Step 1 above (108 mg, 0.305 mmol) in dioxane (1 mL) was added B₂Pin₂ (81 mg, 0.320 mmol), Pd(dppf)Cl₂·DCM (25 mg, 0.0305 mmol), and KOAc (60 mg, 0.61 mmol) at rt sequentially. The reaction mixture was flushed with nitrogen, and stirred at 100° C. for 4 h. After cooling to rt, to the reaction mixture was added the product of Step 2 in Intermediate 1 (113 mg, 0.305 mmol), Pd(dppf)Cl₂·DCM (25 mg, 0.0305 mmol), Na₂CO₃ (65 mg, 0.61 mmol), dioxane (1 mL) and water (1 mL). The resultant mixture was flushed with nitrogen, stirred at 40° C. overnight. The mixture was diluted with DCM/MeOH (10/1, 80 mL), washed with H₂O (30 mL×2) and brine (30 mL), dried over anhydrous Na₂SO₄, filtered off, and concentrated in vacuo. The residue was purified by prep-TLC on silica gel (PE/EtOAc=1/1) to give the title compound (129 mg, yield: 85%).

### Step 3. tert-butyl ((1R,5S,6r)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl) carbamate

A mixture of the product of Step 2 (129 mg, 0.26 mmol), B₂pin₂ (69 mg, 0.273 mmol), KOAc (51 mg, 0.52 mmol), and Pd(dppf)Cl₂·DCM (21 mg, 0.026 mmol) in dioxane (1 mL) was stirred at 100° C. under nitrogen for 4 h. The mixture was cooled to rt, to which was added 2-(6-bromopyridin-3-yl) propan-2-ol (41 mg, 0.189 mmol), Pd(dppf)Cl2·DCM (21 mg, 0.026 mmol), Na₂CO₃ (55 mg, 0.52 mmol) dioxiane/H₂O (1 mL/0.4 mL). The resulting mixture was stirred at 110° C. under nitrogen for 4 h. After cooling to rt, the mixture was diluted with DCM/MeOH (10/1, 80 mL), washed with water (20 mL) and brine (20 mL), dried over Na₂SO₄, filtered off, and concentrated. The residue was purified reverse phase flash column chromatography on C18 (MeOH/H₂O) to give the title compound (66 mg, yield: 46%).

### Step 4. 4-(6-((1R,5S,6r)-6-amino-3-azabicyclo[3.1.0]hexan-3-yl)pyridin-3-yl)-6-(5-(2-hydroxypropan-2-yl)pyridin-2-yl) pyrazolo[1,5-a]pyridine-3-carbonitrile trifluoroacetate

To a solution of the product of Step 3 above (66 mg, 0.12 mmol) in DCM (6 mL) was added TFA (2 mL) at 0° C. The mixture was stirred at rt for 1 h and concentrated to give the title compound (97 mg, crude), which was used in the next step without any further purification.

### Step 5. 3-chloro-N-((1R,5S,6r)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl) picolinamide

To a solution of the product of Step 4 above (97 mg, 0.12 mmol) in DMF (3 mL) was added 3-chloropicolinic acid (19 mg, 0.12 mmol), HATU (68 mg, 0.18 mmol) and DIPEA (78 mg, 0.6 mmol) successively. The mixture was stirred at rt for 3 h, diluted with DCM/MeOH (10/1, 60 mL), washed with water (20 mL) and brine (20 mL), dried over Na₂SO₄, filtered off, and concentrated. The residue was purified by prep-TLC (EtOAc/MeOH=20/1) to give the title compound (43 mg, yield: 61%). MS (ESI) m/z: 591.3 [M+1]+; ¹H NMR (400 MHz, DMSO-d6) δ 9.51 (s, 1H), 8.81 (s, 1H), 8.72 (s, 1H), 8.47 (s, 1H), 8.40 (d, J=4.6 Hz, 1H), 8.33 (s, 1H), 8.16 (d, J=8.2 Hz, 1H), 8.09 (s, 1H), 7.97 (d, J=8.3 Hz, 1H), 7.89 (d, J=8.1 Hz, 1H), 7.77 (d, J=8.7 Hz, 1H), 7.42 (dd, J=8.2, 4.6 Hz, 1H), 6.50 (d, J=8.7 Hz, 1H), 5.29 (s, 1H), 3.81-3.58 (m, 4H), 2.96 (t, J=7.0 Hz, 1H), 2.11 (d, J=7.0 Hz, 2H), 1.49 (s, 6H).

### Example 4

### Preparation of 6-(5-(2-hydroxypropan-2-yl)-1-methyl-1H-pyrazol-3-yl)-4-(6-(6-((6-methoxypyridin-3-yl)methyl)-3,6-diazabicyclo[3.1.1]heptan-3-yl)pyridin-3-yl) pyrazolo[1,5-a]pyridine-3-carbonitrile

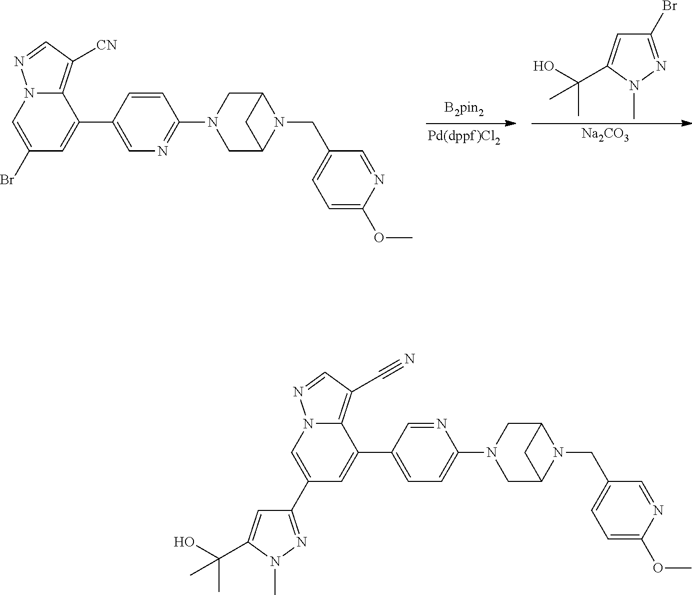

A mixture of Intermediate 1 (100 mg, 0.193 mmol), B₂Pin₂ (49 mg, 0.193 mmol), Pd (dppf) C12·DCM (18.8 mg, 0.0193 mmol), and KOAc (56.7 mg, 0.58 mmol) in dioxane (1 mL) was stirred at 100° C. for 3 h under N₂. After cooling to rt, to the mixture was added 2-(3-bromo-1-methyl-1H-pyrazol-5-yl) propan-2-ol (29 mg, 0.135 mmol), Na₂CO₃ (40 mg, 0.38 mmol), and water (0.2 mL). The reaction mixture was stirred at 100° C. for 3 h under N₂, cooled to rt. The mixture was concentrated. The residue was taken up in DCM/MeOH (10/1, 20 mL), washed with water (10 mL) and brine (10 mL), dried over anhydrous Na₂SO₄, filtered off, and concentrated. The residue was purified by reverse phase flash column chromatography (MeOH/H₂O=20% to 95%) to give the title compound (22 mg, yield: 28%). MS (ESI) m/z: 576.6 [M+1]+; ¹H NMR (400 MHz, CDCl₃) δ 8.82 (s, 1H), 8.39 (s, 1H), 8.28 (s, 1H), 8.11 (s, 1H), 7.81 (d, J=8.7 Hz, 1H), 7.76 (s, 1H), 7.65 (s, 1H), 6.75-6.66 (m, 2H), 6.40 (s, 1H), 4.14 (s, 3H), 3.92 (s, 3H), 3.88-3.75 (m, 4H), 3.68-3.56 (m, 4H), 2.71 (s, 1H), 1.70 (s, 6H), 1.66 (d, 1H).

### Example 5

### Preparation of 3-chloro-N-(((1R,5S,6s)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl)methyl) picolinamide

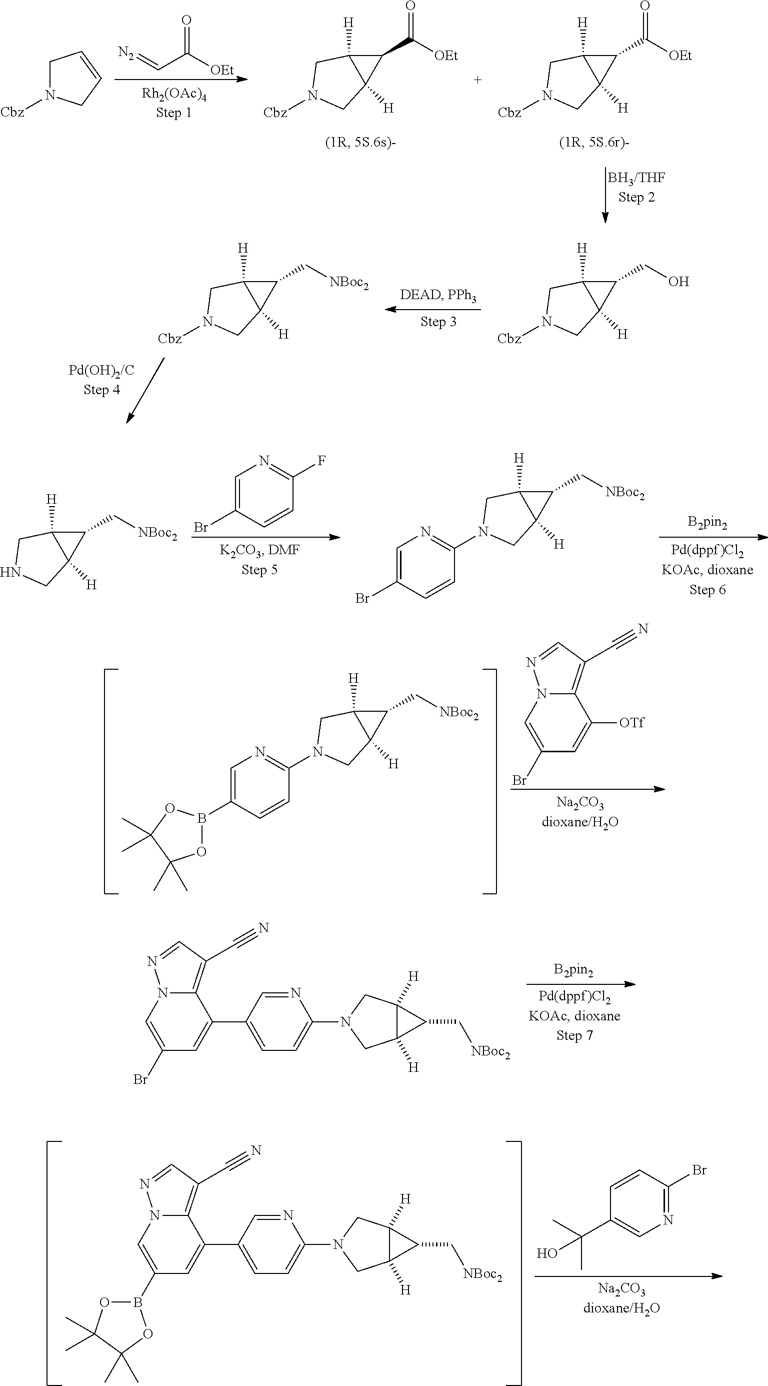

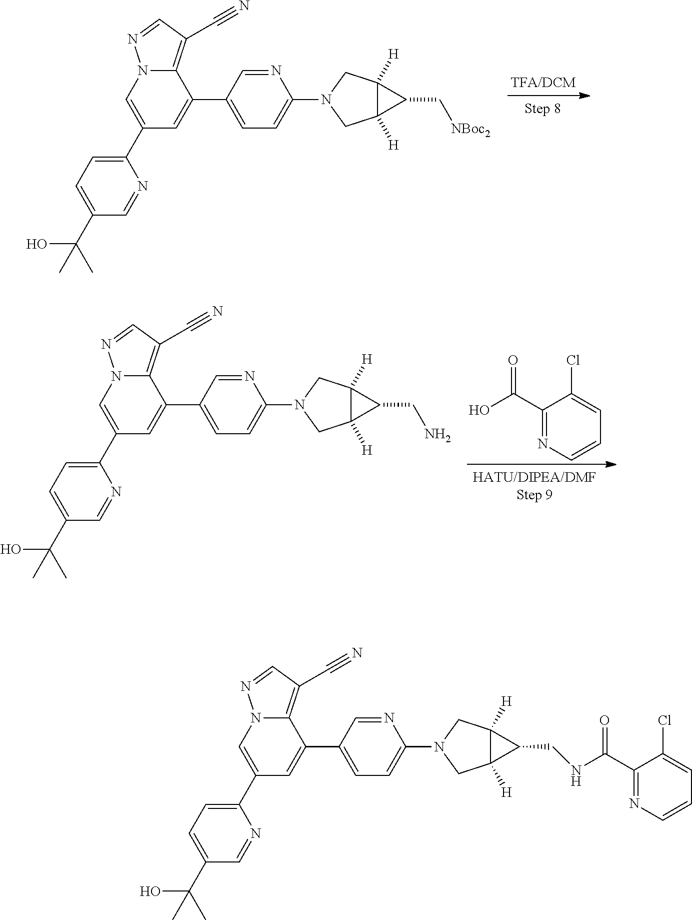

### Step 1. 3-benzyl 6-ethyl (1R,5S,6r)-3-azabicyclo[3.1.0]hexane-3,6-dicarboxylate and 3-benzyl 6-ethyl (1R,5S,6s)-3-azabicyclo[3.1.0]hexane-3,6-dicarboxylate

To a solution of benzyl 2,5-dihydro-1H-pyrrole-1-carboxylate (5.0 g, 24.6 mmol) and Rh₂(OAc)₄ (500 mg, 1.13 mmol) in DCE (50 mL) heated to 80° C. was added a solution of ethyl 2-diazoacetate (14 g, 123 mmol) in DCE (50 ml) was added dropwise over a period of 4 h. After the addition is completed, the mixture was stirred at 80° C. overnight. After cooling, the mixture was concentrated in vacuo. The residue was purified by flash column chromatography on silica gel (PE/EtOAc=20/1 to 4/1) to give (1R,5S,6r)-isomer (upper spot on TLC, 3.1 g, yield: 43%) and (1R,5S,6s)-isomer (lower spot on TLC, 1.6 g, yield: 22%).

### Step 2. benzyl (1R,5S,6r)-6-(hydroxymethyl)-3-azabicyclo[3.1.0]hexane-3-carboxylate

To a solution of the (1R,5S,6r)-isomer of Step 1 above in THF (25 mL) was added dropwise BH₃/THF (1 N, 18 mL, 18 mmol) at 0° C. After the addition was completed, the mixture was heated to 70° C., stirred for 2 h. The mixture was concentrated in vacuo and the residue was taken up in DCM (50 mL) and brine (30 mL) and the layers were separated. The aqueous layer was acidified to pH 5 with 1N HCl and extracted with DCM (50 mL×2). The combined organic layers were dried over anhydrous Na₂SO₄ and concentrated in vacuo. The residue was purified by flash column chromatography on silica gel (PE/EtOAc=1/1) to give the title compound (1.18 g, yield: 47%).

### Step 3. benzyl (1R,5S,6r)-6-((bis(tert-butoxycarbonyl)amino)methyl)-3-azabicyclo[3.1.0]hexane-3-carboxylate

To a solution of the product of Step 2 above (1.13 g, 4.57 mmol), di-tert-butyl iminodicarboxylate (1.09 g, 5.03 mmol) and PPh₃ (1.56 g, 5.94 mmol) in THF (20 mL) was added dropwise DEAD (1.03 g, 5.94 mmol) at 0° C. under N₂. The mixture was allowed to warm to rt, heated to 50° C. and stirred overnight. The mixture was extracted with EtOAc (100 mL). The organic layer was washed with H₂O (30 mL) and brine (30 mL), dried over anhydrous Na₂SO₄, filtered off, and concentrated in vacuo. The residue was purified by flash column chromatography on silica gel (PE/EtOAc=12/1 to 8/1) to give the title compound (900 mg, 42%).

### Step 4. tert-butyl (((1R,5S,6r)-3-azabicyclo[3.1.0]hexan-6-yl)methyl) (tert-butoxycarbonyl) carbamate

To a solution of the product of Step 3 above (900 mg, 2.02 mmol) in MeOH (15 mL) was added Pd(OH)₂/C (100 mg, 20% on carbon, ca. 50% H₂O). The mixture was stirred at rt for 1.5 h over a hydrogen balloon. The mixture was filtered off and the filtrate was concentrated to give the title compound (616 mg, yield: 98%).

### Step 5. tert-butyl N-(tert-butoxycarbonyl)-N-(((1R,5S,6r)-3-(5-bromopyridin-2-yl)-3-azabicyclo[3.1.0]hexan-6-yl)methyl) carbamate

A mixture of the product of Step 4 above (560 mg, 1.79 mmol), 5-bromo-2-fluoropyridine (316 mg, 1.79 mmol) and K₂CO₃ (494 mg, 3.58 mmol) was stirred at 100° C. overnight. After cooling to rt, the mixture was concentrated in vacuo. The residue was purified by flash column chromatography on silica gel (PE/EtOAc=30/1 to 15/1) to give the title compound (550 mg, yield: 60%).

### Step 6. tert-butyl (((1R,5S,6r)-3-(5-(6-bromo-3-cyanopyrazolo[1,5-a]pyridin-4-yl)pyridin-2-yl)-3-azabicyclo[3.1.0]hexan-6-yl)methyl) (tert-butoxycarbonyl) carbamate

A solution of the product of Step 5 above (381 mg, 0.813 mmol), B₂Pin₂ (217 mg, 0.854 mmol), Pd(dppf)Cl₂·DCM (66 mg, 0.0813 mmol), and KOAc (160 mg, 1.626 mmol) in dioxane (10 mL) was stirred at 100° C. for 4 h under nitrogen. To the mixture after cooling to rt was added the product of Step 2 in Intermediate 1 (301 mg, 0.813 mmol), Na₂CO₃ (172 mg, 1.626 mmol), Pd(dppf)Cl₂·DCM (66 mg, 0.0813 mmol) and dioxane/H₂O (5 mL/3 mL). The reaction mixture was stirred at 40° C. for 8 h, cooled to rt, diluted with DCM/MeOH (10/1, 120 mL), washed with H₂O (50 mL) and brine (50 mL), dried over anhydrous Na₂SO₄, filtered off and concentrated in vacuo. The residue was purified by flash column chromatography on silica gel (PE/EtOAc=10/1 to 3/1) to give the title compound (332 mg, yield: 53%).

### Step 7. tert-butyl N-(tert-butoxycarbonyl)-N-(((1R,5S,6s)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl)methyl) carbamate

A mixture of the product of Step 6 (160 mg, 0.263 mmol), B₂pin₂ (60 mg, 0.276 mmol), KOAc (52 mg, 0.526 mmol), and Pd(dppf)Cl₂·DCM (21 mg, 0.0263 mmol) in dioxane (3 mL) was stirred at 100° C. under nitrogen for 4 h. The mixture was cooled to rt, to which was added 2-(6-bromopyridin-3-yl) propan-2-ol (57 mg, 0.263 mmol), Pd(dppf)Cl₂·DCM (21 mg, 0.0263 mmol), Na₂CO₃ (56 mg, 0.526 mmol) and dioxiane/H₂O (1 mL/0.4 mL). The resulting mixture was stirred at 110° C. under nitrogen for 4 h. After cooling to rt, the mixture was diluted with DCM/MeOH (10/1, 100 mL), washed with water (20 mL) and brine (20 mL), dried over Na₂SO₄, filtered off, and concentrated. The residue was purified by prep-TLC (DCM/MeOH=20/1) to give the title compound (55 mg, yield: 31%).

### Step 8. 4-(6-((1R,5S,6s)-6-(aminomethyl)-3-azabicyclo[3.1.0]hexan-3-yl)pyridin-3-yl)-6-(5-(2-hydroxypropan-2-yl)pyridin-2-yl) pyrazolo[1,5-a]pyridine-3-carbonitrile

To a solution of the product of Step 7 above (55 mg, 0.083 mmol) in DCM (6 mL) was added TFA (2 mL) at 0° C. The mixture was stirred at rt for 1 h and concentrated to give the title compound (90 mg, crude), which was used in the next step without any further purification.

### Step 9. 3-chloro-N-(((1R,5S,6s)-3-(5-(3-cyano-6-(5-(2-hydroxypropan-2-yl)pyridin-2-yl) pyrazolo[1,5-a]pyridin-4-yl)pyridin-2-yl)-3-azabicyclo[3.1.0]hexan-6-yl)methyl) picolinamide

To a solution of the product of Step 8 above (90 mg, 0.083 mmol) in DMF (3 mL) was added 3-chloropicolinic acid (13 mg, 0.083 mmol), HATU (47 mg, 0.125 mmol) and DIPEA (53 mg, 0.415 mmol) successively. The mixture was stirred at rt for 3 h, purified by reverse phase column flash chromatography (H₂O/MeOH=80/20 to 10/90) to give the title compound (31 mg, yield: 63%). MS (ESI) m/z: 605.4 [M+1]+; ¹H NMR (400 MHz, DMSO-d6) δ 9.51 (s, 1H), 8.80 (s, 1H), 8.76 (t, J=5.5 Hz, 1H), 8.73 (s, 1H), 8.55 (d, J=4.5 Hz, 1H), 8.35 (s, 1H), 8.16 (d, J=8.3 Hz, 1H), 8.13 (s, 1H), 8.03 (d, J=8.2 Hz, 1H), 7.96 (d, J=8.3 Hz, 1H), 7.79 (d, J=8.7 Hz, 1H), 7.53 (dd, J=8.1, 4.6 Hz, 1H), 6.59 (d, J=8.7 Hz, 1H), 5.29 (s, 1H), 3.74 (d, J=10.5 Hz, 2H), 3.45 (d, J=8.9 Hz, 2H), 3.27 (t, J=5.4 Hz, 2H), 1.75 (s, 2H), 1.49 (s, 6H), 0.93 (s, 1H).

### Example 6

### Preparation of 3-chloro-N-((3aR,5s,6aS)-2-(5-(3-cyano-6-(5-(2-hydroxypropan-2-yl)pyridin-2-yl) pyrazolo[1,5-a]pyridin-4-yl) pyrazin-2-yl)-5-methyloctahydrocyclopenta[c]pyrrol-5-yl) picolinamide

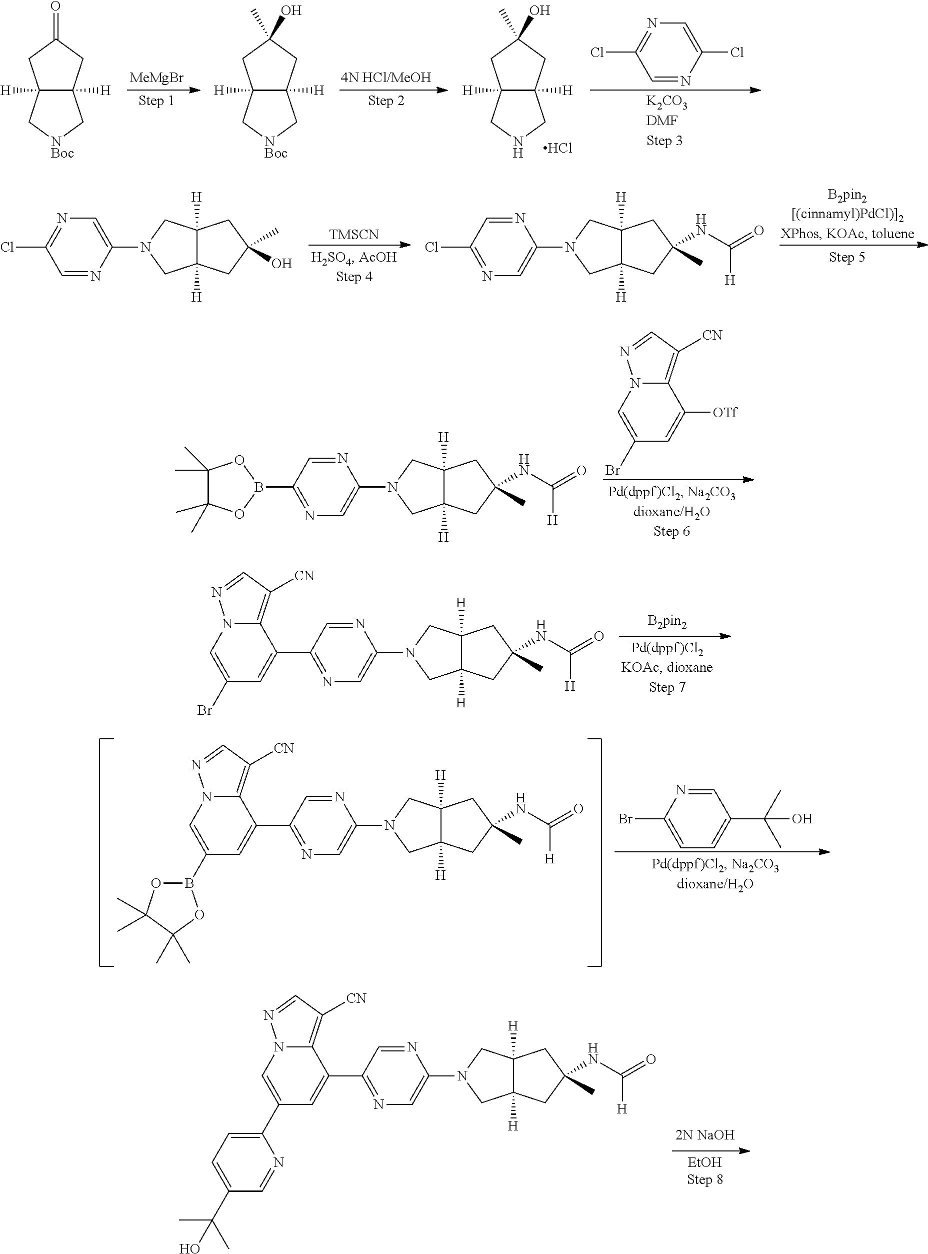

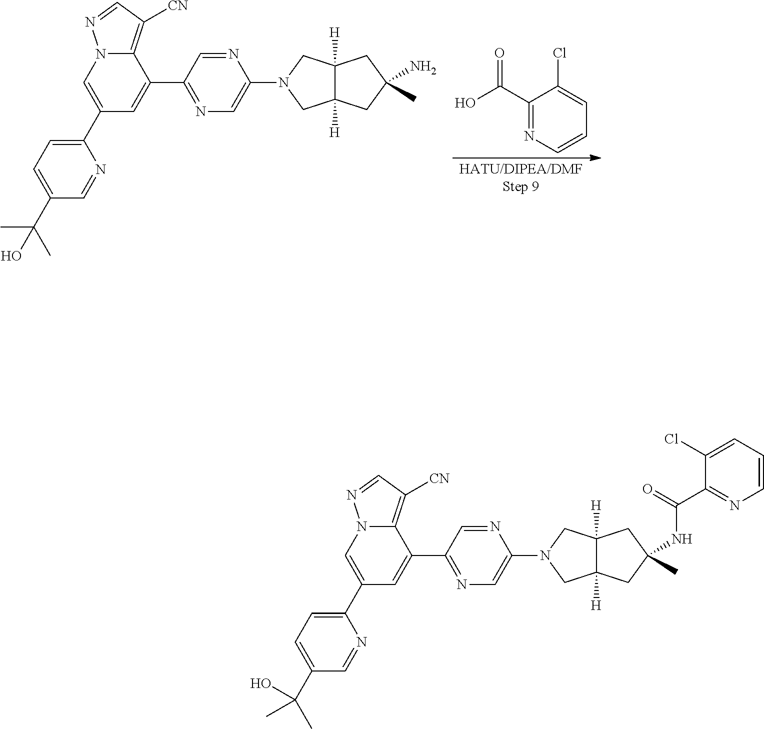 [SMILES: CC(C)(O)c1ccc(-c2cc(-c3cnc(N4C[C@@H]5C[C@@](C)(N)C[C@@H]5C4)cn3)c3c(C#N)cnn3c2)nc1 ; O=C(O)c1ncccc1Cl ; CC(C)(O)c1ccc(-c2cc(-c3cnc(N4C[C@@H]5C[C@@](C)(NC(=O)c6ncccc6Cl)C[C@@H]5C4)cn3)c3c(C#N)cnn3c2)nc1]

### Step 1. (3aR,5r,6aS)-tert-butyl 5-hydroxy-5-methylhexahydrocyclopenta[c]pyrrole-2 (1H)-carboxylate

To a solution of (3aR,6aS)-tert-butyl 5-oxohexahydrocyclopenta[c]pyrrole-2 (1H)-carboxylate (2.25 g, 10 mmol) in dry toluene (25 mL) was added methylmagnesium bromide (1.0 N, 25 mmol) at −30° C. The mixture was stirred at −30° C. for 2 h. The reaction was quenched by dropwise addition of MeOH (2 mL) and HCl (6 N, 10 mL) at −30° C. The mixture was diluted with EtOAc (100 mL), washed by H₂O (30× 2 mL) and brine (30 mL), dried over anhydrous Na₂SO₄, filtered off and concentrated in vacuo. The residue was purified by flash column chromatography on silica gel (PE/EtOAc=4/1-1/1) to give the title compound (2.0 g, yield: 83%).

### Step 2. (3aR,5r,6aS)-5-methyloctahydrocyclopenta[c]pyrrol-5-ol hydrochloride

A solution of the product of Step 1 above (1.5 g, 6.22 mmol) in HCl/MeOH (4 N, 10 mL) was stirred at 40° C. for 2 h. The reaction mixture was concentrated and dried in vacuo to give the crude title compound (quantitatively).

### Step 3. (3aR,5r,6aS)-2-(5-chloropyrazin-2-yl)-5-methyloctahydrocyclopenta[c]pyrrol-5-ol

To a solution of the product of Step 2 above (610 mg, 4.1 mmol) and K₂CO₃ (1.7 g, 12.3 mmol) in DMF (5 mL) was added 2,5-dichloropyrazine (0.8 g, 4.5 mmol). The mixture was stirred at 110° C. for 2 h. After cooling to rt, the mixture was diluted with EtOAc (100 mL), washed by H₂O (30 mL×2) and brine (30 mL), dried over anhydrous Na₂SO₄, filtered off, and concentrated in vacuo. The residue was purified by flash column chromatography on silica gel (PE/EtOAc=4/1 to 1/1) to give the title compound (630 mg, yield: 61%).

### Step 4. N-((3aR,5s,6aS)-2-(5-chloropyrazin-2-yl)-5-methyloctahydrocyclopenta[c]pyrrol-5-yl) formamide

To a solution of the product of Step 3 above (200 mg, 0.79 mmol) and TMSCN (234 mg, 2.36 mmol) in HOAc (0.5 mL) was added concentrated H₂SO₄ (0.4 mL) at 0° C. The mixture was stirred at rt for 2 h. The reaction was quenched with ice, basified with aqueous NaOH (5 N) to pH 8-9, and extracted with DCM (50 mL×3). The combined extracts were washed with H₂O (30 mL×2) and brine (30 mL), dried over anhydrous Na₂SO₄, filtered off, and concentrated in vacuo. The residue was purified by Prep-TLC (PE/EtOAc=1/1 to EtOAc) to give the title compound (215 mg, yield: 97%).

### Step 5. N-((3aR,5s,6aS)-5-methyl-2-(5-(4,4,5,5-tetramethyl-1,3,2-dioxaborolan-2-yl) pyrazin-2-yl) octahydrocyclopenta[c]pyrrol-5-yl) formamide

To a solution of the product of Step 4 above (651 mg, 2.32 mmol) in dried toluene (10 mL) was added B₂pin₂ (609 mg, 2.4 mmol), XPhos (95 mg, 0.2 mmol), [(cinnamyl)PdCl]₂ (26 mg, 0.05 mmol) and KOAc (588 mg, 6.0 mmol) successively. The mixture was stirred at 100° C. under nitrogen for 4 h, cooled to rt, and concentrated. The residue was used in the next step without any further purification.

### Step 6. N-((3aR,5s,6aS)-2-(5-(6-bromo-3-cyanopyrazolo[1,5-a]pyridin-4-yl) pyrazin-2-yl)-5-methyloctahydrocyclopenta[c]pyrrol-5-yl) formamide

To a solution of the product of Step 5 above (2.0 mmol) in dioxane (10 mL) and water (2 mL) was added the product of Step 2 in Intermediate 1 (858 mg, 2.32 mmol), Na₂CO₃ (492 mg, 4.64 mmol), and Pd(dppf)Cl₂·DCM (109 mg, 0.232 mmol) successively. The mixture was stirred at 40° C. under nitrogen overnight, cooled to rt, diluted with DCM/MeOH (10/1, 200 mL), washed with water (50 mL) and brine (50 mL), dried over Na₂SO₄, filtered off, and concentrated. The residue was purified by prep-TLC (PE/EtOAc=1/4) to give the title compound (153 mg, yield: 15%).

### Step 7. N-((3aR,5s,6aS)-2-(5-(3-cyano-6-(5-(2-hydroxypropan-2-yl)pyridin-2-yl) pyrazolo[1,5-a]pyridin-4-yl) pyrazin-2-yl)-5-methyloctahydrocyclopenta[c]pyrrol-5-yl) formamide

A mixture of the product of Step 6 above (153 mg, 0.328 mmol), B₂pin₂ (87 mg, 0.344 mmol), KOAc (64 mg, 0.656 mmol), and Pd(dppf)Cl₂·DCM (27 mg, 0.0328 mmol) in dioxane (2 mL) was stirred at 100° C. under nitrogen for 4 h. The mixture was cooled to rt, to which was added 2-(6-bromopyridin-3-yl) propan-2-ol (71 mg, 0.328 mmol), Pd(dppf)Cl₂·DCM (27 mg, 0.0328 mmol), Na₂CO₃ (70 mg, 0.656 mmol) and doxiane/H₂O (3 mL/1 mL). The mixture was stirred at 110° C. under nitrogen for 4 h. After cooling to rt, the mixture was diluted with DCM/MeOH (10/1, 60 mL), washed with water (20 mL) and brine (20 mL), dried over Na₂SO₄, filtered off, and concentrated. The residue was purified by prep-TLC (DCM/MeOH=20/1) to give the title compound (53 mg, yield: 31%).

### Step 8. 4-(5-((3aR,5s,6aS)-5-amino-5-methylhexahydrocyclopenta[c]pyrrol-2 (1H)-yl) pyrazin-2-yl)-6-(5-(2-hydroxypropan-2-yl)pyridin-2-yl) pyrazolo[1,5-a]pyridine-3-carbonitrile

To a solution of the product of Step 7 above (53 mg, 0.101 mmol) in EtOH (2 mL) was added 2N NaOH (2 mL). The mixture was stirred at 60° C. for 3 h. After cooling to rt, the mixture was diluted with DCM/IPA (3/1, 80 mL), washed with water (20 mL) and brine (20 mL), dried over Na₂SO₄, filtered off, and concentrated to give the title compound (50 mg, crude), which was used in the next step without any further purification.

### Step 9. 3-chloro-N-((3aR,5s,6aS)-2-(5-(3-cyano-6-(5-(2-hydroxypropan-2-yl)pyridin-2-yl) pyrazolo[1,5-a]pyridin-4-yl) pyrazin-2-yl)-5-methyloctahydrocyclopenta[c]pyrrol-5-yl) picolinamide

To a solution of the product of Step 8 above (50 mg, ˜0.101 mmol) in DMF (1 mL) was added 3-chloropicolinic acid (16 mg, 0.101 mmol), HATU (58 mg, 0.152 mmol) and DIPEA (39 mg, 0.303 mmol) successively. The mixture was purified by reverse phase flash column chromatography (MeOH/H₂O=5%˜95%) to give the title compound (30 mg, yield: 48%). MS (ESI) m/z: 634.4 [M+1]+; ¹H NMR (400 MHz, DMSO-d6) δ 9.55 (s, 1H), 8.82 (s, 1H), 8.74 (s, 1H), 8.62 (d, J=8.2 Hz, 1H), 8.54-8.47 (m, 1H), 8.42 (s, 1H), 8.29 (s, 1H), 8.16 (t, J=12.8 Hz, 2H), 8.03-7.92 (m, 2H), 7.48 (m, 1H), 5.30 (s, 1H), 3.75-3.61 (m, 2H), 3.55 (d, J=10.7 Hz, 2H), 2.96 (m, 2H), 2.67 (m, 2H), 1.52 (s, 3H), 1.50 (s, 6H), 1.48-1.42 (m, 2H).

Table 1 lists examples that were prepared according to the procedures as described in Examples 1-4 by using the corresponding intermediates and reagents under appropriate conditions that could be accomplished by the skilled persons.

| Ex- | | | LC/ | |
|---|---|---|---|---|
| am- | | | MS | |
| ple | | | [M + | |
| # | Structure | Chemical Name | H]⁺ | ¹H NMR |
| 1 | 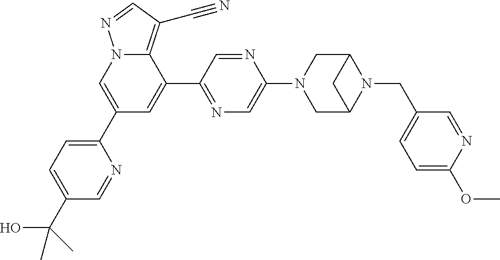 [SMILES: COc1ccc(CN2C3CC2CN(c2cnc(-c4cc(-c5ccc(C(C)(C)O)cn5)cn5ncc(C#N)c45)cn2)C3)cn1] | 6-(5-(2-hydroxy- propan-2-yl)pyri- din-2-yl)-4-(5-(6- ((6-methoxypyridin- 3-yl)methyl)-3,6- diazabi- cyclo[3.1.1]heptan- 3-yl)pyrazin-2- yl)pyrazolo[1,5- a]pyridine-3-carbo- nitrile | 574.4 | ¹H NMR (400 MHz, DMSO-d6) δ 9.56 (s, 1H), 8.82 (s, 1H), 8.76 (s, 1H), 8.73 (s, 1H), 8.47 (s, 1H), 8.32 (s, 1H), 8.19 (d, J = 8.0 Hz, 1H), 8.09 (s, 1H), 7.99 (d, J = 8.2 Hz, 1H), 7.69 (d, J = 8.3 Hz, 1H), 6.76 (d, J = 7.9 Hz, 1H), 5.29 (s, 1H), 3.81 (s, 2H), 3.81 (s, 3H), 3.74-3.58 (m, 4H), 3.54 (s, 2H), 2.55 (s, 1H), 1.63 (d, J = 9.3 Hz, 1H), 1.50 (s, 6H). |
| 2 | 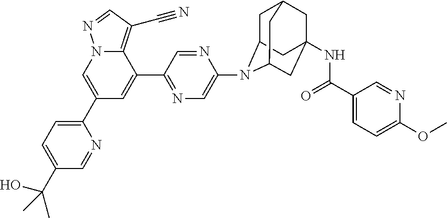 [SMILES: COc1ccc(C(=O)NC23CC4CC(C2)N(c2cnc(-c5cc(-c6ccc(C(C)(C)O)cn6)cn6ncc(C#N)c56)cn2)C(C4)C3)cn1] | N-((1R,3S,5s,7s)-2- (5-(3-cyano-6-(5-(2- hydroxypropan-2- yl)pyridin-2- yl)pyrazolo[1,5-a] pyridin-4-yl)pyra- zin-2-yl)-2-aza- adamantan-5-yl)-6- methoxy- nicotinamide | 642.4 | ¹H NMR (400 MHz, DMSO-d6) δ 9.55 (s, 1H), 8.82 (s, 1H), 8.75 (s, 1H), 8.65 (s, 1H), 8.59 (s, 1H), 8.46 (d, J = 8.8 Hz, 2H), 8.19 (d, J = 8.2 Hz, 1H), 8.06 (d, J = 8.7 Hz, 1H), 7.98 (d, J = 8.2 Hz, 1H), 7.86 (s, 1H), 6.84 (d, J = 8.7 Hz, 1H), 5.29 (s, 1H), 4.93 (s, 2H), 3.88 (s, 3H), 2.35-2.24 (m, 4H), 2.21 (s, 1H), 2.15 (m, 2H), 1.84-1.75 (m, 4H), |
| | | | | 1.50 (s, 6H). |
| 3 | 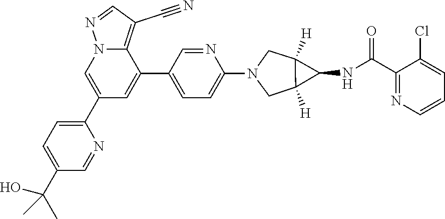 [SMILES: CC(C)(O)c1ccc(-c2cc(-c3ccc(N4C[C@@H]5[C@H](C4)[C@@H]5NC(=O)c4ncccc4Cl)nc3)c3c(C#N)cnn3c2)nc1] | 3-chloro-N- ((1R,5S,6r)-3- (5-(3-cyano-6-(5-(2- hydroxypropan-2- yl)pyridin-2- yl)pyrazolo[1,5- a]pyridin-4-yl)pyri- din-2-yl)-3-aza- bicyclo [3.1.0]hexan- 6-yl)picolinamide | 591.3 | ¹H NMR (400 MHz, DMSO-d6) δ 9.51 (s, 1H), 8.81 (s, 1H), 8.72 (s, 1H), 8.47 (s, 1H), 8.40 (d, J = 4.6 Hz, 1H), 8.33 (s, 1H), 8.16 (d, J = 8.2 Hz, 1H), 8.09 (s, 1H), 7.97 (d, J = 8.3 Hz, 1H), 7.89 (d, J = 8.1 Hz, 1H), 7.77 (d, J = 8.7 Hz, 1H), 7.42 (dd, J = 8.2, 4.6 Hz, 1H), 6.50 (d, J = 8.7 Hz, 1H), 5.29 (s, 1H), 3.81-3.58 (m, 4H), 2.96 (t, J = 7.0 Hz, 1H), |
| | | | | 2.11 (d, J = 7.0 Hz, 2H), |
| | | | | 1.49 (s, 6H). |
| 4 | 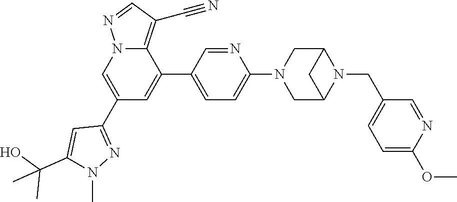 [SMILES: COc1ccc(CN2C3CC2CN(c2ccc(-c4cc(-c5cc(C(C)(C)O)n(C)n5)cn5ncc(C#N)c45)cn2)C3)cn1] | 6-(5-(2-hydroxy- propan-2-yl)- 1-methyl-1H-py- razol-3-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3-carbo- nitrile | 576.6 | ¹H NMR (400 MHz, CDCl₃) δ 8.82 (s, 1H), 8.39 (s, 1H), 8.28 (s, 1H), 8.11 (s, 1H), 7.81 (d, J = 8.7 Hz, 1H), 7.76 (s, 1H), 7.65 (s, 1H), 6.75-6.66 (m, 2H), 6.40 (s, 1H), 4.14 (s, 3H), 3.92 (s, 3H), 3.88- 3.75 (m, 4H), 3.68-3.56 (m, 4H), 2.71 (s, 1H), 1.70 (s, 6H), 1.66 (d, 1H). |
| 5 | 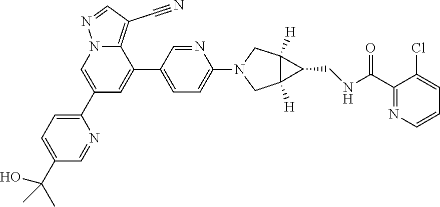 [SMILES: CC(C)(O)c1ccc(-c2cc(-c3ccc(N4C[C@@H]5[C@@H](CNC(=O)c6ncccc6Cl)[C@@H]5C4)nc3)c3c(C#N)cnn3c2)nc1] | 3-chloro-N- (((1R,5S,6s)-3-(5-(3- cyano-6-(5-(2- hydroxypropan-2- yl)pyridin-2- yl)pyrazolo[1,5- a]pyridin-4-yl)pyri- din-2-yl)-3-aza- bicyclo[3.1.0]hexan- 6-yl)meth- yl)picolinamide | 605.4 | ¹H NMR (400 MHz, DMSO-d6) δ 9.51 (s, 1H), 8.80 (s, 1H), 8.76 (t, J = 5.5 Hz, 1H), 8.73 (s, 1H), 8.55 (d, J = 4.5 Hz, 1H), 8.35 (s, 1H), 8.16 (d, J = 8.3 Hz, 1H), 8.13 (s, 1H), 8.03 (d, J = 8.2 Hz, 1H), 7.96 (d, J = 8.3 Hz, 1H), 7.79 (d, J = 8.7 Hz, 1H), 7.53 (dd, J = 8.1, 4.6 Hz, 1H), 6.59 (d, J = 8.7 Hz, 1H), 5.29 (s, 1H), 3.74 (d, |
| | | | | J = 10.5 Hz, 2H), 3.45 (d, |
| | | | | J = 8.9 Hz, 2H), 3.27 (t, J = |
| | | | | 5.4 Hz, 2H), 1.75 (s, |
| | | | | 2H), 1.49 (s, 6H), 0.93 (s, |
| | | | | 1H). |
| 6 | 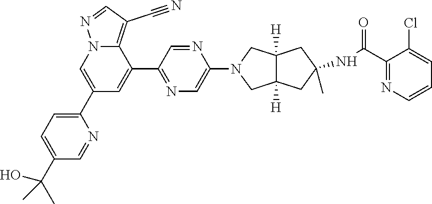 [SMILES: CC(C)(O)c1ccc(-c2cc(-c3cnc(N4C[C@@H]5C[C@@](C)(NC(=O)c6ncccc6Cl)C[C@@H]5C4)cn3)c3c(C#N)cnn3c2)nc1] | 3-chloro-N- ((3aR,5s,6aS)-2-(5- (3-cyano-6-(5-(2- hydroxypropan-2- yl)pyridin-2- yl)pyrazolo[1,5-a] pyridin-4-yl)pyrazin- 2-yl)-5-methyl- octahydrocyclopen- ta[c]pyrrol-5- yl)picolinamide | 634.4 | ¹H NMR (400 MHz, DMSO-d6) δ 9.55 (s, 1H), 8.82 (s, 1H), 8.74 (s, 1H), 8.62 (d, J = 8.2 Hz, 1H), 8.54-8.47 (m, 1H), 8.42 (s, 1H), 8.29 (s, 1H), 8.16 (t, J = 12.8 Hz, 2H), 8.03- 7.92 (m, 2H), 7.48 (m, 1H), 5.30 (s, 1H), 3.75- 3.61 (m, 2H), 3.55 (d, J = 10.7 Hz, 2H), 2.96 (m, 2H), 2.67 (m, 2H), 1.52 (s, 3H), 1.50 (s, 6H), 1.48- |
| | | | | 1.42 (m, 2H). |
| 7 | 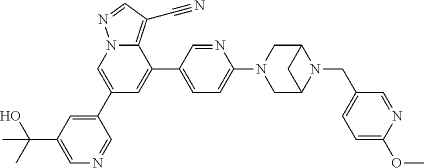 [SMILES: COc1ccc(CN2C3CC2CN(c2ccc(-c4cc(-c5cncc(C(C)(C)O)c5)cn5ncc(C#N)c45)cn2)C3)cn1] | 6-(5-(2-hydroxy- propan-2-yl)pyridin- 3-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3-carbo- nitrile | 573.4 | ¹H NMR (400 MHz, CDCl₃) δ 8.79 (s, 3H), 8.48 (s, 1H), 8.36 (s, 1H), 8.12 (s, 2H), 7.85 (d, J = 8.7 Hz, 1H), 7.65 (s, 1H), 7.56 (s, 1H), 6.73 (d, J = 8.6 Hz, 2H), 3.93 (s, 3H), 3.89-3.75 (m, 4H), 3.69- 3.56 (m, 4H), 2.73 (s, 1H), 1.70 (s, 6H), 1.67 (d, 1H). |
| 8 | 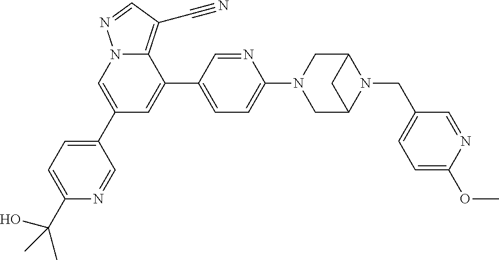 [SMILES: COc1ccc(CN2C3CC2CN(c2ccc(-c4cc(-c5ccc(C(C)(C)O)nc5)cn5ncc(C#N)c45)cn2)C3)cn1] | 6-(6-(2-hydroxy- propan-2-yl)pyridin- 3-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 573.4 | ¹H NMR (400 MHz, CDCl₃) δ 8.81 (s, 1H), 8.77 (s, 1H), 8.47 (s, 1H), 8.36 (s, 1H), 8.11 (s, 1H), 7.95 (d, J = 8.2 Hz, 1H), 7.85 (d, J = 8.7 Hz, 1H), 7.65 (d, J = 8.0 Hz, 1H), 7.57 (d, J = 8.2 Hz, 1H), 7.54 (s, 1H), 6.72 (d, J = 8.6 Hz, 2H), 3.93 (s, 3H), 3.89-3.76 (m, 4H), 3.68- 3.56 (m, 4H), 2.72 (s, 1H), 1.67 (d, J = 4.9 Hz, 1H), 1.62 (s, 6H). |
| 9 | 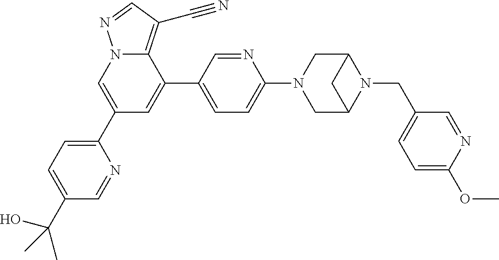 [SMILES: COc1ccc(CN2C3CC2CN(c2ccc(-c4cc(-c5ccc(C(C)(C)O)cn5)cn5ncc(C#N)c45)cn2)C3)cn1] | 6-(5-(2-hydroxy- propan-2-yl)pyridin- 2-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 573.4 | ¹H NMR (400 MHz, DMSO-d6) δ 9.53 (s, 1H), 8.78 (d, J = 25.1 Hz, 2H), 8.48 (s, 1H), 8.17 (d, J = 7.9 Hz, 2H), 8.07 (s, 1H), 7.94 (m, 2H), 7.67 (d, J = 8.5 Hz, 1H), 6.79 (m, 2H), 5.30 (s, 1H), 3.81 (s, 3H), 3.74 (d, J = 12.3 Hz, 2H), 3.67 (d, J = 5.9 Hz, 2H), 3.56 (d, J = 12.1 Hz, 2H), 3.50 (s, 2H), 2.5 (m, 1H), 1.59 (d, J = 8.5 Hz, 1H), 1.50 (s, 6H). |
| 10 | 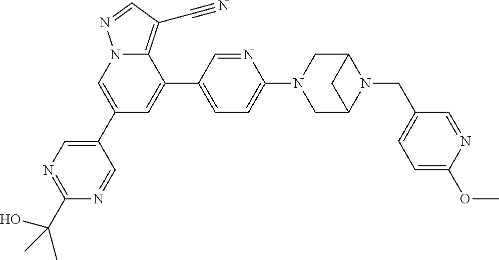 [SMILES: COc1ccc(CN2C3CC2CN(c2ccc(-c4cc(-c5cnc(C(C)(C)O)nc5)cn5ncc(C#N)c45)cn2)C3)cn1] | 6-(2-(2-hydroxy- propan-2- yl)pyrimidin-5-yl)- 4-(6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 574.4 | ¹H NMR (400 MHz, DMSO-d6) δ 9.52 (s, 1H), 9.32 (s, 2H), 8.78 (s, 1H), 8.52 (s, 1H), 8.07 (s, 1H), 8.01-7.91 (m, 2H), 7.67 (s, 1H), 6.83 (d, J = 8.8 Hz, 1H), 5.74 (s, 1H), 5.15 (s, 1H), 3.81 (s, 3H), 3.70 (m, 4H), 3.50 (m, 4H), 2.51 (m, 1H), 1.59 (s, 1H), 1.54 (s, 6H). |
| 11 | 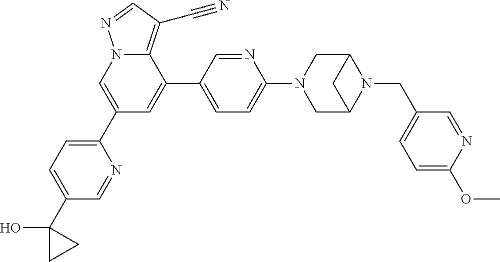 [SMILES: COc1ccc(CN2C3CC2CN(c2ccc(-c4cc(-c5ccc(C6(O)CC6)cn5)cn5ncc(C#N)c45)cn2)C3)cn1] | 6-(5-(1-hydroxy- cyclopropyl)pyri- din-2-yl)-4-(6-(6- ((6-methoxypyri- din-3-yl)methyl)- 3,6-diazabi- cyclo[3.1.1]heptan- 3-yl)pyridin-3- yl)pyrazolo[1,5- a]pyridine-3- carbonitrile | 571.3 | ¹H NMR (400 MHz, DMSO-d6) δ 9.53 (s, 1H), 8.75 (s, 1H), 8.61 (s, 1H), 8.48 (s, 1H), 8.17 (d, J = 11.8 Hz, 2H), 8.07 (s, 1H), 7.91 (d, J = 8.4 Hz, 1H), 7.72 (d, J = 8.1 Hz, 1H), 7.67 (d, J = 8.3 Hz, 1H), 6.82 (d, J = 8.7 Hz, 1H), 6.76 (d, J = 8.5 Hz, 1H), 6.19 (s, 1H), 3.81 (s, 3H), 3.74 (d, J = 12.0 Hz, 2H), 3.68 (d, J = 4.9 Hz, 2H), 3.56 (d, J = 11.6 Hz, 2H), 3.50 (s, 2H), 2.51 (s, 1H), 1.59 (d, J = 8.2 Hz, 1H), |
| | | | | 1.19 (m, 2H), 1.09 (m, |
| | | | | 2H). |
| 12 | 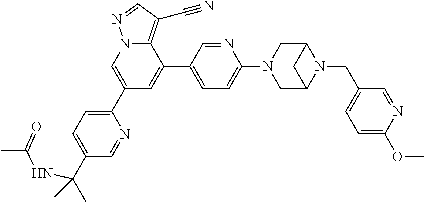 [SMILES: COc1ccc(CN2C3CC2CN(c2ccc(-c4cc(-c5ccc(C(C)(C)NC(C)=O)cn5)cn5ncc(C#N)c45)cn2)C3)cn1] | N-(2-(6-(3-cyano-4- (6-(6-((6- methoxypyridin- 3-yl)methyl)-3,6- diazabi- cyclo[3.1.1]heptan- 3-yl)pyridin-3- yl)pyrazolo[1,5-a] pyridin-6-yl)pyri- din-3-yl)propan-2- yl)acetamide | 614.3 | ¹H NMR (400 MHz, DMSO-d6) δ 9.53 (s, 1H), 8.75 (s, 1H), 8.65 (s, 1H), 8.48 (s, 1H), 8.23 (s, 1H), 8.17 (s, 1H), 8.14 (d, J = 8.3 Hz, 1H), 8.07 (s, 1H), 7.91 (d, J = 8.7 Hz, 1H), 7.81 (d, J = 8.3 Hz, 1H), 7.68 (d, J = 8.2 Hz, 1H), 6.82 (d, J = 8.8 Hz, 1H), 6.76 (d, J = 8.4 Hz, 1H), 3.82 (s, 3H), 3.75 (d, J = 12.2 Hz, 2H), 3.68 (s, 2H), |
| | | | | 3.56 (d, J = 10.4 Hz, 2H), |
| | | | | 3.51 (s, 2H), 2.51 (s, 1H), |
| | | | | 1.85 (s, 3H), 1.60 (s, 1H), |
| | | | | 1.59 (s, 6H). |
| 13 | 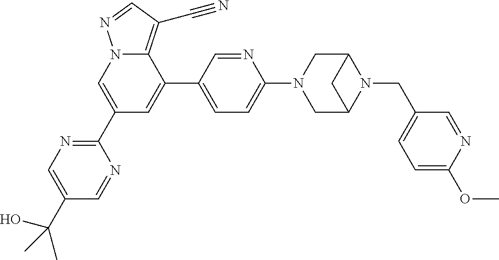 [SMILES: COc1ccc(CN2C3CC2CN(c2ccc(-c4cc(-c5ncc(C(C)(C)O)cn5)cn5ncc(C#N)c45)cn2)C3)cn1] | 6-(5-(2-hydroxy- propan-2-yl)pyrimi- din-2-yl)-4- (6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 574.4 | ¹H NMR (400 MHz, CDCl₃) δ 9.62 (d, J = 11.2 Hz, 1H), 8.94 (s, 2H), 8.50 (s, 1H), 8.42 (s, 1H), 8.36 (s, 1H), 8.12 (s, 1H), 7.86 (d, J = 8.7 Hz, 1H), 7.72 (s, 1H), 6.72 (t, J = 7.7 Hz, 2H), 3.92 (s, 3H), 3.90- 3.83 (m, 4H), 3.68-3.61 (m, 4H), 2.80 (s, 1H), 1.69 (s, 6H), 1.62 (d, 1H). |
| 14 | 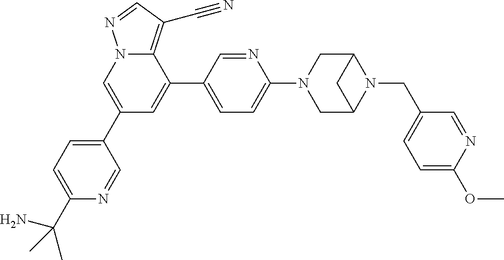 [SMILES: COc1ccc(CN2C3CC2CN(c2ccc(-c4cc(-c5ccc(C(C)(C)N)nc5)cn5ncc(C#N)c45)cn2)C3)cn1] | 6-(6-(2-amino- propan-2-yl)pyri- din-3-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 572.6 | ¹H NMR (400 MHz, CDCl₃) δ 8.86 (d, J = 10.0 Hz, 2H), 8.41 (s, 1H), 8.33 (s, 1H), 8.04 (d, J = 11.3 Hz, 2H), 7.79 (m, 1H), 7.67 (d, J = 8.2 Hz, 1H), 7.61-7.50 (m, 2H), 6.71 (d, J = 8.3 Hz, 2H), 3.88 (s, 4H), 3.84 (s, 3H), 3.62 (s, 4H), 2.97 (d, J = 7.3 Hz, 1H), 1.75 (d, J = 22.9 Hz, 6H), 1.68 (d, J = 8.9 Hz, 1H). |
| 15 | 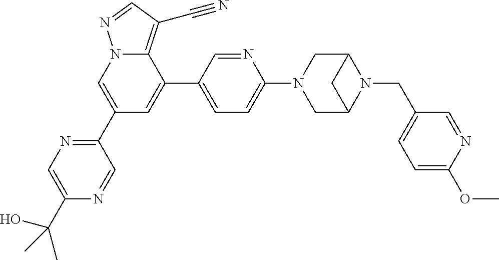 [SMILES: COc1ccc(CN2C3CC2CN(c2ccc(-c4cc(-c5cnc(C(C)(C)O)cn5)cn5ncc(C#N)c45)cn2)C3)cn1] | 6-(5-(2-hydroxy- propan-2-yl)py- razin-2-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 574.4 | ¹H NMR (400 MHz, DMSO-d6) δ 9.65 (s, 1H), 9.36 (s, 1H), 8.99 (s, 1H), 8.79 (s, 1H), 8.48 (s, 1H), 8.17 (s, 1H), 8.07 (s, 1H), 7.91 (d, J = 8.7 Hz, 1H), 7.67 (d, J = 8.3 Hz, 1H), 6.82 (d, J = 8.9 Hz, 1H), 6.76 (d, J = 8.5 Hz, 1H), 5.55 (s, 1H), 3.81 (s, 3H), 3.75 (d, J = 12.0 Hz, 2H), 3.67 (d, J = 4.7 Hz, 2H), 3.56 (d, J = 11.6 Hz, 2H), 3.50 (s, 2H), 2.51 (s, 1H), 1.59 (d, J = 8.2 Hz, 1H), 1.51 (s, 6H). |
| 16 | 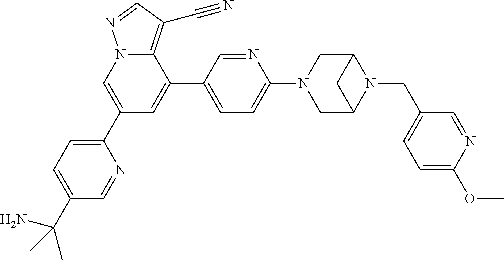 [SMILES: COc1ccc(CN2C3CC2CN(c2ccc(-c4cc(-c5ccc(C(C)(C)N)cn5)cn5ncc(C#N)c45)cn2)C3)cn1] | 6-(5-(2-amino- propan-2-yl)pyridin- 2-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 572.3 | ¹H NMR (400 MHz, DMSO-d6) δ 9.53 (s, 1H), 8.88 (s, 1H), 8.75 (s, 1H), 8.48 (d, J = 2.5 Hz, 1H), 8.21-8.13 (m, 2H), 8.10- 8.03 (m, 2H), 7.91 (dd, J = 8.9, 2.5 Hz, 1H), 7.71- 7.65 (m, 1H), 6.82 (d, J = 8.8 Hz, 1H), 6.76 (d, J = 8.3 Hz, 1H), 3.81 (s, 3H), 3.74 (d, J = 12.4 Hz, 2H), 3.67 (d, J = 5.9 Hz, 2H), 3.56 (d, J = 12.2 Hz, 2H), 3.50 (s, 2H), 2.52 (s, 1H), 1.58 (d, J = 8.4 Hz, 1H), 1.43 (s, 6H). |
| 17 | 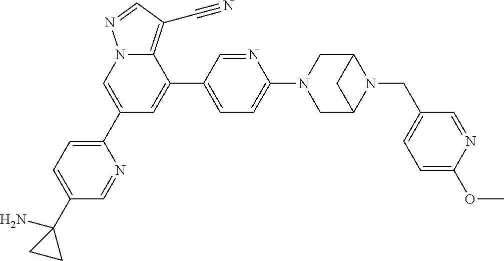 [SMILES: COc1ccc(CN2C3CC2CN(c2ccc(-c4cc(-c5ccc(C6(N)CC6)cn5)cn5ncc(C#N)c45)cn2)C3)cn1] | 6-(5-(1-amino-cy- clopropyl)pyridin- 2-yl)-4-(6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 570.3 | ¹H NMR (400 MHz, DMSO-d6) δ 9.52 (s, 1H), 8.74 (s, 1H), 8.66 (d, J = 1.6 Hz, 1H), 8.47 (d, J = 1.9 Hz, 1H), 8.17 (s, 1H), 8.14 (d, J = 8.4 Hz, 1H), 8.06 (s, 1H), 7.90 (dd, J = 8.7, 2.2 Hz, 1H), 7.80 (dd, J = 8.4, 2.2 Hz, 1H), 7.67 (dd, J = 8.5, 2.0 Hz, 1H), 6.81 (d, J = 8.8 Hz, 1H), 6.76 (d, J = 8.5 Hz, 1H), 3.81 (s, 3H), 3.74 (d, J = 12.1 Hz, 2H), 3.67 (d, J = 5.6 Hz, 2H), 3.55 (d, J = 11.4 Hz, 2H), 3.50 (s, 2H), |
| | | | | 2.71-2.53 (m, 2H), 2.53- |
| | | | | 2.50 (m, 1H), 1.59 (d, J = |
| | | | | 8.4 Hz, 1H), 1.04 (s, 4H). |
| 18 | 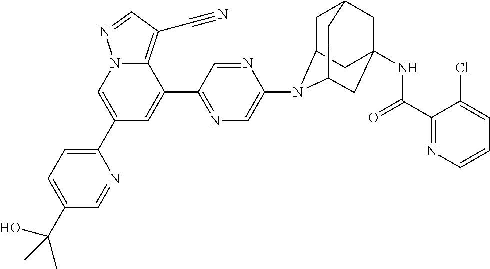 [SMILES: CC(C)(O)c1ccc(-c2cc(-c3cnc(N4C5CC6CC4CC(NC(=O)c4ncccc4Cl)(C6)C5)cn3)c3c(C#N)cnn3c2)nc1] | 3-chloro-N- ((1R,3S,5s,7s)-2- (5-(3-cyano-6-(5- (2-hydroxypropan- 2-yl)pyridin-2- yl)pyrazolo[1,5- a]pyridin-4- yl)pyrazin-2-yl)-2- azaadamantan- 5-yl)picolinamide | 646.3 | ¹H NMR (400 MHz, CD₃OD) δ 9.29 (s, 1H), 8.81 (s, 1H), 8.54 (s, 1H), 8.46 (d, J = 7.7 Hz, 2H), 8.34 (s, 1H), 8.28 (s, 1H), 7.99 (dd, J = 21.1, 8.2 Hz, 2H), 7.92 (d, J = 8.1 Hz, 1H), 7.49-7.40 (m, 1H), 4.95 (s, 2H), 2.37 (m, 3H), 2.28 (m, 4H), 1.95 (m, 2H), 1.87 (m, 2H), 1.60 (s, 6H). |
| 19 | 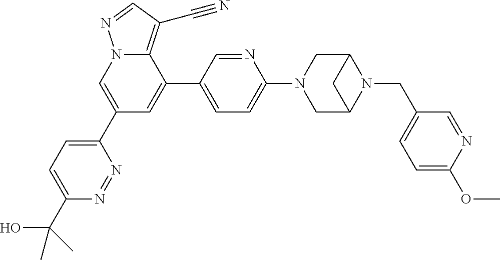 [SMILES: COc1ccc(CN2C3CC2CN(c2ccc(-c4cc(-c5ccc(C(C)(C)O)nn5)cn5ncc(C#N)c45)cn2)C3)cn1] | 6-(6-(2-hydroxy- propan-2-yl)pyrida- zin-3-yl)-4- (6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 574.4 | ¹H NMR (400 MHz, DMSO-d6) δ 9.67 (s, 1H), 8.80 (s, 1H), 8.50 (d, J = 8.7 Hz, 2H), 8.27 (s, 1H), 8.07 (s, 1H), 8.02 (d, J = 9.0 Hz, 1H), 7.93 (d, J = 8.8 Hz, 1H), 7.67 (d, J = 8.1 Hz, 1H), 6.83 (d, J = 8.7 Hz, 1H), 6.76 (d, J = 8.3 Hz, 1H), 5.57 (s, 1H), 3.81 (s, 3H), 3.75 (d, J = 11.7 Hz, 2H), 3.67 (d, J = 5.4 Hz, 2H), 3.56 (d, J = 9.0 Hz, 2H), 3.51 (s, 2H), 2.50 (s, 1H), 1.60 (s, 1H), 1.58 (s, 6H). |
| 20 | 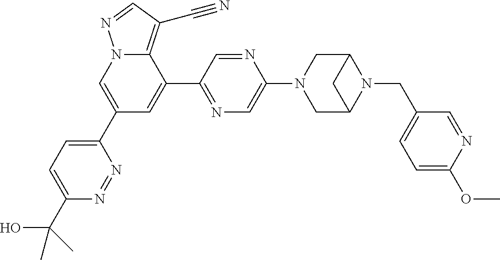 [SMILES: COc1ccc(CN2C3CC2CN(c2cnc(-c4cc(-c5ccc(C(C)(C)O)nn5)cn5ncc(C#N)c45)cn2)C3)cn1] | 6-(6-(2-hydroxy- propan-2- yl)pyridazin-3-yl)- 4-(5-(6-((6- methoxypyridin-3- yl)methyl)-3,6- diazabi- cyclo[3.1.1]heptan- 3-yl)pyrazin-2- yl)pyrazolo[1,5- a]pyridine-3- carbonitrile | 575.4 | ¹H NMR (400 MHz, DMSO-d6) δ 9.70 (s, 1H), 8.81 (s, 1H), 8.76 (s, 1H), 8.57 (s, 1H), 8.51 (d, J = 8.8 Hz, 1H), 8.33 (s, 1H), 8.09 (s, 1H), 8.03 (d, J = 8.9 Hz, 1H), 7.69 (d, J = 8.4 Hz, 1H), 6.76 (d, J = 8.3 Hz, 1H), 5.57 (s, 1H), 3.84 (s, 2H), 3.81 (s, 3H), 3.69 (d, J = 4.7 Hz, 2H), 3.64 (d, J = 12.2 Hz, 2H), 3.54 (s, 2H), 2.55 (d, J = 5.8 Hz, 1H), 1.63 (d, J = 8.7 Hz, 1H), 1.59 (s, 6H). |
| 21 | 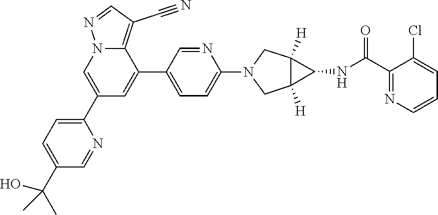 [SMILES: CC(C)(O)c1ccc(-c2cc(-c3ccc(N4C[C@@H]5[C@H](C4)[C@H]5NC(=O)c4ncccc4Cl)nc3)c3c(C#N)cnn3c2)nc1] | 3-chloro-N- ((1R,5S,6s)-3-(5- (3-cyano-6-(5-(2- hydroxypropan-2- yl)pyridin-2- yl)pyrazolo[1,5- a]pyridin-4-yl)pyri- din-2-yl)-3-aza- bicy- clo[3.1.0]hexan- 6-yl)picolinamide | 591.3 | ¹H NMR (400 MHz, DMSO-d6) δ 9.48 (s, 1H), 8.80 (d, J = 4.0 Hz, 1H), 8.77 (s, 1H), 8.70 (s, 1H), 8.50 (d, J = 4.6 Hz, 1H), 8.35 (s, 1H), 8.12 (d, J = 9.8 Hz, 2H), 7.99 (d, J = 8.2 Hz, 1H), 7.93 (d, J = 8.3 Hz, 1H), 7.79 (d, J = 8.7 Hz, 1H), 7.50 (dd, J = 8.1, 4.7 Hz, 1H), 6.61 (d, J = 8.7 Hz, 1H), 5.24 (s, 1H), 3.82 (d, J = 10.7 Hz, 2H), 3.49 (d, J = 10.1 Hz, |
| | | | | 2H), 2.62 (s, 1H), 1.96 (s, |
| | | | | 2H), 1.45 (s, 6H). |
| 22 | 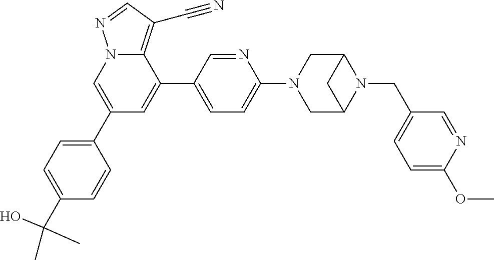 [SMILES: COc1ccc(CN2C3CC2CN(c2ccc(-c4cc(-c5ccc(C(C)(C)O)cc5)cn5ncc(C#N)c45)cn2)C3)cn1] | 6-(4-(2-hydroxy- propan-2-yl)phe- nyl)-4-(6-(6-((6- methoxypyridin-3- yl)methyl)-3,6- diazabicyclo [3.1.1]heptan- 3-yl)pyridin-3- yl)pyrazolo[1,5- a]pyridine-3- carbonitrile | 572.3 | ¹H NMR (400 MHz, DMSO-d6) δ 9.25 (s, 1H), 8.72 (s, 1H), 8.50 (s, 1H), 8.07 (s, 1H), 7.93 (dd, J = 9.0, 1.9 Hz, 1H), 7.84 (s, 1H), 7.82 (s, 2H), 7.68 (dd, J = 8.3, 1.5 Hz, 1H), 7.61 (s, 1H), 7.59 (s, 1H), 6.81 (d, J = 9.0 Hz, 1H), 6.76 (d, J = 8.3 Hz, 1H), 5.09 (s, 1H), 3.82 (s, 3H), 3.74 (d, J = 12.4 Hz, 2H), 3.68 (d, J = 5.6 Hz, 2H), 3.56 (d, J = 12.2 Hz, 2H), 3.50 (s, 2H), 2.57-2.51 (m, 1H), 1.59 (d, J = 8.3 Hz, |
| | | | | 1H), 1.46 (s, 6H). |
| 23 | 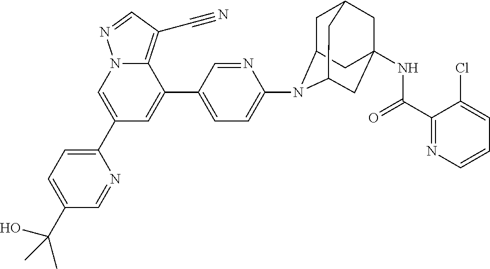 [SMILES: CC(C)(O)c1ccc(-c2cc(-c3ccc(N4C5CC6CC4CC(NC(=O)c4ncccc4Cl)(C6)C5)nc3)c3c(C#N)cnn3c2)nc1] | 3-chloro-N- ((1R,3S,5s,7s)-2- (5-(3-cyano-6-(5- (2-hydroxypropan- 2-yl)pyridin-2- yl)pyrazolo[1,5- a]pyridin-4- yl)pyridin-2-yl)- 2-azaadamantan- 5-yl)picolinamide | 645.4 | ¹H NMR (400 MHz, CD₃OD) δ 9.28 (d, J = 1.1 Hz, 1H), 8.81 (d, J = 1.8 Hz, 1H), 8.57-8.42 (m, 2H), 8.36 (d, J = 2.2 Hz, 1H), 8.07-7.98 (m, 2H), 7.97-7.90 (m, 2H), 7.80 (dd, J = 8.9, 2.3 Hz, 1H), 7.46 (dd, J = 8.1, 4.7 Hz, 1H), 6.94 (d, J = 8.9 Hz, 1H), 4.84 (s, 2H), 2.43- 2.28 (m, 5H), 2.23 (m, 2H), 1.94 (m, 2H), 1.83 (m, 2H), 1.60 (s, 6H). |
| 24 | 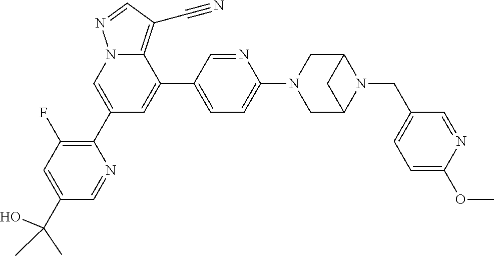 [SMILES: COc1ccc(CN2C3CC2CN(c2ccc(-c4cc(-c5ncc(C(C)(C)O)cc5F)cn5ncc(C#N)c45)cn2)C3)cn1] | 6-(3-fluoro-5-(2- hydroxypropan-2- yl)pyridin-2-yl)- 4-(6-(6- ((6-methoxypyri- din-3-yl)meth- yl)-3,6-diazabi- cyclo[3.1.1]heptan- 3-yl)pyridin-3- yl)pyrazolo[1,5- a]pyridine-3- carbonitrile | 591.3 | ¹H NMR (400 MHz, CDCl₃) δ 9.27 (s, 1H), 8.64 (s, 1H), 8.49 (s, 1H), 8.35 (s, 1H), 8.17 (s, 1H), 8.11 (s, 1H), 7.85 (d, J = 8.9 Hz, 1H), 7.73 (d, J = 12.5 Hz, 1H), 7.63 (d, J = 8.9 Hz, 1H), 6.72 (d, J = 8.3 Hz, 2H), 3.92 (s, 3H), 3.85 (m, 2H), 3.78 (m, 2H), 3.59 (s, 4H), 2.69 (d, J = 7.3 Hz, 1H), 1.73 (s, 1H), 1.68 (s, 6H). |
| 25 | 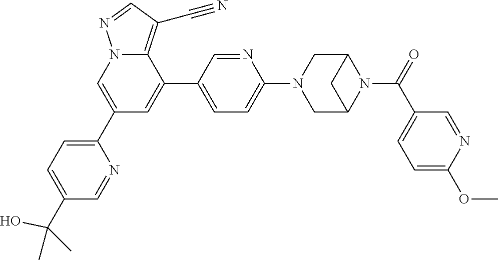 [SMILES: COc1ccc(C(=O)N2C3CC2CN(c2ccc(-c4cc(-c5ccc(C(C)(C)O)cn5)cn5ncc(C#N)c45)cn2)C3)cn1] | 6-(5-(2-hydroxy- propan-2-yl)pyridin- 2-yl)-4-(6-(6-(6- methoxynicotinoyl)- 3,6-diazabi- cyclo[3.1.1]heptan- 3-yl)pyridin-3- yl)pyrazolo[1,5- a]pyridine-3- carbonitrile | 587.2 | ¹H NMR (400 MHz, DMSO-d6) δ 9.52 (s, 1H), 8.80 (d, J = 1.5 Hz, 1H), 8.74 (s, 1H), 8.54 (d, J = 1.7 Hz, 1H), 8.40 (d, J = 1.7 Hz, 1H), 8.16 (d, J = 7.7 Hz, 2H), 8.00-7.94 (m, 2H), 7.86 (dd, J = 8.7, 2.2 Hz, 1H), 6.86 (d, J = 8.7 Hz, 1H), 6.73 (d, J = 8.8 Hz, 1H), 5.29 (s, 1H), 4.93 (s, 1H), 4.61 (s, 1H), 4.18 (d, J = 10.7 Hz, 1H), 3.91 (s, 3H), 3.66 (d, J = 11.6 Hz, 2H), 3.52 (d, J = 10.5 Hz, 1H), 2.88-2.80 |
| | | | | (m, 1H), 1.70 (d, J = 8.7 |
| | | | | Hz, 1H), 1.49 (s, 6H). |
| 26 | 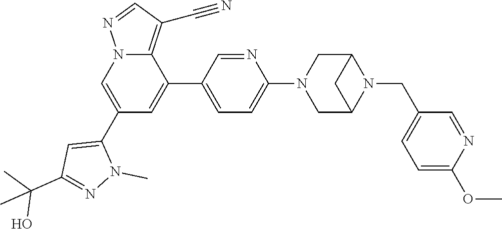 [SMILES: COc1ccc(CN2C3CC2CN(c2ccc(-c4cc(-c5cc(C(C)(C)O)nn5C)cn5ncc(C#N)c45)cn2)C3)cn1] | 6-(3-(2-hydroxy- propan-2-yl)-1- methyl-1H-pyrazol- 5-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 576.5 | ¹H NMR (400 MHz, DMSO-d6) δ 9.14 (s, 1H), 8.77 (s, 1H), 8.47 (s, 1H), 8.06 (s, 1H), 7.90 (d, J = 8.7 Hz, 1H), 7.67 (d, J = 8.4 Hz, 1H), 7.61 (s, 1H), 6.78 (dd, J = 19.0, 8.7 Hz, 2H), 6.60 (s, 1H), 4.90 (s, 1H), 3.90 (s, 3H), 3.81 (s, 3H), 3.73 (d, J = 11.4 Hz, 2H), 3.67 (d, J = 5.1 Hz, 2H), 3.56 (s, 2H), 3.50 (s, 2H), 2.53 (s, 1H), 1.58 (d, J = 8.4 Hz, 1H), 1.45 (s, |
| | | | | 6H). |
| 27 | 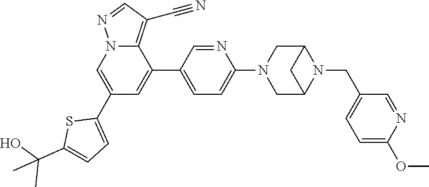 [SMILES: COc1ccc(CN2C3CC2CN(c2ccc(-c4cc(-c5ccc(C(C)(C)O)s5)cn5ncc(C#N)c45)cn2)C3)cn1] | 6-(5-(2-hydroxy- propan-2-yl)thio- phen-2-yl)-4- (6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 578.4 | ¹H NMR (400 MHz, CDCl₃) δ 8.72 (s, 1H), 8.44 (s, 1H), 8.30 (s, 1H), 8.11 (s, 1H), 7.82 (d, J = 8.9 Hz, 1H), 7.63 (d, J = 8.5 Hz, 1H), 7.52 (s, 1H), 7.21 (d, J = 3.7 Hz, 1H), 6.98 (d, J = 3.7 Hz, 1H), 6.72 (d, J = 3.5 Hz, 1H), 6.70 (d, J = 4.3 Hz, 1H), 3.92 (s, 3H), 3.89-3.75 (m, 4H), 3.67-3.55 (m, |
| | | | | 4H), 2.68 (s, 1H), 1.72 (s, |
| | | | | 6H), 1.66 (d, J = 8.7 Hz, |
| | | | | 1H). |
| 28 | 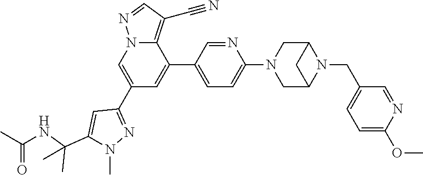 [SMILES: COc1ccc(CN2C3CC2CN(c2ccc(-c4cc(-c5cc(C(C)(C)NC(C)=O)n(C)n5)cn5ncc(C#N)c45)cn2)C3)cn1] | N-(2-(3-(3-cyano-4- (6-(6-((6- methoxypyridin- 3-yl)methyl)-3,6- diazabi- cyclo[3.1.1]heptan- 3-yl)pyridin-3- yl)pyrazolo[1,5- a]pyridin-6-yl)-1- methyl-1H-pyrazol- 5-yl)propan-2- | 617.4 | ¹H NMR (400 MHz, CDCl₃) δ 8.88 (s, 1H), 8.45 (s, 1H), 8.28 (s, 1H), 8.11 (s, 1H), 7.85-7.80 (m, 1H), 7.78 (s, 1H), 7.63 (d, J = 8.6 Hz, 1H), 6.70 (t, J = 8.8 Hz, 2H), 6.49 (s, 1H), 5.73 (s, 1H), 3.98 (s, 3H), 3.92 (s, 3H), 3.81 (m, 4H), 3.60 (m, 4H), 2.69 (d, J = 7.1 Hz, 1H), 2.01 (s, |
| | | yl)acetamide | | 3H), 1.77 (s, 6H), 1.67 (d, |
| | | | | J = 8.4 Hz, 1H). |
| 29 | 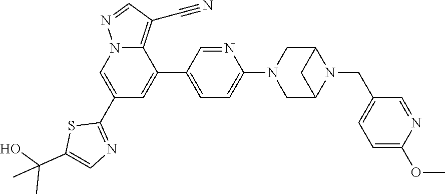 [SMILES: COc1ccc(CN2C3CC2CN(c2ccc(-c4cc(-c5ncc(C(C)(C)O)s5)cn5ncc(C#N)c45)cn2)C3)cn1] | 6-(5-(2-hydroxy- propan-2-yl)thiazol- 2-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 579.3 | ¹H NMR (400 MHz, DMSO-d6) δ 9.39 (d, J = 1.5 Hz, 1H), 8.78 (s, 1H), 8.47 (d, J = 2.5 Hz, 1H), 8.07 (d, J = 1.8 Hz, 1H), 7.90 (dd, J = 8.8, 2.5 Hz, 1H), 7.88 (d, J = 1.5 Hz, 1H), 7.77 (s, 1H), 7.67 (dd, J = 8.5, 2.3 Hz, 1H), 6.82 (d, J = 8.9 Hz, 1H), 6.76 (d, J = 8.5 Hz, 1H), 5.79 (s, 1H), 3.81 (s, 3H), 3.74 |
| | | | | (m, 2H), 3.67 (m, 2H), |
| | | | | 3.61-3.52 (m, 2H), 3.50 |
| | | | | (s, 2H), 2.56-2.50 (m, |
| | | | | 1H), 1.60 (s, 1H), 1.57 (s, |
| | | | | 6H). |
| 30 | 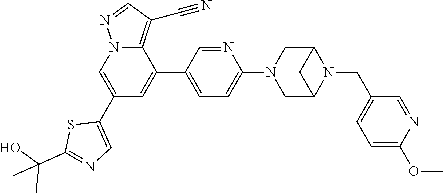 [SMILES: COc1ccc(CN2C3CC2CN(c2ccc(-c4cc(-c5cnc(C(C)(C)O)s5)cn5ncc(C#N)c45)cn2)C3)cn1] | 6-(2-(2-hydroxy- propan-2-yl)thi- azol-5-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 579.3 | ¹H NMR (400 MHz, CDCl₃) δ 8.73 (s, 1H), 8.45 (d, J = 2.1 Hz, 1H), 8.32 (s, 1H), 8.10 (s, 1H), 7.91 (s, 1H), 7.82 (dd, J = 8.8, 2.4 Hz, 1H), 7.63 (d, J = 8.4 Hz, 1H), 7.48 (s, 1H), 6.73 (s, 1H), 6.71 (s, 1H), 3.92 (s, 3H), 3.89- 3.76 (m, 4H), 3.68-3.54 (m, 4H), 2.70 (d, J = 7.3 Hz, 1H), 1.74 (s, 6H), 1.66 |
| | | | | (d, J = 5.2 Hz, 1H). |
| 31 | 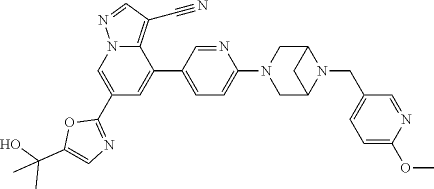 [SMILES: COc1ccc(CN2C3CC2CN(c2ccc(-c4cc(-c5ncc(C(C)(C)O)o5)cn5ncc(C#N)c45)cn2)C3)cn1] | 6-(5-(2-hydroxy- propan-2-yl)oxazol- 2-yl)-4-(6-(6- ((6-methoxypyridin- 3-yl)methyl)-3,6- diazabi- cyclo[3.1.1]heptan- 3-yl)pyridin-3- yl)pyrazolo[1,5- a]pyridine-3- carbonitrile | 563.3 | ¹H NMR (400 MHz, DMSO-d6) δ 9.39 (s, 1H), 8.82 (s, 1H), 8.46 (d, J = 2.1 Hz, 1H), 8.07 (s, 1H), 7.90 (d, J = 8.1 Hz, 1H), 7.86 (s, 1H), 7.68 (d, J = 8.4 Hz, 1H), 7.18 (s, 1H), 6.83 (d, J = 8.8 Hz, 1H), 6.76 (d, J = 8.3 Hz, 1H), 5.47 (s, 1H), 3.82 (s, 3H), 3.78-3.71 (m, 2H), 3.71- 3.64 (m, 2H), 3.63-3.53 |
| | | | | (m, 2H), 3.53-3.44 (m, |
| | | | | 2H), 2.50 (s, 1H), 1.59 (s, |
| | | | | 1H), 1.53 (s, 6H). |
| 32 | 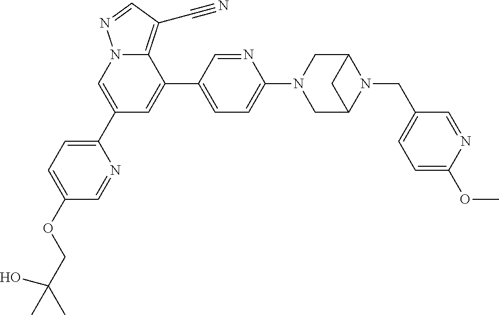 [SMILES: COc1ccc(CN2C3CC2CN(c2ccc(-c4cc(-c5ccc(OCC(C)(C)O)cn5)cn5ncc(C#N)c45)cn2)C3)cn1] | 6-(5-(2-hydroxy-2- methylpro- poxy)pyridin- 2-yl)-4-(6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 603.5 | ¹H NMR (400 MHz, DMSO-d6) δ 9.46 (s, 1H), 8.72 (s, 1H), 8.47 (s, 1H), 8.42 (d, J = 2.2 Hz, 1H), 8.19 (d, J = 8.8 Hz, 1H), 8.14 (s, 1H), 8.06 (s, 1H), 7.89 (d, J = 8.7 Hz, 1H), 7.67 (d, J = 8.5 Hz, 1H), 7.55 (dd, J = 8.8, 2.3 Hz, 1H), 6.81 (d, J = 8.8 Hz, 1H), 6.76 (d, J = 8.5 Hz, 1H), 4.70 (s, 1H), 3.87 (s, 2H), 3.81 (s, 3H), 3.74 (d, J = 12.0 Hz, 2H), 3.67 (d, J = 5.3 Hz, 2H), 3.55 (d, J = 11.5 Hz, 2H), 3.50 (s, 2H), 2.51 (s, 1H), 1.59 (d, J = 8.3 Hz, 1H), 1.22 (s, 6H). |
| 33 | 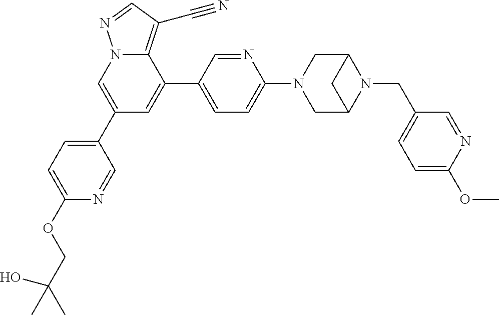 [SMILES: COc1ccc(CN2C3CC2CN(c2ccc(-c4cc(-c5ccc(OCC(C)(C)O)nc5)cn5ncc(C#N)c45)cn2)C3)cn1] | 6-(6-(2-hydroxy-2- methylpro- poxy)pyridin- 3-yl)-4-(6-(6-((6- methoxypyridin-3- yl)methyl)-3,6- diazabi- cyclo[3.1.1]heptan- 3-yl)pyridin-3- yl)pyrazolo[1,5- a]pyridine-3- carbonitrile | 603.5 | ¹H NMR (400 MHz, DMSO-d6) δ 9.17 (s, 1H), 8.70 (s, 1H), 8.46 (d, J = 2.5 Hz, 1H), 8.23 (d, J = 2.7 Hz, 1H), 8.05 (dd, J = 9.7, 2.5 Hz, 2H), 7.89 (dd, J = 8.7, 2.5 Hz, 1H), 7.72 (s, 1H), 7.67 (dd, J = 8.5, 2.3 Hz, 1H), 6.81 (d, J = 8.8 Hz, 1H), 6.76 (d, J = 8.5 Hz, 1H), 6.54 (d, J = 9.4 Hz, 1H), 4.82 (s, 1H), 3.99 (s, 2H), 3.81 (d, J = 1.7 Hz, 3H), 3.73 (d, J = 12.4 Hz, 2H), 3.67 (d, J = 5.9 Hz, 2H), 3.55 (d, J = 12.3 Hz, 2H), 3.49 (s, 2H), 2.53 (s, 1H), 1.58 (d, J = 8.4 Hz, 1H), 1.10 (s, 6H). |
| 34 | 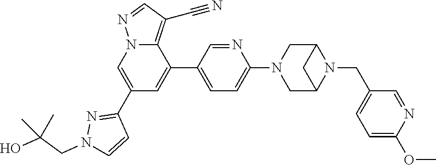 [SMILES: COc1ccc(CN2C3CC2CN(c2ccc(-c4cc(-c5ccn(CC(C)(C)O)n5)cn5ncc(C#N)c45)cn2)C3)cn1] | 6-(1-(2-hydroxy-2- methylpropyl)-1H- pyrazol-3-yl)-4-(6- (6-((6-methoxypyri- din-3-yl)meth- yl)-3,6-diazabi- cyclo[3.1.1]heptan- 3-yl)pyridin-3- yl)pyrazolo[1,5- a]pyridine-3- | 576.4 | ¹H NMR (400 MHz, DMSO-d6) δ 9.28 (s, 1H), 8.70 (s, 1H), 8.44 (d, J = 1.7 Hz, 1H), 8.07 (s, 1H), 7.90 (s, 1H), 7.87 (dd, J = 8.7, 1.9 Hz, 1H), 7.79 (d, J = 1.8 Hz, 1H), 7.68 (d, J = 8.6 Hz, 1H), 7.00 (d, J = 1.9 Hz, 1H), 6.82 (d, J = 8.7 Hz, 1H), 6.76 (d, |
| | | carbonitrile | | J = 8.5 Hz, 1H), 4.72 (s, |
| | | | | 1H), 4.08 (s, 2H), 3.82 (s, |
| | | | | 3H), 3.74 (m, 2H), 3.68 |
| | | | | (m, 2H), 3.55 (m, 2H), |
| | | | | 3.50 (s, 2H), 2.57-2.52 |
| | | | | (m, 1H), 1.59 (d, J = 8.5 |
| | | | | Hz, 1H), 1.10 (s, 6H). |
| 35 | 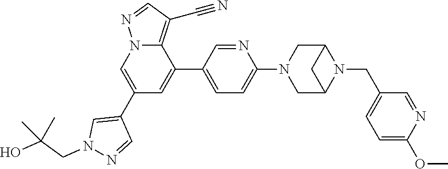 [SMILES: COc1ccc(CN2C3CC2CN(c2ccc(-c4cc(-c5cnn(CC(C)(C)O)c5)cn5ncc(C#N)c45)cn2)C3)cn1] | 6-(1-(2-hydroxy-2- methylpropyl)-1H- pyrazol-4-yl)-4-(6- (6-((6-methoxypyri- din-3-yl)methyl)- 3,6-diazabi- cyclo[3.1.1]heptan- 3-yl)pyridin-3- yl)pyrazolo[1,5- a]pyridine-3- carbonitrile | 576.5 | ¹H NMR (400 MHz, DMSO-d6) δ 9.26 (s, 1H), 8.65 (s, 1H), 8.45 (d, J = 2.2 Hz, 1H), 8.39 (s, 1H), 8.14 (s, 1H), 8.06 (s, 1H), 7.88 (dd, J = 8.7, 2.3 Hz, 1H), 7.82 (s, 1H), 7.67 (dd, J = 8.6, 2.1 Hz, 1H), 6.81 (d, J = 8.8 Hz, 1H), 6.76 (d, J = 8.5 Hz, 1H), 4.75 (s, 1H), 4.03 (s, 2H), 3.82 |
| | | | | (s, 3H), 3.74 (d, J = 11.7 |
| | | | | Hz, 2H), 3.68 (d, J = 5.4 |
| | | | | Hz, 2H), 3.55 (d, J = |
| | | | | 12.3 Hz, 2H), 3.50 (s, 2H), |
| | | | | 2.57-2.51 (m, 1H), |
| | | | | 1.59 (d, J = 8.3 Hz, 1H), |
| | | | | 1.10 (s, 6H). |
| 36 | 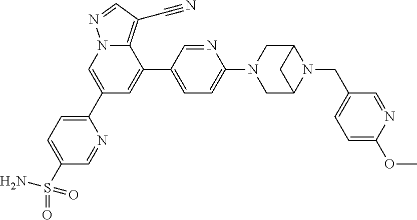 [SMILES: COc1ccc(CN2C3CC2CN(c2ccc(-c4cc(-c5ccc(S(N)(=O)=O)cn5)cn5ncc(C#N)c45)cn2)C3)cn1] | 6-(3-cyano-4-(6-(6- ((6-methoxypyri- din-3-yl)meth yl)-3,6-diazabi- cyclo[3.1.1]heptan- 3-yl)pyridin-3- yl)pyrazolo[1,5-a] pyridin-6-yl)pyri- dine-3-sulfonamide | 594.2 | ¹H NMR (400 MHz, DMSO-d6) δ 9.66 (s, 1H), 9.07 (s, 1H), 8.80 (s, 1H), 8.47 (d, J = 11.7 Hz, 2H), 8.30 (d, J = 8.5 Hz, 1H), 8.21 (s, 1H), 8.06 (s, 1H), 7.91 (d, J = 8.7 Hz, 1H), 7.68 (s, 3H), 6.82 (d, J = 8.9 Hz, 1H), 6.76 (d, J = 8.4 Hz, 1H), 3.81 (s, 3H), 3.74 (d, J = 12.5 Hz, 2H), 3.67 (d, J = 3.9 Hz, 2H), 3.56 (d, J = 11.8 Hz, 2H), 3.50 (s, 2H), 2.51 (s, 1H), |
| | | | | 1.59 (d, J = 8.3 Hz, 1H). |
| 37 | 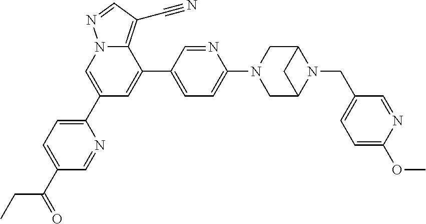 [SMILES: CCC(=O)c1ccc(-c2cc(-c3ccc(N4CC5CC(C4)N5Cc4ccc(OC)nc4)nc3)c3c(C#N)cnn3c2)nc1] | 4-(6-(6-((6- methoxypyridin-3- yl)methyl)-3,6- diazabi- cyclo[3.1.1]heptan- 3-yl)pyridin-3-yl)-6- (5-propionylpyridin- 2-yl)pyrazolo[1,5- a]pyridine-3- carbonitrile | 571.3 | |

### Enzymatic Assay

### RET Kinase Assay

Compounds were tested by Mobility shift assay with ATP concentration at Km. The assay used human RET kinase (Carna 08-159). Test compounds were prepared and diluted in DMSO in 3-fold serial dilutions to 50× of the final testing concentrations. The compounds were then further diluted to 5× by the kinase reaction buffer (50 mM HEPES pH 7.5, 0.0015% Brij-35). The enzymatic reaction for compound testing was performed in a white 384-well polypropylene plate (Corning 3573) with a total reaction volume of 25 μl containing 7 nM RET, 3 μM peptide substrate FAM-P2 (GL Biochem 112394), and 23 μM ATP (Sigma A7699-1G). The assay started with loading RET diluted in kinase reaction buffer to wells, followed by addition of equal volume of 5× compounds for 15-min incubation at the room temperature for pre-treatment. The enzymatic reaction was initiated by addition of mixture of the substrate and ATP prepared in kinase reaction buffer. After incubation at 28° C. for one hour, 25 μl of stopper buffer (a mixture of 100 mM HEPES pH 7.5 buffer, 0.015% Brij-35, 50 mM EDTA and 0.2% of coating reagent 3 (Cliper Lifesciences)). After 30 minutes of incubation at room temperature, the plate was read in a Caliper. Percent of control was calculated as the percentage of compound-treated vs 2% DMSO vehicle-treated. The dose-response curves were generated and the IC₅₀ values were calculated by nonlinear sigmoid curve fitting using XLFit.

The IC₅₀ values (nM) of RET biochemical activity for the examples disclosed herein are listed in Table 2.

### KDR Kinase Assay

Compounds were tested by Mobility shift assay with ATP concentration at Km. The assay used human KDR kinase (Carna 08-191). Test compounds were prepared and diluted in DMSO in 3-fold serial dilutions to 50× of the final testing concentrations. The compounds were then further diluted to 5× by the kinase reaction buffer (50 mM HEPES pH 7.5, 0.0015% Brij-35). The enzymatic reaction for compound testing was performed in a white 384-well polypropylene plate (Corning 3573) with a total reaction volume of 25 μl containing 1.2 nM KDR, 3 μM peptide substrate FAM-P22 (GL Biochem 112393), and 92 μM ATP (Sigma A7699-1G). The assay started with loading RET diluted in kinase reaction buffer to wells, followed by addition of equal volume of 5× compounds for 15-min incubation at the room temperature for pre-treatment. The enzymatic reaction was initiated by addition of mixture of the substrate and ATP prepared in kinase reaction buffer. After incubation at 28° C. for one hour, 25 μl of stopper buffer (a mixture of 100 mM HEPES pH 7.5 buffer, 0.015% Brij-35, 50 mM EDTA and 0.2% of coating reagent 3 (Cliper Lifesciences)). After 30 minutes of incubation at room temperature, the plate was read in a Caliper. Percent of control was calculated as the percentage of compound-treated vs 2% DMSO vehicle-treated. The dose-response curves were generated and the IC₅₀s were calculated by nonlinear sigmoid curve fitting using XLFit.

The IC₅₀ values (nM) of KDR biochemical activity for the examples disclosed herein are listed in Table 2.

### Aurora B Kinase Assay

Compounds were tested by Mobility shift assay with ATP concentration at Km. The assay used human Aurora B kinase (Carna 05-102). Test compounds were prepared and diluted in DMSO in 3-fold serial dilutions to 50× of the final testing concentrations. The compounds were then further diluted to 5× by the kinase reaction buffer (50 mM HEPES pH 7.5, 0.0015% Brij-35). The enzymatic reaction for compound testing was performed in a white 384-well polypropylene plate (Corning 3573) with a total reaction volume of 25 μl containing 9 nM Aurora B, 3 μM peptide substrate FAM-P21 (GL Biochem 116370), and 15 μM ATP (Sigma A7699-1G). The assay started with loading Aurora B diluted in kinase reaction buffer to wells, followed by addition of equal volume of 5× compounds for 15-min incubation at the room temperature for pre-treatment. The enzymatic reaction was initiated by addition of mixture of the substrate and ATP prepared in kinase reaction buffer. After incubation at 28° C. for one hour, 25 μl of stopper buffer (a mixture of 100 mM HEPES pH 7.5 buffer, 0.015% Brij-35, 50 mM EDTA and 0.2% of coating reagent 3 (Cliper Lifesciences)). After 30 minutes of incubation at room temperature, the plate was read in a Caliper. Percent of control was calculated as the percentage of compound-treated vs 2% DMSO vehicle-treated. The dose-response curves were generated and the IC₅₀ values were calculated by nonlinear sigmoid curve fitting using XLFit.

The IC₅₀ values (nM) of Aurora B biochemical activity for the examples disclosed herein are listed in Table 2.

### Cellular Assay

### TT Cell Proliferation Assay

Compounds disclosed herein were tested for the inhibition of RET by a cancer cell proliferation assay commonly known as MTT assay. In this assay, a complete media was prepared by adding 10% fetal bovine serum to RPMI-1640 medium (Life technology). TT cells were added to each of 88 wells of a 96 well plate at a seeding density of 6,000 cells/well/90 μL. The cells were allowed to attach to the plate by incubating at 37° C. for 24 hours. The compound was dissolved in DMSO (SIGMA). A solution of test compound was prepared in complete media by serial dilution to obtain the following concentrations: 50 μM, 15 μM, 5 μM, 1.5 μM, 0.5 μM, 0.15 μM, 0.05 μM, 0.015 μM and 0.005 μM. The test compound solution (10 μL) was added to each of 80 cell-containing wells. The final concentrations of the compound were following: 5 μM, 1.5 μM, 0.5 μM, 0.15 μM, 0.05 μM, 0.015 μM, 0.005 μM, 0.0015 μM and 0.0005 μM. The final concentration of DMSO is 0.5%. To the 8 remaining cell-containing wells, only complete media (containing 0.5% DMSO) was added to form a control group in order to measure maximal proliferation. To the remaining 8 empty wells, complete media was added to for a vehicle control group in order to measure background. The plates were incubated at 37° C. for 8 days. 10 μL WST-8 solution (DOJINDO, Cell Counting KIT-8) was added to each well. The plates were further incubated at 37° C. for 5 hours, and then read for the absorbance using a microplate reader at 450 nm. The IC₅₀ was calculated using GraphPad Prism.

### BAF3-KIF5B-RET, Ba/F3-KIF5B-RET-G810R, Ba/F3-TEL-RET-M918T and Ba/F3-KIF5B-RET-V804M Cell Proliferation Assays

Compounds disclosed herein were tested for the inhibition of RET by a cancer cell proliferation assay commonly known as CellTiter-Glo assay. In this assay, a complete media was prepared by adding 10% fetal bovine serum to RPMI-1640 medium (Life technology) for RET-driven cancer cells (BAF3-FIF5B-RET, Ba/F3-KIF5B-RET-G810R, Ba/F3-TEL-RET-M918T or Ba/F3-KIF5B-RET-V804M). Individual RET-driven type of cells were added to each of 88 wells of a 96 well plate at a seeding density of 2,000 cells/well/95 μL. The cells were allowed to attach to the plate by incubating at 37° C. for 24 hours. The compound was dissolved in DMSO (SIGMA). A solution of test compound was prepared in complete media by serial dilution to obtain the following concentrations: 20 μM, 6.67 μM, 2.22 μM, 0.74 μM, 0.25 μM, 0.082 μM, 0.027 μM, 0.0091 μM and 0.0030 μM. The test compound solution (5 μL) was added to each of 80 cell-containing wells. The final concentrations of the compound were following: 1 μM, 0.33 μM, 0.11 μM, 0.037 μM, 0.012 μM, 0.0041 μM, 0.0014 μM, 0.00046 μM and 0.00015 μM. The final concentration of DMSO is 0.1%. To the 8 remaining cell-containing wells, only complete media (containing 0.1% DMSO) was added to form a control group in order to measure maximal proliferation. To the remaining 8 empty wells, complete media was added to for a vehicle control group in order to measure background. The plates were incubated at 37° C. for 72 hours. 50 μl of CellTiter-Glo® Reagent was added to each well. Mix contents for 2 minutes on an orbital shaker to induce cell lysis. Incubate at room temperature for 10 minutes to stabilize luminescent signal. Record luminescence on Paradigm. Cell viability (CV %) was calculated relative to vehicle (DMSO) treated control wells. The IC₅₀ was calculated using GraphPad Prism.

The IC₅₀ values (nM) of growth inhibition in RET-driven cells for compounds disclosed are listed in Table 2 and Table 3.

### BAF3 Cell Proliferation Assay

Compounds disclosed herein were tested for the inhibition BAF3 cells proliferation without RET driven by CellTiter-Glo assay. In this assay, a complete media was prepared by adding 10% fetal bovine serum and 1 ng/ml IL-3 to RPMI-1640 medium (Life technology) for BAF3 cells. BAF3 cells were added to each of 88 wells of a 96 well plate at a seeding density of 2,000 cells/well/95 μL. The cells were allowed to attach to the plate by incubating at 37° C. for 24 hours. The compound was dissolved in DMSO (SIGMA). A solution of test compound was prepared in complete media by serial dilution to obtain the following concentrations: 200 μM, 66.7 μM, 22.2 μM, 7.4 μM, 2.5 μM, 0.82 μM, 0.27 μM, 0.091 μM and 0.0030 μM. The test compound solution (5 μL) was added to each of 80 cell-containing wells. The final concentrations of the compound were following: 10 μM, 3.33 μM, 1.11 μM, 0.37 μM, 0.12 μM, 0.041 μM, 0.014 μM, 0.0046 μM and 0.0015 μM. The final concentration of DMSO is 0.1%. To the 8 remaining cell-containing wells, only complete media (containing 0.1% DMSO) was added to form a control group in order to measure maximal proliferation. To the remaining 8 empty wells, complete media was added to for a vehicle control group in order to measure background. The plates were incubated at 37° C. for 72 hours. 50 μl of CellTiter-Glo® Reagent was added to each well. The contents were mixed for 2 minutes on an orbital shaker to induce cell lysis, and then were incubated at room temperature for 10 minutes to stabilize luminescent signal. Luminescence was recorded on Paradigm. Cell viability (CV %) was calculated relative to vehicle (DMSO) treated control wells. The IC₅₀ was calculated using GraphPad Prism.

The IC₅₀ values (nM) of growth inhibition in BAF3 cells for compounds disclosed are listed in Table 2 and Table 3.

TABLE 2

| | | | | | BA/F3- |
|---|---|---|---|---|---|
| | | | | | KIF5B- |
| | RET | KDR | AurB | BAF3 | RET |
| | Enzymatic | Enzymatic | Enzymatic | Cellular | Cellular |
| Example # | Activity | Activity | Activity | Activity | activity |
| 1 | | | | 1,015 | 1.8 |
| 2 | | | | 362 | 8.9 |
| 3 | | | | >10,000 | 110 |
| 4 | 0.67 | | 128 | 2,222 | 0.7 |
| 5 | 0.8 | | 19.5 | 516 | 3.0 |
| 6 | | | | 38.6 | 3.5 |
| 7 | | | | >10,000 | 48.1 |
| 8 | 0.38 | | 45 | 1,050 | 1.4 |
| 9 | 0.58 | | 80 | 1,132 | 0.6 |
| 10 | 3.5 | | 743 | >10,000 | 30.5 |
| 11 | 0.6 | | 66.4 | 3,063 | 0.9 |
| 12 | 0.4 | 2.0 | 25.2 | 1,049 | 1.0 |
| 13 | 0.4 | | 183 | 2,666 | 2.2 |
| 14 | | | | 1,003 | 2.8 |
| 15 | | | | 2,598 | 3.9 |
| 16 | | | | 1,077 | 2.2 |
| 17 | | | | 2,774 | 4.2 |
| 18 | 1.4 | 21 | | 1,172 | 0.7 |
| 19 | | | | 1,255 | 1.7 |
| 20 | | | | 1,480 | 6.7 |
| 21 | | | | 351 | 28.8 |
| 22 | | | | 756 | 0.7 |
| 23 | 1.7 | 4.2 | | 1,137 | 0.5 |
| 24 | | | | 2,540 | 3.5 |
| 25 | | | | 6,654 | 2.7 |
| 26 | | | | >10,000 | 46.8 |
| 27 | | | | 913 | 0.7 |
| 28 | | | | 2,326 | 4.0 |
| 32 | | | | 1,624 | 1.4 |
| 33 | | | | 2,579 | 5.1 |
| 34 | | | | 1,795 | 0.7 |
| 35 | | | | 340 | 0.5 |
| 36 | | | | 8,210 | 16.6 |
| 37 | 0.7 | | 138 | 4,460 | 1.2 |

TABLE 3

| | BA/F3- | BZ/F3-KIF5B- | BA/F3-KIF5B- | BA/F3-KIF5B- |
|---|---|---|---|---|
| Example | KIF5B- | RET | RET | RET |
| # | RET | V804M | G810R | M918T |
| 9 | 0.6 | 4.8 | 54.6 | 13.2 |
| 18 | 0.7 | 3.9 | 59.4 | 18.4 |
| Loxo-292 | 3.3 | 43.6 | 624 | 46.9 |

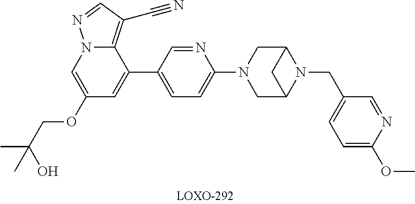 [SMILES: COc1ccc(CN2C3CC2CN(c2ccc(-c4cc(OCC(C)(C)O)cn5ncc(C#N)c45)cn2)C3)cn1]

What is claimed is:

1. A compound of Formula I: 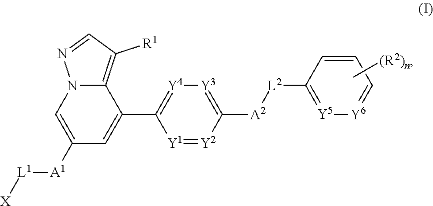
and/or a stereoisomer, stable isotope, or pharmaceutically acceptable salt or solvate thereof, wherein:
A¹ is a cyclic group selected from phenyl and 5- to 6-membered heteroaryl, wherein the 5- to 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl;
A² is a group selected from: 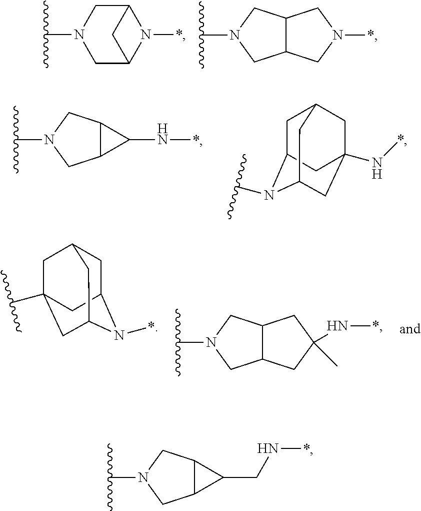
wherein the bond marked with an asterisk (*) represents the bond to L² of Formula I;
L¹ is a group selected from: 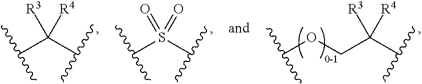
wherein R³ and R⁴ are each independently selected from H and C1-C6 alkyl, wherein the C1-C6 alkyl is optionally substituted with 1-3 groups independently selected from CN, halo, OH, and C1-C6 alkoxy; or wherein R³ and R⁴ are taken together to form a group selected from oxo, 3- to 6-membered cycloalkyl, and 5- to 6-membered heterocycles containing 1-2 heteroatoms independently selected from N, O, and S as ring members;
L² is a group selected from CO, SO₁₋₂, C1-C6 alkylenyl, and C1-C6 haloalkylenyl:
R¹ is selected from —CN, ethynyl, halo, —CF₃, —CH₃, —CH₂CH₃, cyclopropyl, —CH₂CN, and —CH(CN)CH₃;
each R² is independently selected from halo, —OR⁵, —N(R⁵)₂, —CN, C1-C6 alkyl, C1-C6 haloalkyl, C3-C6 cycloalkyl, —OC(O)R⁵, —CO₂R⁵, —C(O)N(R⁵)₂, —C(═NR⁶) N(R⁵)₂, —C(O)R⁵, —S(O)₀₋₂R⁷, —S(O)(═NR⁶)R⁷, —S(O)₁₋₂N(R⁵)₂, —N(R⁵)C(O)R⁷, —N(R⁵)C(═NR⁶) R⁷, —N(R⁵)S(O)₁₋₂R⁷, —N(R⁵)C(O)N(R⁵)₂, —N(R⁵)C(═NR⁶)N(R⁵)₂, —N(R⁵)S(O)₁₋₂N(R⁵)₂, and —N(R⁵)CO₂R⁷, wherein:
each R⁵ is independently selected from H, C1-C6 alkyl, C3-C6 cycloalkyl, C1-C6 haloalkyl, aryl, saturated and unsaturated 4-7 membered heterocyclyl containing 1-2 heteroatoms selected from N, O, and S as ring members, and heteroaryl containing 1-4 heteroatoms selected from N, O, and S as ring members;
R⁶ is independently selected from H, —CN, —OH, C1-C4 alkyl, and C1-C4 alkoxy;
R⁷ is independently selected from C1-C6 alkyl, C3-C6 cycloalkyl, C1-C6 haloalkyl, saturated and unsaturated 4-7 membered heterocyclyl containing 1-2 heteroatoms selected from N, O, and S as ring members, aryl, and heteroaryl containing 1-4 heteroatoms selected from N, O, and S as ring members;
X is selected from —OH, —NH₂, —CN, —NH(CO)(C1-C4 alkyl), C1-C4 alkyl, C1-C4 haloalkyl, and C1-C4 alkoxy;
Y¹, Y², Y³, Y⁴, Y⁵, and Y⁶ are independently selected from N and —CR⁸, wherein R⁸ is selected from H, F, Cl, CN, CH₃, and CF₃; and
n is an integer selected from 1-3.

2. The compound of claim 1, and/or a stereoisomer, a stable isotope, or a pharmaceutically acceptable salt or solvate thereof, wherein A¹ is a cyclic group selected from phenyl, pyridine, pyrimidine, pyrazine, pyridazine, pyrazole, thiophene, thiazole, and oxazole, and wherein the cyclic group is optionally substituted with 1-3 groups independently selected from halogen, CN, C1-C4 alkyl, and C1-C4 haloalkyl.

3. The compound of claim 1 or 2, and/or a stereoisomer, a stable isotope, or a pharmaceutically acceptable salt or solvate thereof, wherein A¹ is a cyclic group selected from: 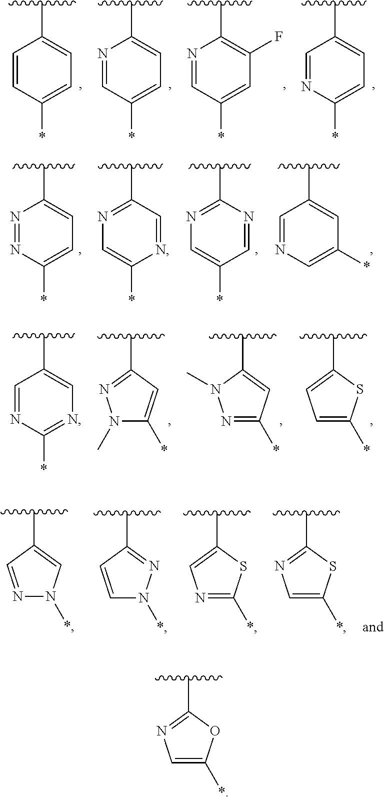
wherein the bond marked with an asterisk (*) represents the bond to L¹ of Formula I.

4. The compound of claim 1, and/or a stereoisomer, a stable isotope, or a pharmaceutically acceptable salt or solvate thereof, wherein A¹ is a cyclic group selected from 5- to 6-membered heteroaryl, wherein the 5- to 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl.

5. The compound of claim 1 or 4, and/or a stereoisomer, a stable isotope, or a pharmaceutically acceptable salt or solvate thereof, wherein A¹ is a cyclic group selected from pyridine, pyrimidine, pyrazine, pyridazine, pyrazole, thiophene, thiazole, and oxazole, and wherein the cyclic group is optionally substituted with 1-3 groups independently selected from halogen, CN, C1-C4 alkyl, and C1-C4 haloalkyl.

6. The compound of any one of claims 1-5, and/or a stereoisomer, a stable isotope, or a pharmaceutically acceptable salt or solvate thereof, wherein A¹ is a cyclic group selected from: 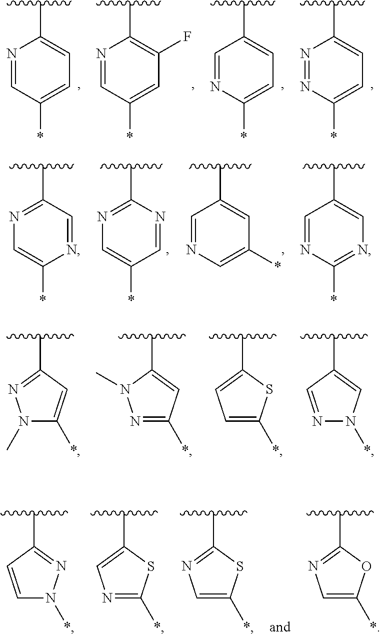
wherein the bond marked with an asterisk (*) represents the bond to L¹ of Formula I.

7. The compound of claim 1, and/or a stereoisomer, a stable isotope, or a pharmaceutically acceptable salt or solvate thereof, wherein A¹ is a cyclic group selected from 6-membered heteroaryl, wherein the 6-membered heteroaryl contains 1-2 heteroatoms independently selected from N, O, and S as ring members, and wherein the cyclic group is optionally substituted with 1-3 groups independently selected from halogen, CN, C1-C4 alkyl, and C1-C4 haloalkyl.

8. The compound of claim 1 or 7, and/or a stereoisomer, a stable isotope, or a pharmaceutically acceptable salt or solvate thereof, wherein A¹ is a cyclic group selected from pyridine, pyrimidine, pyrazine, and pyridazine, and wherein the cyclic group is optionally substituted with 1-3 groups independently selected from halogen, CN, C1-C4 alkyl, and C1-C4 haloalkyl.

9. The compound of any one of claims 1-8, and/or a stereoisomer, a stable isotope, or a pharmaceutically acceptable salt or solvate thereof, wherein A¹ is a cyclic group selected from: 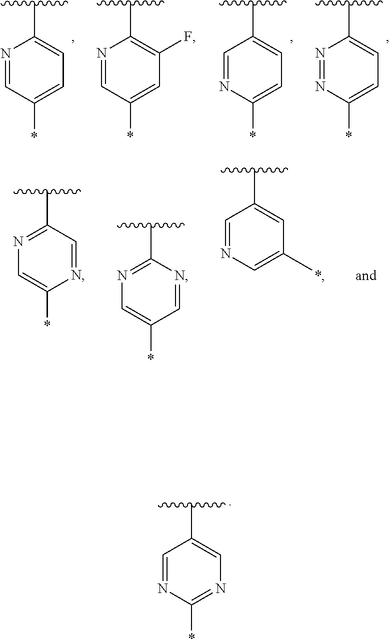
wherein the bond marked with an asterisk (*) represents the bond to L¹ of Formula I.

10. The compound of any one of claims 1-9, and/or a stereoisomer, a stable isotope, or a pharmaceutically acceptable salt or solvate thereof, wherein A² is a group selected from: 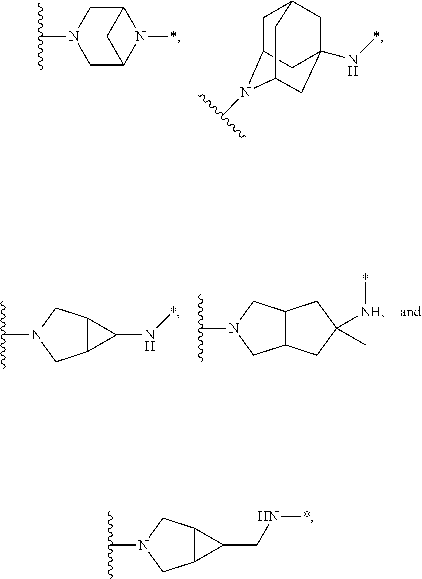
wherein the bond marked with an asterisk (*) represents the bond to L² of Formula I.

11. The compound of any one of claims 1-10, and/or a stereoisomer, a stable isotope, or a pharmaceutically acceptable salt or solvate thereof, wherein A² is 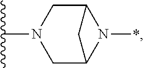
wherein the bond marked with an asterisk (*) represents the bond to L² of Formula I.

12. The compound of any one of claims 1-11, and/or a stereoisomer, a stable isotope, or a pharmaceutically acceptable salt or solvate thereof, wherein L¹ is a group selected from: 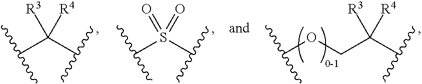
wherein R³ and R⁴ are independently selected from H and CH₃; or wherein R³ and R⁴ are taken together to form a group selected from oxo and 3- to 4-membered cycloalkyl.

13. The compound of any one of claims 1-12, and/or a stereoisomer, a stable isotope, or a pharmaceutically acceptable salt or solvate thereof, wherein L¹ is a group selected from: 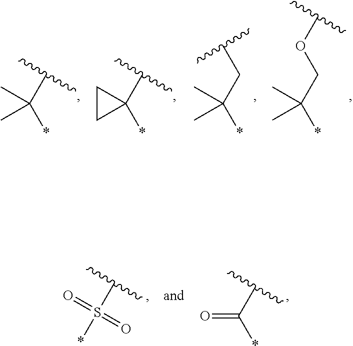
wherein the bond marked with an asterisk (*) represents the bond to X of Formula I.

14. The compound of any one of claims 1-13, and/or a stereoisomer, a stable isotope, or a pharmaceutically acceptable salt or solvate thereof, wherein L¹ is 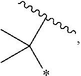 [SMILES: *C(*)(C)C]
wherein the bond marked with an asterisk (*) represents the bond to X of Formula I.

15. The compound of any one of claims 1-14, and/or a stereoisomer, a stable isotope, or a pharmaceutically acceptable salt or solvate thereof, wherein L² is a group selected from CO and C1-C6 alkylenyl.

16. The compound of any one of claims 1-15, and/or a stereoisomer, a stable isotope, or a pharmaceutically acceptable salt or solvate thereof, wherein L² is a group selected from CO and —CH₂—.

17. The compound of any one of claims 1-16, and/or a stereoisomer, a stable isotope, or a pharmaceutically acceptable salt or solvate thereof, wherein L² is —CH₂—.

18. The compound of any one of claims 1-17, and/or a stereoisomer, a stable isotope, or a pharmaceutically acceptable salt or solvate thereof, wherein R¹ is —CN.

19. The compound of any one of claims 1-18, and/or a stereoisomer, a stable isotope, or a pharmaceutically acceptable salt or solvate thereof, wherein each R² is independently selected from halogen, —OH, —CN, C1-C6 alkyl, C1-C6 haloalkyl, and C1-C6 alkoxy.

20. The compound of any one of claims 1-19, and/or a stereoisomer, a stable isotope, or a pharmaceutically acceptable salt or solvate thereof, wherein each R² is independently selected from halogen and C1-C6 alkoxy.

21. The compound of any one of claims 1-20, and/or a stereoisomer, a stable isotope, or a pharmaceutically acceptable salt or solvate thereof, wherein each R² is —OCH₃.

22. The compound of any one of claims 1-21, and/or a stereoisomer, a stable isotope, or a pharmaceutically acceptable salt or solvate thereof, wherein X is selected from —OH, —NH₂, —NH(CO)CH₃, and —CH₂CH₃.

23. The compound of any one of claims 1-22, and/or a stereoisomer, a stable isotope, or a pharmaceutically acceptable salt or solvate thereof, wherein X is selected from —OH and —NH₂.

24. The compound of any one of claims 1-23, and/or a stereoisomer, a stable isotope, or a pharmaceutically acceptable salt or solvate thereof, wherein X is —OH.

25. The compound of any one of claims 1-24, and/or a stereoisomer, a stable isotope, or a pharmaceutically acceptable salt or solvate thereof, wherein Y² is N, and Y¹, Y³, and Y⁴ are independently selected from —CR⁸, wherein R⁸ is selected from H, F, Cl, CN, CH₃, and CF₃.

26. The compound of any one of claims 1-24, and/or a stereoisomer, a stable isotope, or a pharmaceutically acceptable salt or solvate thereof, wherein Y² and Y⁴ are N, and Y¹ and Y³ are independently selected from —CR⁸, wherein R⁸ is selected from H, F, Cl, CN, CH₃, and CF₃.

27. The compound of any one of claims 1-24, and/or a stereoisomer, a stable isotope, or a pharmaceutically acceptable salt or solvate thereof, wherein Y¹ and Y³ are N, and Y² and Y⁴ are independently selected from —CR⁸, wherein R⁸ is selected from H, F, Cl, CN, CH₃, and CF₃.

28. The compound of any one of claims 1-27, and/or a stereoisomer, a stable isotope, or a pharmaceutically acceptable salt or solvate thereof, wherein Y⁵ is N and Y⁶ is selected from —CR⁸, wherein R⁸ is selected from H, F, Cl, CN, CH₃, and CF₃.

29. The compound of any one of claims 1-27, and/or a stereoisomer, a stable isotope, or a pharmaceutically acceptable salt or solvate thereof, wherein Y⁶ is N and Y⁵ is selected from —CR⁸, wherein R⁸ is selected from H, F, Cl, CN, CH₃, and CF₃.

30. The compound of any one of claims 1-29, and/or a stereoisomer, a stable isotope, or a pharmaceutically acceptable salt or solvate thereof, wherein R⁸ is H.

31. The compound of any one of claims 1-30, and/or a stereoisomer, a stable isotope, or a pharmaceutically acceptable salt or solvate thereof, wherein n is 1.

32. The compound of claim 1, wherein the compound is selected from compounds of Formula IA: 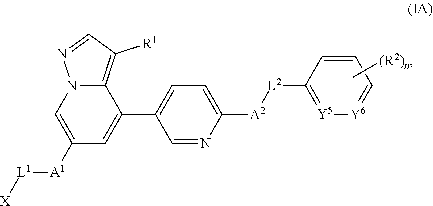
and/or a stereoisomer, a stable isotope, or a pharmaceutically acceptable salt or solvate thereof, wherein A¹, A², L¹, L², R¹, R², X, Y⁵, Y⁶, and n are the same as those defined in claim 1.

33. The compound of claim 1, wherein the compound is selected from compounds of Formula IB: 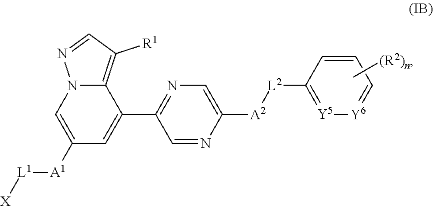
and/or a stereoisomer, a stable isotope, or a pharmaceutically acceptable salt or solvate thereof, wherein A¹, A², L¹, L², R¹, R², X, Y⁵, Y⁶, and n are the same as those defined in claim 1.

34. The compound of claim 1, wherein the compound is selected from compounds of Formula IC: 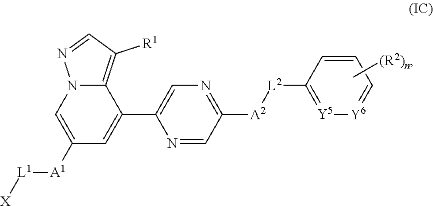
and/or a stereoisomer, a stable isotope, or a pharmaceutically acceptable salt or solvate thereof, wherein A¹, A², L¹, L², R¹, R², X, Y⁵, Y⁶, and n are the same as those defined in claim 1.

35. The compound of claim 1, wherein the compound is selected from compounds of Formula ID: 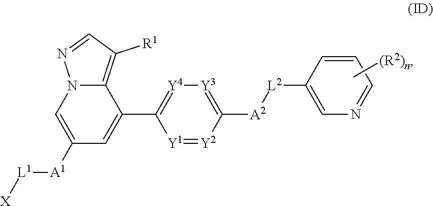
and/or a stereoisomer, a stable isotope, or a pharmaceutically acceptable salt or solvate thereof, wherein A¹, A², L¹, L², R¹, R², X, Y¹, Y², Y³, Y⁴, and n are the same as those defined in claim 1.

36. The compound of claim 1, wherein the compound is selected from compounds of Formula IE: 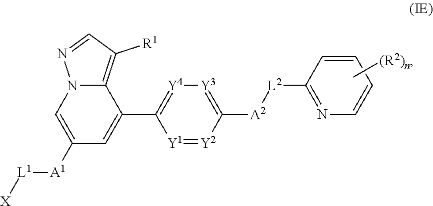
and/or a stereoisomer, a stable isotope, or a pharmaceutically acceptable salt or solvate thereof, wherein A¹, A², L¹, L², R¹, R², X, Y¹, Y², Y³, Y⁴, and n are the same as those defined in claim 1.

37. The compound of claim 1, wherein the compound is selected from compounds of Formula IIA: 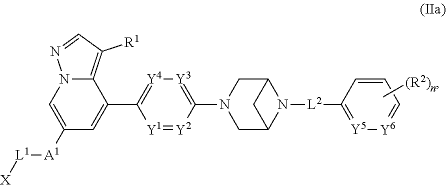
and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in claim 1.

38. The compound of claim 1, wherein the compound is selected from compounds of Formula IIB: 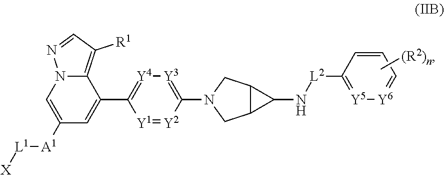
and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in claim 1.

39. The compound of claim 1, wherein the compound is selected from compounds of Formula IIC: 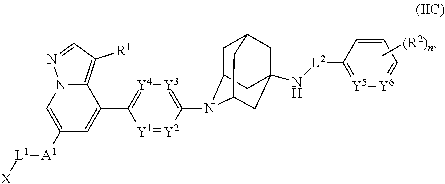
and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in claim 1.

40. The compound of claim 1, wherein the compound is selected from compounds of Formula IID: 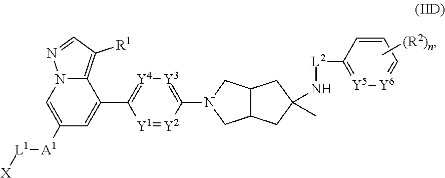
and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in claim 1.

41. The compound of claim 1, wherein the compound is selected from compounds of Formula IIE: 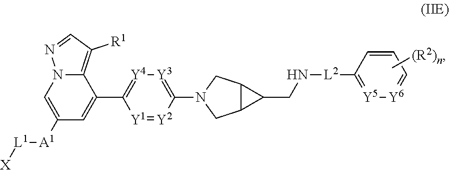
and/or a stereoisomer, a stable isotope, or a pharmaceutically acceptable salt or solvate thereof, wherein A¹, L¹, L², R¹, R², X, Y¹, Y², Y³, Y⁴, Y⁵, Y⁶, and n are the same as those defined in claim 1.

42. A compound selected from the following compounds, and/or a stereoisomer, a stable isotope, or a pharmaceutically acceptable salt or solvate thereof:

| Example # | Structure | Chemical Name |
|---|---|---|
| 1 | 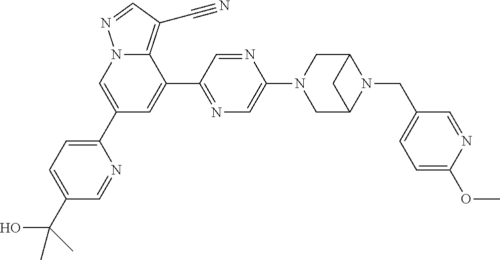 [SMILES: COc1ccc(CN2C3CC2CN(c2cnc(-c4cc(-c5ccc(C(C)(C)O)cn5)cn5ncc(C#N)c45)cn2)C3)cn1] | 6-(5-(2-hydroxypropan-2-yl)pyridin-2- yl)-4-(5-(6-((6-methoxypyridin-3- yl)methyl)-3,6-diazabicyclo[3.1.1]heptan- 3-yl)pyrazin-2-yl)pyrazolo[1,5- a]pyridine-3-carbonitrile, |
| 2 | 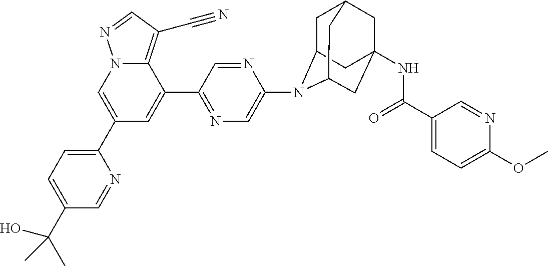 [SMILES: COc1ccc(C(=O)NC23CC4CC(C2)N(c2cnc(-c5cc(-c6ccc(C(C)(C)O)cn6)cn6ncc(C#N)c56)cn2)C(C4)C3)cn1] | N-((1R,3S,5s,7s)-2-(5-(3-cyano-6-(5-(2- hydroxypropan-2-yl)pyridin-2- yl)pyrazolo[1,5-a]pyridin-4-yl)pyrazin-2- yl)-2-azaadamantan-5-yl)-6- methoxynicotinamide, |
| 3 | 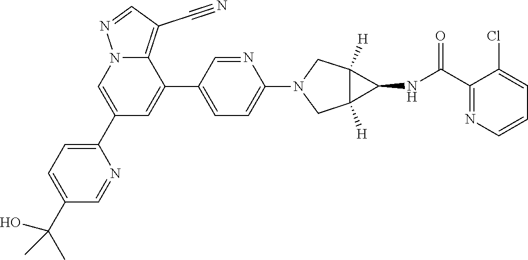 [SMILES: CC(C)(O)c1ccc(-c2cc(-c3ccc(N4C[C@@H]5[C@H](C4)[C@@H]5NC(=O)c4ncccc4Cl)nc3)c3c(C#N)cnn3c2)nc1] | 3-chloro-N-((1R,5S,6r)-3-(5-(3-cyano-6- (5-(2-hydroxypropan-2-yl)pyridin-2- yl)pyrazolo[1,5-a]pyridin-4-yl)pyridin-2- yl)-3-azabicyclo[3.1.0]hexan-6- yl)picolinamide, |
| 4 | 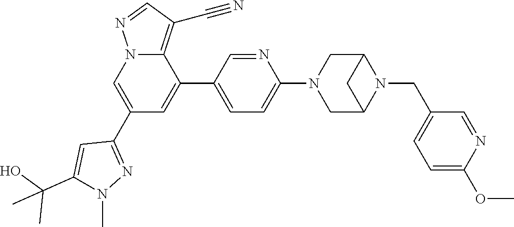 [SMILES: COc1ccc(CN2C3CC2CN(c2ccc(-c4cc(-c5cc(C(C)(C)O)n(C)n5)cn5ncc(C#N)c45)cn2)C3)cn1] | 6-(5-(2-hydroxypropan-2-yl)-1-methyl- 1H-pyrazol-3-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 5 | 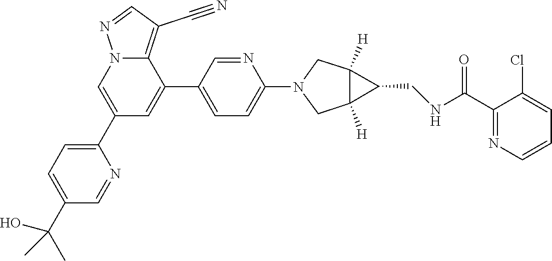 [SMILES: CC(C)(O)c1ccc(-c2cc(-c3ccc(N4C[C@@H]5[C@@H](CNC(=O)c6ncccc6Cl)[C@@H]5C4)nc3)c3c(C#N)cnn3c2)nc1] | 3-chloro-N-(((1R,5S,6s)-3-(5-(3-cyano-6- (5-(2-hydroxypropan-2-yl)pyridin-2- yl)pyrazolo[1,5-a]pyridin-4-yl)pyridin-2- yl)-3-azabicyclo[3.1.0]hexan-6- yl)methyl)picolinamide, |
| 6 | 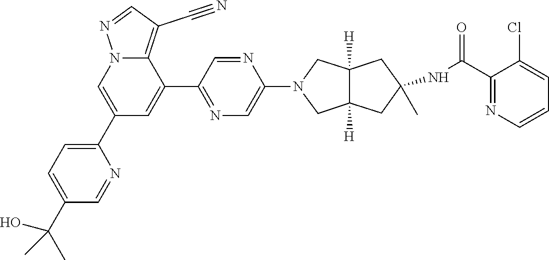 [SMILES: CC(C)(O)c1ccc(-c2cc(-c3cnc(N4C[C@@H]5C[C@@](C)(NC(=O)c6ncccc6Cl)C[C@@H]5C4)cn3)c3c(C#N)cnn3c2)nc1] | 3-chloro-N-((3aR,5s,6aS)-2-(5-(3-cyano- 6-(5-(2-hydroxypropan-2-yl)pyridin-2- yl)pyrazolo[1,5-a]pyridin-4-yl)pyrazin-2- yl)-5- methyloctahydrocyclopenta[c]pyrrol-5- yl)picolinamide, |
| 7 | 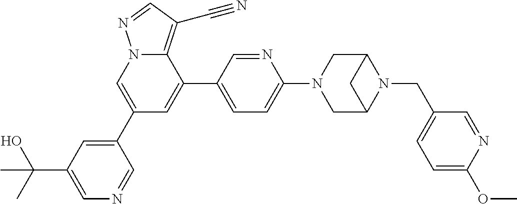 [SMILES: COc1ccc(CN2C3CC2CN(c2ccc(-c4cc(-c5cncc(C(C)(C)O)c5)cn5ncc(C#N)c45)cn2)C3)cn1] | 6-(5-(2-hydroxypropan-2-yl)pyridin-3- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 8 | 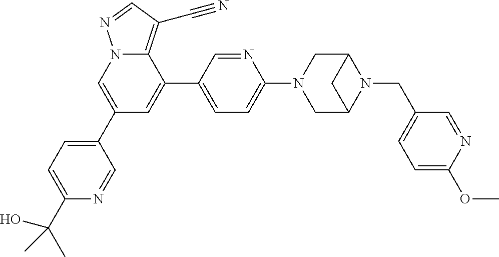 [SMILES: COc1ccc(CN2C3CC2CN(c2ccc(-c4cc(-c5ccc(C(C)(C)O)nc5)cn5ncc(C#N)c45)cn2)C3)cn1] | 6-(6-(2-hydroxypropan-2-yl)pyridin-3- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 9 | 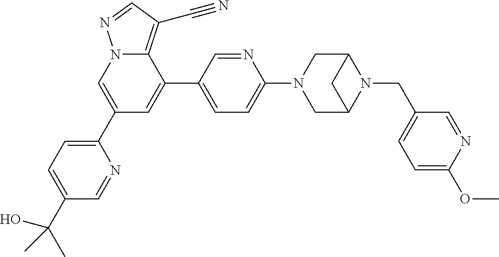 [SMILES: COc1ccc(CN2C3CC2CN(c2ccc(-c4cc(-c5ccc(C(C)(C)O)cn5)cn5ncc(C#N)c45)cn2)C3)cn1] | 6-(5-(2-hydroxypropan-2-yl)pyridin-2- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 10 | 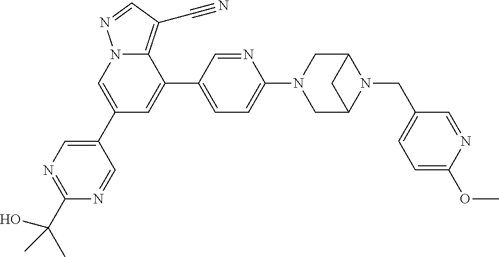 [SMILES: COc1ccc(CN2C3CC2CN(c2ccc(-c4cc(-c5cnc(C(C)(C)O)nc5)cn5ncc(C#N)c45)cn2)C3)cn1] | 6-(2-(2-hydroxypropan-2-yl)pyrimidin-5- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 11 | 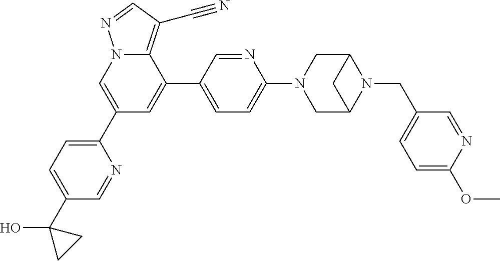 [SMILES: COc1ccc(CN2C3CC2CN(c2ccc(-c4cc(-c5ccc(C6(O)CC6)cn5)cn5ncc(C#N)c45)cn2)C3)cn1] | 6-(5-(1-hydroxycyclopropyl)pyridin-2- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 12 | 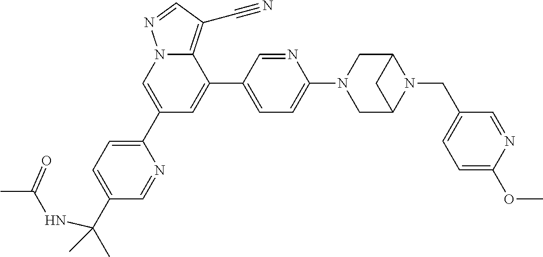 [SMILES: COc1ccc(CN2C3CC2CN(c2ccc(-c4cc(-c5ccc(C(C)(C)NC(C)=O)cn5)cn5ncc(C#N)c45)cn2)C3)cn1] | N-(2-(6-(3-cyano-4-(6-(6-((6- methoxypyridin-3-yl)methyl)-3,6- diazabicyclo[3.1.1]heptan-3-yl)pyridin-3- yl)pyrazolo[1,5-a]pyridin-6-yl)pyridin-3- yl)propan-2-yl)acetamide, |
| 13 | 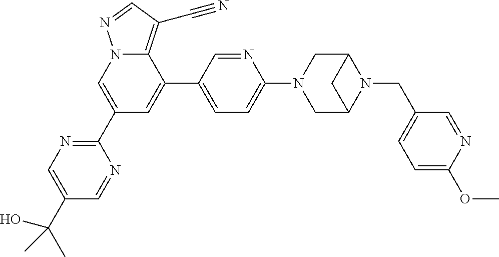 [SMILES: COc1ccc(CN2C3CC2CN(c2ccc(-c4cc(-c5ncc(C(C)(C)O)cn5)cn5ncc(C#N)c45)cn2)C3)cn1] | 6-(5-(2-hydroxypropan-2-yl)pyrimidin-2- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 14 | 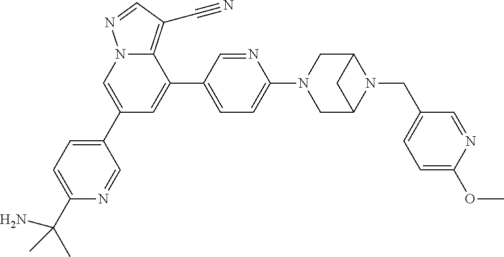 [SMILES: COc1ccc(CN2C3CC2CN(c2ccc(-c4cc(-c5ccc(C(C)(C)N)nc5)cn5ncc(C#N)c45)cn2)C3)cn1] | 6-(6-(2-aminopropan-2-yl)pyridin-3-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 15 | 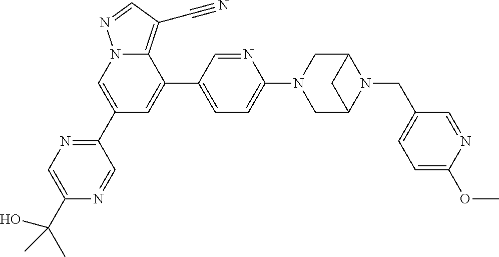 [SMILES: COc1ccc(CN2C3CC2CN(c2ccc(-c4cc(-c5cnc(C(C)(C)O)cn5)cn5ncc(C#N)c45)cn2)C3)cn1] | 6-(5-(2-hydroxypropan-2-yl)pyrazin-2- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 16 | 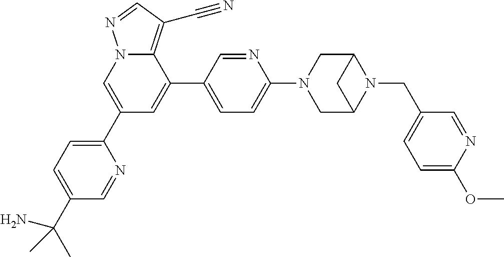 [SMILES: COc1ccc(CN2C3CC2CN(c2ccc(-c4cc(-c5ccc(C(C)(C)N)cn5)cn5ncc(C#N)c45)cn2)C3)cn1] | 6-(5-(2-aminopropan-2-yl)pyridin-2-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 17 | 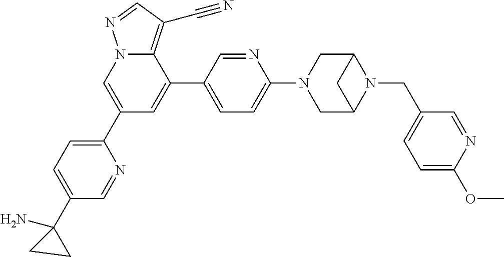 [SMILES: COc1ccc(CN2C3CC2CN(c2ccc(-c4cc(-c5ccc(C6(N)CC6)cn5)cn5ncc(C#N)c45)cn2)C3)cn1] | 6-(5-(1-aminocyclopropyl)pyridin-2-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 18 | 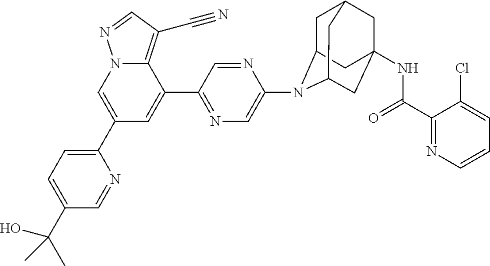 [SMILES: CC(C)(O)c1ccc(-c2cc(-c3cnc(N4C5CC6CC4CC(NC(=O)c4ncccc4Cl)(C6)C5)cn3)c3c(C#N)cnn3c2)nc1] | 3-chloro-N-((1R,3S,5s,7s)-2-(5-(3-cyano- 6-(5-(2-hydroxypropan-2-yl)pyridin-2- yl)pyrazolo[1,5-a]pyridin-4-yl)pyrazin-2- yl)-2-azaadamantan-5-yl)picolinamide, |
| 19 | 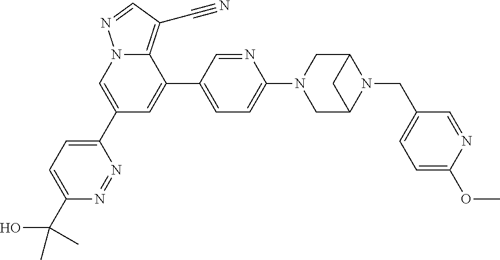 [SMILES: COc1ccc(CN2C3CC2CN(c2ccc(-c4cc(-c5ccc(C(C)(C)O)nn5)cn5ncc(C#N)c45)cn2)C3)cn1] | 6-(6-(2-hydroxypropan-2-yl)pyridazin-3- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 20 | 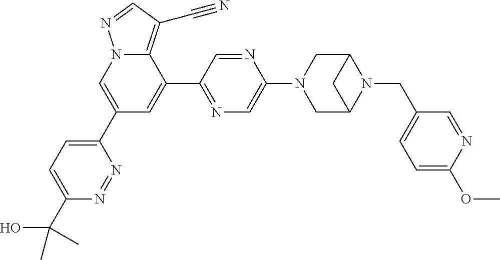 [SMILES: COc1ccc(CN2C3CC2CN(c2cnc(-c4cc(-c5ccc(C(C)(C)O)nn5)cn5ncc(C#N)c45)cn2)C3)cn1] | 6-(6-(2-hydroxypropan-2-yl)pyridazin-3- yl)-4-(5-(6-((6-methoxypyridin-3- yl)methyl)-3,6-diazabicyclo[3.1.1 ]heptan- 3-yl)pyrazin-2-yl)pyrazolo[1,5- a]pyridine-3-carbonitrile, |
| 21 | 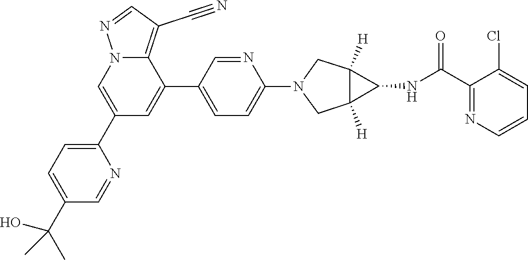 [SMILES: CC(C)(O)c1ccc(-c2cc(-c3ccc(N4C[C@@H]5[C@H](C4)[C@H]5NC(=O)c4ncccc4Cl)nc3)c3c(C#N)cnn3c2)nc1] | 3-chloro-N-((1R,5S,6s)-3-(5-(3-cyano-6- (5-(2-hydroxypropan-2-yl)pyridin-2- yl)pyrazolo[1,5-a]pyridin-4-yl)pyridin-2- yl)-3-azabicyclo[3.1.0]hexan-6- yl)picolinamide, |
| 22 | 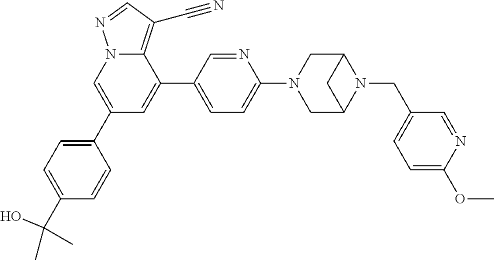 [SMILES: COc1ccc(CN2C3CC2CN(c2ccc(-c4cc(-c5ccc(C(C)(C)O)cc5)cn5ncc(C#N)c45)cn2)C3)cn1] | 6-(4-(2-hydroxypropan-2-yl)phenyl)-4-(6- (6-((6-methoxypyridin-3-yl)methyl)-3,6- diazabicyclo[3.1.1]heptan-3-yl)pyridin-3- yl)pyrazolo[1,5-a]pyridine-3-carbonitrile, |
| 23 | 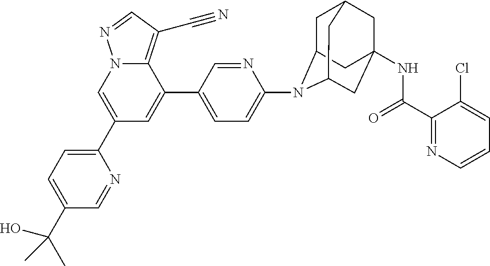 [SMILES: CC(C)(O)c1ccc(-c2cc(-c3ccc(N4C5CC6CC4CC(NC(=O)c4ncccc4Cl)(C6)C5)nc3)c3c(C#N)cnn3c2)nc1] | 3-chloro-N-((1R,3S,5s,7s)-2-(5-(3-cyano- 6-(5-(2-hydroxypropan-2-yl)pyridin-2- yl)pyrazolo[1,5-a]pyridin-4-yl)pyridin-2- yl)-2-azaadamantan-5-yl)picolinamide, |
| 24 | 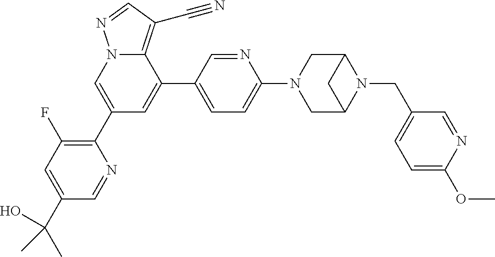 [SMILES: COc1ccc(CN2C3CC2CN(c2ccc(-c4cc(-c5ncc(C(C)(C)O)cc5F)cn5ncc(C#N)c45)cn2)C3)cn1] | 6-(3-fluoro-5-(2-hydroxypropan-2- yl)pyridin-2-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 25 | 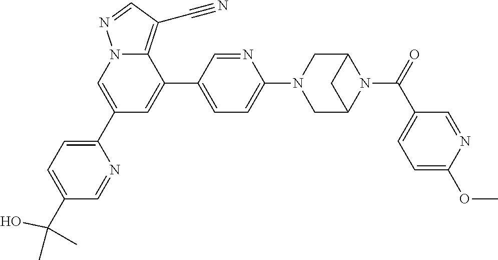 [SMILES: COc1ccc(C(=O)N2C3CC2CN(c2ccc(-c4cc(-c5ccc(C(C)(C)O)cn5)cn5ncc(C#N)c45)cn2)C3)cn1] | 6-(5-(2-hydroxypropan-2-yl)pyridin-2- yl)-4-(6-(6-(6-methoxynicotinoyl)-3,6- diazabicyclo[3.1.1]heptan-3-yl)pyridin-3- yl)pyrazolo[1,5-a]pyridine-3-carbonitrile, |
| 26 | 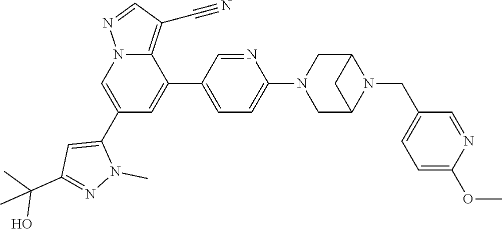 [SMILES: COc1ccc(CN2C3CC2CN(c2ccc(-c4cc(-c5cc(C(C)(C)O)nn5C)cn5ncc(C#N)c45)cn2)C3)cn1] | 6-(3-(2-hydroxypropan-2-yl)-1-methyl- 1H-pyrazol-5-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 27 | 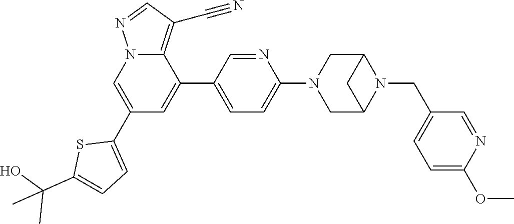 [SMILES: COc1ccc(CN2C3CC2CN(c2ccc(-c4cc(-c5ccc(C(C)(C)O)s5)cn5ncc(C#N)c45)cn2)C3)cn1] | 6-(5-(2-hydroxypropan-2-yl)thiophen-2- yl)-4-(6-(6-((6-methoxypyridin-3- yl)methyl)-3,6-diazabicyclo[3.1.1]heptan- 3-yl)pyridin-3-yl)pyrazolo[1,5-a]pyridine- 3-carbonitrile, |
| 28 | 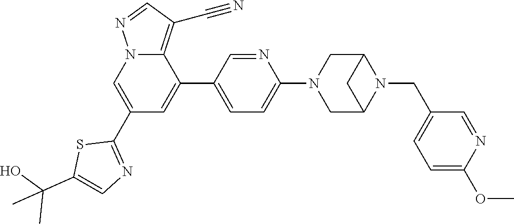 [SMILES: COc1ccc(CN2C3CC2CN(c2ccc(-c4cc(-c5ncc(C(C)(C)O)s5)cn5ncc(C#N)c45)cn2)C3)cn1] | N-(2-(3-(3-cyano-4-(6-(6-((6- methoxypyridin-3-yl)methyl)-3,6- diazabicyclo[3.1.1]heptan-3-yl)pyridin-3- yl)pyrazolo[1,5-a]pyridin-6-yl)-1-methyl- 1H-pyrazol-5-yl)propan-2-yl)acetamide, |
| 29 | 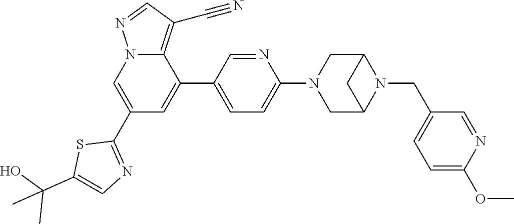 [SMILES: COc1ccc(CN2C3CC2CN(c2ccc(-c4cc(-c5ncc(C(C)(C)O)s5)cn5ncc(C#N)c45)cn2)C3)cn1] | 6-(5-(2-hydroxypropan-2-yl)thiazol-2-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 30 | 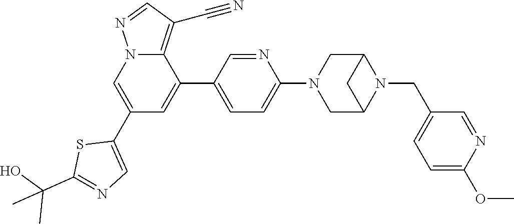 [SMILES: COc1ccc(CN2C3CC2CN(c2ccc(-c4cc(-c5cnc(C(C)(C)O)s5)cn5ncc(C#N)c45)cn2)C3)cn1] | 6-(2-(2-hydroxypropan-2-yl)thiazol-5-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 31 | 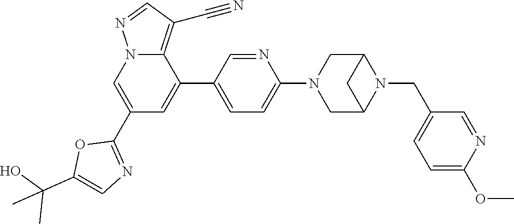 [SMILES: COc1ccc(CN2C3CC2CN(c2ccc(-c4cc(-c5ncc(C(C)(C)O)o5)cn5ncc(C#N)c45)cn2)C3)cn1] | 6-(5-(2-hydroxypropan-2-yl)oxazol-2-yl)- 4-(6-(6-((6-methoxypyridin-3-yl)methyl)- 3,6-diazabicyclo[3.1.1]heptan-3- yl)pyridin-3-yl)pyrazolo[1,5-a]pyridine-3- carbonitrile, |
| 32 | 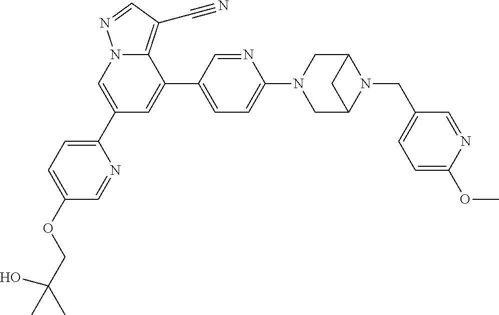 [SMILES: COc1ccc(CN2C3CC2CN(c2ccc(-c4cc(-c5ccc(OCC(C)(C)O)cn5)cn5ncc(C#N)c45)cn2)C3)cn1] | 6-(5-(2-hydroxy-2- methylpropoxy)pyridin-2-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 33 | 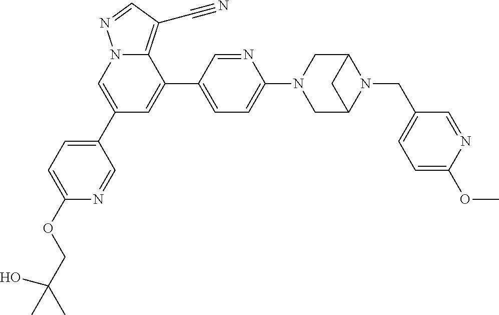 [SMILES: COc1ccc(CN2C3CC2CN(c2ccc(-c4cc(-c5ccc(OCC(C)(C)O)nc5)cn5ncc(C#N)c45)cn2)C3)cn1] | 6-(6-(2-hydroxy-2- methylpropoxy)pyridin-3-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 34 | 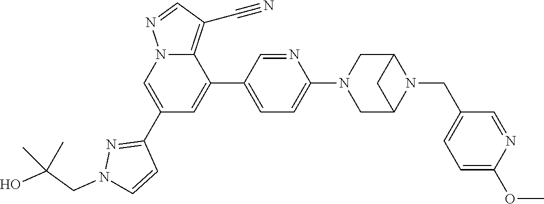 [SMILES: COc1ccc(CN2C3CC2CN(c2ccc(-c4cc(-c5ccn(CC(C)(C)O)n5)cn5ncc(C#N)c45)cn2)C3)cn1] | 6-(1-(2-hydroxy-2-methylpropyl)-1H- pyrazol-3-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 35 | 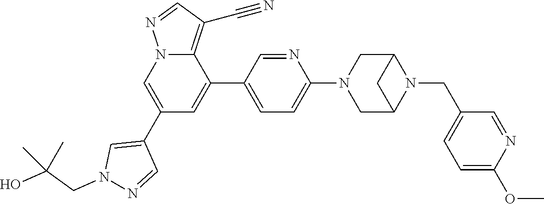 [SMILES: COc1ccc(CN2C3CC2CN(c2ccc(-c4cc(-c5cnn(CC(C)(C)O)c5)cn5ncc(C#N)c45)cn2)C3)cn1] | 6-(1-(2-hydroxy-2-methylpropyl)-1H- pyrazol-4-yl)-4-(6-(6-((6- methoxypyridin-3-yl)methyl)-3,6- diazabicyclo[3.1.1]heptan-3-yl)pyridin-3- yl)pyrazolo[1,5-a]pyridine-3-carbonitrile, |
| 36 | 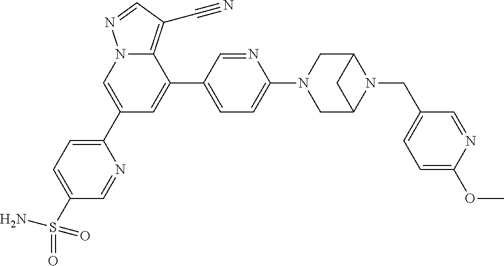 [SMILES: COc1ccc(CN2C3CC2CN(c2ccc(-c4cc(-c5ccc(S(N)(=O)=O)cn5)cn5ncc(C#N)c45)cn2)C3)cn1] | 6-(3-cyano-4-(6-(6-((6-methoxypyridin-3- yl)methyl)-3,6-diazabicyclo[3.1.1]heptan- 3-yl)pyridin-3-yl)pyrazolo[1,5-a]pyridin- 6-yl)pyridine-3-sulfonamide, and |
| 37 | 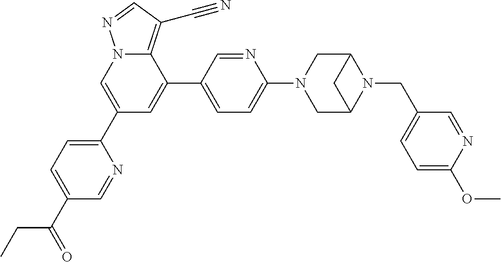 [SMILES: CCC(=O)c1ccc(-c2cc(-c3ccc(N4CC5CC(C4)N5Cc4ccc(OC)nc4)nc3)c3c(C#N)cnn3c2)nc1] | 4-(6-(6-((6-methoxypyridin-3-yl)methyl)- 3,6-diazabicyclo[3.1.1]heptan-3- yl)pyridin-3-yl)-6-(5-propionylpyridin-2- yl)pyrazolo[1,5-a]pyridine-3-carbonitrile. |

43. A pharmaceutical composition comprising a compound of any one of claims 1-42, and/or a stereoisomer, a stable isotope, or a pharmaceutically acceptable salt or solvate thereof, admixed with at least one pharmaceutically acceptable carrier.

44. The pharmaceutical composition of claim 43, further comprising at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, another RET kinase inhibitor, and kinase inhibitors.

45. The pharmaceutical composition of claim 44, wherein the at least one therapeutic co-agent or co-treatment is combined with the compound in a single dosage form, or the at least one therapeutic co-agent is administered simultaneously or sequentially as separate dosage forms.

46. A method to treat a RET-associated disease in a patient in need thereof, comprising administering to the patient in need of such treatment a therapeutically effective amount of a compound of any one of claims 1-42, and/or a stereoisomer, a stable isotope, or a pharmaceutically acceptable salt or solvate thereof, or a pharmaceutical composition of any one of claims 43-45.

47. The method of claim 46, wherein the method comprises determining if the disease in the patient is a RET-associated disease, and administering to the patient in need of such treatment a therapeutically effective amount of a compound of any one of claims 1-42, and/or a stereoisomer, a stable isotope, or a pharmaceutically acceptable salt or solvate thereof, or a pharmaceutical composition of any one of claims 43-45.

48. The method of claim 46 or claim 47, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

49. The method of claim 46 or claim 47, wherein the RET-associated disease is irritable bowel syndrome or other gastrointestinal disorder having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

50. The method of claim 48, wherein the treatment comprises administering at least one therapeutic co-agent or co-treatment selected from chemotherapeutics and other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

51. The method of claim 50, wherein the administering the compound is conducted simultaneously or serially with the administering of the at least one therapeutic co-agent.

52. The method of claim 51, wherein the administering of the at least one therapeutic co-agent comprises the administering of another RET inhibitor, an immunotherapy, or combination thereof.

53. The method of claim 48, wherein the RET-associated cancer is selected from lung cancer, papillary thyroid cancer, medullary thyroid cancer, differentiated thyroid cancer, recurrent thyroid cancer, refractory differentiated thyrpoid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, overian cancer, and myeloproliferative cancer.

54. The method of any of one of claims 46-53, wherein the compound of any one of claims 1-42, and/or a stereoisomer, a stable isotope, or a pharmaceutically acceptable salt or solvate thereof, or a pharmaceutical composition of any one of claims 43-45, is orally administered.

55. A use of a compound of any one of claims 1-42, and/or a stereoisomer, a stable isotope, or a pharmaceutically acceptable salt or solvate thereof, or a pharmaceutical composition according to any one of claims 42-45, as a medicament, in the manufacture of a medicament, or in medicine for treatment of a RET-associated disease.

56. The use of claim 55, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

57. The use of claim 56, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

58. The use of claim 56 or claim 57, wherein the RET-associated cancer is selected from lung cancer, papillary thyroid cancer, medullary thyroid cancer, differentiated thyroid cancer, recurrent thyroid cancer, refractory differentiated thyrpoid cancer, multiple endocrine neoplasia type 2A or 2B (MEN2A or MEN 2B, respectively), pheochromocytoma, parathyroid hyperplasia, breast cancer, pancreative cancer, salivary gland cancer, spitz tumors, colorectal cancer, papillary renal cell carcinoma, ganglioneuromatosis of the gastroenteric mucosa, cervical cancer, overian cancer, and myeloproliferative cancer.

59. The use of any of one of claims 55-58, wherein the medicament is formulated for oral administration.

60. A compound of any one of claims 1-42, and/or a stereoisomer, a stable isotope, or a pharmaceutically acceptable salt or solvate thereof, or a pharmaceutical composition of claims 43-45, for use in treating a RET-associated disease.

61. The compound or pharmaceutical composition for use of claim 60, wherein the RET-associated disease is a RET-associated cancer having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

62. The compound or pharmaceutical composition for use of claim 60, wherein the RET-associated disease is irritable bowel syndrome or other gastrointestinal disorders having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein.

63. The compound or pharmaceutical composition for use of claim 60 or claim 61, wherein the RET-associated disease is a RET-associated cancer, and the use comprises determining if the cancer in a patient is RET-associated cancer, and administering to the patient in need of such treatment a therapeutically effective amount of the compound or pharmaceutical composition.

64. The compound or pharmaceutical composition for use of claim 61 or claim 63, wherein the RET-associated cancer is selected from lung cancer, papillary thyroid cancer, medullary thyroid cancer, differentiated thyroid cancer, recurrent thyroid cancer, refractory differentiated thyrpoid cancer, multiple endocrine neoplasia type 2A or 2B (MEN2A or MEN 2B, respectively), pheochromocytoma, parathyroid hyperplasia, breast cancer, pancreatic cancer, salivary gland cancer, spitz tumors, colorectal cancer, papillary renal cell carcinoma, ganglioneuromatosis of the gastroenteric mucosa, cervical cancer, overian cancer, and myeloproliferative cancer.

65. A method of inhibiting RET kinase activity in vitro or in vivo for a RET-associated cancer cell having a RET gene fusion, one or more point mutations in RET gene, or a RET gene amplification that results in overexpression of a RET gene leading to a pathogenic increase in the activity of a kinase domain of a RET protein or a constitutively active kinase domain of a RET protein, with a compound of any one of claims 1-42, and/or a stereoisomer, a stable isotope, or a pharmaceutically acceptable salt or solvate thereof.

66. A method of treating RET-associated cancer in a patient who has developed resistance to a RET inhibitor, comprising administering to the patient in need of such treatment a therapeutically effective amount of a compound of any one of claims 1-42, and/or a stereoisomer, a stable isotope, or a pharmaceutically acceptable salt or solvate thereof, or a pharmaceutical composition of any one of claims 43-45.

67. The method of claim 66, wherein the method comprises (a) determining the RET-mutations of a cancer cell in a sample from the patient who developed resistance to a prior treatment of a RET inhibitor; and (b) administering a compound of any one of claims 1-42, and/or a stereoisomer, a stable isotope, or a pharmaceutically acceptable salt or solvate thereof, or a pharmaceutical composition of any one of claims 43-45.

68. The method of claim 66 or claim 67, wherein the prior treatment comprises a administering of at least one therapeutic co-agent or co-treatment selected from chemotherapeutics or other anti-cancer agents, apoptosis modulators, immune enhancers, agents for immunotherapy, immune checkpoint inhibitors, radiation, anti-tumor vaccines, agents for cytokine therapy, signal transduction inhibitors, and kinase inhibitors.

69. The method of claim 68, wherein the administering of the therapeutic co-agent comprises an administering of another RET inhibitor, an immunotherapy, or combination thereof.

70. A kit comprising a compound of any of claims 1-42 or a pharmaceutically acceptable salt thereof, or a pharmaceutical composition according to any of claims 43-45, and a therapeutic co-agent.
